(19) United States
(12) Patent Application Publication
(10) Pub. No.: US 2025/0025490 A1
(43) Pub. Date: Jan. 23, 2025

(54) INDUCTION OF TRAINED IMMUNITY FOR THE TREATMENT OF HYPERPROLIFERATIVE DISORDERS

(71) Applicant: UNIVERSITY OF LOUISVILLE RESEARCH FOUNDATION, INC., Louisville, KY (US)

(72) Inventor: Jun YAN, Louisville, KY (US)

(21) Appl. No.: 18/683,142

(22) PCT Filed: Aug. 12, 2022
(86) PCT No.: PCT/US2022/040169

§ 371 Date: Feb. 12, 2024

Related U.S. Application Data
(60) Provisional application No. 63/232,989, filed on Aug. 13, 2021.

(51) Int. Cl. A61K 31/716; A61K 9/00; A61K 36/064; A61P 35/04; C07K 16/00

(52) U.S. Cl. CPC A61K 31/716; A61K 9/0053; A61K 36/064; A61P 35/04; C07K 16/00

(57) ABSTRACT

In certain embodiments, the present invention provides a method of treating a pancreatic disorder comprising administering a β-glucan. In certain embodiments, the present invention provides a method inducing influx of immune cells to a pancreas comprising administering a yeast-derived particulate β-glucan. In certain embodiments, the present invention provides a method of reducing tumor in a pancreas comprising administering a β-glucan. In certain embodiments, the present invention provides a method of recruiting anti-tumor, innate immune cells comprising administering a β-glucan. In certain embodiments, the present invention provides a method of inducing whole β-Glucan particles trained immunity in a cancer. In certain embodiments, the present invention provides a method of inhibiting cancer metastasis comprising administering WGP. In certain embodiments, the present invention provides a method of developing trained innate immune cells as an adoptive cell therapy in cancer. In certain embodiments, the present invention provides isolated or purified beta-glucan-trained innate immune cells.

### CROSS-REFERENCE TO RELATED APPLICATION

This application claims priority to U.S. Provisional Application No. 63/232,989 that was filed on Aug. 13, 2021. The entire content of the application referenced above is hereby incorporated by reference herein.

### STATEMENT REGARDING FEDERALLY SPONSORED RESEARCH

This invention was made with government support under CA213990 awarded by National Institutes of Health. The government has certain rights in the invention.

### BACKGROUND

The diagnosis of pancreatic ductal adenocarcinoma (PDAC) is a devastating one, with only 10% of patients surviving past 5 years. Although the survival rate since 2014 has increased from 6% to 10%, pancreatic cancer remains refractory to the majority of currently available therapeutics. In addition, as the demographics of the United States shifts, it is projected that pancreatic cancer will become the second leading cause of cancer-related mortality by 2030 and thus presents a significant future challenge for clinicians. Pancreatic cancer is particularly lethal due to the fact that in early stages there are seldom clinical symptoms, which results in 75-80% of patients being diagnosed with advanced, non-resectable disease. Even in patients who are eligible for resection, the 5-year survival rate is only 20-25%. Furthermore, pancreatic cancer has shown little responsiveness to immunotherapies which have shown remarkable effects in other solid tumors. The Phase I and II clinical trials using CTLA-4 and PD-1 inhibitors both alone and in combination have been deemed ineffective for the treatment of PDAC, which is likely explained by the non-immunogenic nature of PDAC.

A major challenge to the successful application of immunotherapy in PDAC is overcoming the immunosuppressive pancreatic tumor microenvironment (TME). PDAC is characterized by a dense pro-tumorigenic desmoplastic stroma, an abundance of immunosuppressive cell subsets within this stroma such as tumor-associated macrophages (TAMs), regulatory T-cells (T-regs), and myeloid-derived suppressor cells (MDSCs), a dearth of activated anti-tumor immune cells, and hypo-vascularity that lends to a hypoxic microenvironment. Together these conditions make it exceptionally challenging to effectively deliver immunotherapies to the pancreas and for these therapeutics to successfully activate anti-tumor immune responses if they arrive there. Therefore, novel therapeutics are desperately needed that can not only specifically target the pancreas, but that can also infiltrate the dense desmoplastic stroma, and that are capable of inciting robust anti-tumor immune responses despite the immunosuppressive TME.

Trained innate immunity is an evolutionarily ancient program of immunological memory that has recently come under in-depth scientific investigation. Trained innate immune cells have been shown to undergo transcriptomic, epigenetic and metabolic reprogramming upon exposure to specific initial stimuli, and when these innate immune cells are re-exposed to a secondary heterologous stimulus, they are “trained” to be more responsive to that stimulus which manifests in an enhanced inflammatory response. There are many biologics that are able to induce trained immunity including the Bacillus Calmette-Guerin (BCG) vaccine and the natural compound β-glucan. Though most studies regarding trained immunity focus on pathogens such as bacteria and viruses as a secondary stimulus, new studies suggest that tumor cells may also reactivate trained immune cells. All research to date has utilized subcutaneous models of cancer, therefore it is not known whether the presence of trained innate immune cells may invoke antitumor effects on tumors within solid organs, such as pancreatic cancer.

### SUMMARY

In one aspect, provided herein is a method of treating a pancreatic disorder comprising administering a therapeutically effective amount of yeast-derived particulate β-glucan. In one aspect, provided herein is a method inducing CCR2-dependent influx of immune cells to a pancreas comprising administering a therapeutically effective amount of yeast-derived particulate β-glucan. In one aspect, provided herein is a method of reducing tumor burden in a pancreas comprising administering a therapeutically effective amount of yeast-derived particulate β-glucan. In one aspect, provided herein is a method of recruiting anti-tumor, innate immune cells to pancreatic ductal adenocarcinoma (PDAC) tumor microenvironment (TME) comprising administering a therapeutically effective amount of yeast-derived particulate β-glucan. In one aspect, provided herein is a method of inducing whole β-Glucan particles (WGP)-induced trained immunity in a cancer comprising administering WGP. A method of inhibiting cancer metastasis comprising administering WGP. In one aspect, provided herein is a method of developing trained innate immune cells as an adoptive cell therapy in cancer.

### BRIEF DESCRIPTION OF DRAWINGS

FIGS. 1A-1E. Particulate β-glucan traffics to the pancreas in a dectin-1 dependent manner. (FIG. 1A) DTAF-WGP was injected i.p. and 3 days later different tissues were harvested and assessed for the presence of the DTAF-WGP by flow cytometry. (FIG. 1B) WGP was labeled with ⁸⁹Zr-WGP or (FIG. 1C) peritoneal macrophages were incubated with ⁸⁹Zr-WGP and washed, followed by I.P. injection. PET/CT imaging displays the trafficking of the ⁸⁹Zr-WGP after 48 hours. Organs were individually assessed for radioactivity following a necroscopy using a gamma counter. (FIG. 1D) Dectin-1^{-/-} mice or WT mice were injected with DTAF-WGP and the accumulation of DTAF-WGP in the pancreas was assessed by flow cytometry. (FIG. 1E) Dectin-1^{-/-} mice or WT mice were injected with ⁸⁹Zr-WGP and 48 hours later a PET/CT was used to assess the amount in the pancreas. The signal was quantified by reporting the % of injected dose (% ID) in the pancreas. Data are represented as mean±SEM. ns=not significant; *p<0.05, **p<0.01, ***p<0.001, ****p<0.0001.

FIGS. 2A-2K: β-glucan stimulates an influx of trained myeloid cells into the pancreas (FIG. 2A) WT mice were treated with I.P. DTAF-WGP and 3 days later the percent of CD11b⁺DTAF⁺ cells in the pancreas were identified with flow cytometry. Cells were previously gated on CD45⁺. (FIG. 2B) 7 days after I.P. PBS or WGP injection (7-day WGP), the percent of CD45⁺ cells in the pancreas were identified. The percent of CD11b⁺, CD3⁺, CD19⁺ and NK1.1⁺ cells within the CD45⁺ population were also measured. (FIG. 2C) Pie charts representing the relative change in frequency of each major immune cell population between the PBS vs 7-day WGP treatment setting. (FIG. 2D) WT mice were injected with WGP and the percent of CD45⁺ and CD45⁺CD11b^{+ c}ells were measured 7, 10, 18, and 30 days later. These were compared to PBS treated mice. (FIG. 2E) Gated on the pancreatic CD45⁺CD11b⁺ population, the percent of F4/80⁺ (macrophages), Ly6C⁺ (monocytes) and Ly6G⁺ (neutrophils) were measured using flow cytometry. (FIG. 2F) Mice were trained with PBS or WGP. Seven days later mouse pancreases were harvested and single-cell suspensions were restimulated with LPS. TNFα production in CD11b⁺, CD11b⁺F4/80⁺, and CD11b⁺Ly6C⁺ cells were measured using intracellular staining by flow cytometry. (FIG. 2G) Seven days after PBS or WGP training, the CD45⁺CD11b⁺ population was enriched using FACS sorting. Cells were plated and restimulated with PBS or LPS for 24 hours and the TNFα and IL-6 in the supernatant were measured using an ELISA. (FIG. 2H) Pancreatic CD11b⁺ cells from PBS and WGP-trained mice were sorted using FACS and RT-qPCR was done to quantify TNFα, IL-6, iNOS and IL-10 mRNA expression levels. (FIG. 2I) Mice were trained with PBS or WGP and 7 days later the CD45⁺CD1b⁺ populations were sorted and RNA-Seq analysis was performed (PBS n=3, WGP n=3). The distribution of p values (−log₁₀ (p value)) and fold changes (log₂ FC) of differentially expressed genes in WGP vs PBS trained cells are shown in a volcano plot. (FIG. 2J) Enrichment plots generated by Gene Set Enrichment Analysis (GSEA) for genes related to TNFα and IL-6 production, along with leukocyte chemotaxis and leukocyte migration in CD11b⁺ cells from 7-day WGP-trained as compared to PBS mice. (FIG. 2K) t-SNE plots of the CD11b⁺ population in mice trained with PBS or WGP 7 days prior and analyzed with CyTOF (PBS n=3, WGP n=3). Data are represented as mean±SEM. ns=not significant; *p<0.05, **p<0.01, ***p<0.001, ****p<0.0001.

FIGS. 3A-3K: Single-cell RNA-Seq showing the immune cell phenotype 3 and 7 days following IP WGP CD45⁺ cells from the pancreas of mice treated with PBS or WGP 7 days and 3 days prior were sorted and scRNA-Seq was performed. (FIG. 3A) Two dimensional UMAP representation of 11,132 cells aggregated from the three experimental samples with 20 unique clusters resulting from k-nearest neighbors and Louvain algorithms. (FIG. 3B) Heatmap of expression of aggregated marker genes for all clusters (FIG. 3C) Bar graphs showing the relative frequency of cells in each cluster across samples. (FIG. 3D) UMAP dimension reduction of PBS (left panel), 3-day WGP (middle panel) and 7-day WGP (right panel) samples shown individually. The portion of the UMAP representing myeloid cells is highlighted. (FIG. 3E) Single-cell gene expression of CSFIR. (FIG. 3F) Single-cell expression distributions across clusters identified as myeloid cells for select genes related to myeloid phenotyping. (FIG. 3G) Dot plot of the top 12 enriched genes in cluster 3,4,5 and 10 showing the average expression level and percentage of cells expressing select genes. (FIG. 3H) Dot plots showing the enrichment of selected genes associated with pro-inflammatory (first 25 genes) and anti-inflammatory (lasat 16 genes) immune responses across clusters 3,4,5 and 10. In G+H, average expression level is displayed as z-scores computed across the four clusters for individual genes.

FIGS. 4A-4K: CCR2 is required for immune cell trafficking into the pancreas (FIG. 4A) Heatmap of chemokines and cytokines that were upregulated in 7-day WGP treated CD11b⁺ cells based on RNA-Seq data. (FIG. 4B) viSNE plot of the CD11b⁺ pancreatic population in PBS and 7-day WGP-trained mice, highlighting the expression of CCR2. Images made with CyTOF data. (FIG. 4C) scRNA-Seq data showing a UMAP of the myeloid clusters expressing CCR2. (FIG. 4D) CCL2 expression in whole pancreatic lysates 24 hours following WGP treatment a measured by RT-PCR. (FIG. 4E) GSEA generated enrichment plots of genes related to leukocyte proliferation in CD11b⁺ pancreatic cells from 7-day WGP-trained as compared to PBS mice. (FIG. 4F) Summarized data of the percent of CD45⁺ pancreatic cells that are Ki67⁺ in PBS and 7-day WGP-trained mice. (FIG. 4G) Cells were first gated on the CD45⁺Ki67⁺ population. Plots show the percent of the CD45″Ki67″ proliferating pancreatic cells that are CD11b⁺CCR2⁺ in PBS and 7-day WGP-trained mice. (FIG. 4H) Pancreatic cells from PBS and 7-day WGP-trained mice were restimulated with LPS and the percent of CCR2 positive and negative cells producing TNFα was measured in each condition. Cells were first gated on the CD45⁺CD11b⁺ subset. (FIG. 4I-FIG. 4K) WT and CCR2^{-/-} mice were treated with PBS or WGP and the percent of (FIG. 4I) CD45⁺ cells in the pancreas (FIG. 4J) and the percent of CD45⁺ cells that were CD11b⁺ was assessed. (FIG. 4K) The percent of CD45⁺CD11b⁺ cells producing TNFα. Data are represented as mean±SEM. ns=not significant; *p<0.05, **p<0.01, ***p<0.001, ****p<0.0001.

FIGS. 5A-5F: WGP-trained pancreatic infiltrating myeloid cells show enhanced phagocytosis and ROS-mediated cytotoxicity (FIG. 5A) Enrichment plots (GSEA) and heat map of genes related to the positive regulation of phagocytosis in CD11b⁺ cells from 7-day WGP-trained as compared to PBS mice. (FIG. 5B) The percent of CD45⁺ pancreatic cells that phagocytosed a pHrodo Green Staph Aureus particle in PBS and 7day WGP mice along with the MFI of the pHrodo Green Staph Aureus particle. (FIG. 5C) The percent of CD11b⁺ myeloid pancreatic cells that phagocytosed a pHrodo Green Staph Aureus particle in PBS and 7-day WGP mice. Cells were first gated on the CD45⁺ population. The MFI of the pHrodo Green Staph Aureus particle is shown. (FIG. 5D) The percent of CD11b^{+ m}yeloid pancreatic cells that phagocytosed KPCGFP″ tumor cells in PBS and 7-day WGP mice. Cells were first gated on the CD45⁺ population. The MFI of the GFP⁺ signal is shown. (FIG. 5E) Enrichment plots (GSEA) and heat map of genes related to the reactive oxygen species biosynthetic processes and the positive regulation of reactive oxygen species metabolic processes in CD11b⁺ cells from 7-day WGP54 trained as compared to PBS mice. (FIG. 5F) Summarized results from a cytotoxicity assay where CD11b⁺ cells from PBS and 7-day WGP-trained mice were sorted and incubated at a ratio of 1:20 KPCLuc⁺:CD11b⁺ cells for 24 hours. NAC was used to block ROS expression and the tumor cytotoxicity was assessed by measuring luminescence (PBS n=3, WGP n=4). Data are represented as mean∓SEM. ns=not significant; *p<0.05, **p<0.01, ***p<0.001, ****p<0.0001.

FIGS. 6A-6I: The induction of trained immunity in the pancreas has potent anti-tumor effects (FIG. 6A) Experimental schema. (FIG. 6B) C57BL/6 mice received a single IP injection of WGP or PBS and 7 days mice were implanted orthotopically with KPC pancreatic cancer cells. Representative pictures of tumors and quantitative analysis of tumor weight are shown. Tumor weight was measured at day 21 (PBS n=5, WGP n=5). (FIG. 6C) C57BL/6 mice received a single i.p. injection of WGP or PBS and 7 days later were implanted orthotopically with KPC″Luc pancreatic cancer cells. On day 21 post tumor implantation, mice were given I.P. luciferin bioluminescent substrate and were placed in a photon imager to measure tumor size in vivo. (FIG. 6D) Survival of mice in the experimental schema shown in FIG. 6A, using KPC cells. (FIG. 6E) Phenotyping of the tumors showing the percent of viable cells that are CD45″, the percent of the CD45⁺ population that are CD11b⁺, and the percent of CD11b⁺ cells that are F4/80⁺. (FIG. 6F) TNFα production in CD11b⁺ cells from PBS and 7-day WGP-trained that were restimulated with LPS. Percent of TNFα⁺ cells and the MFI of TNFα are shown. (FIG. 6G) Summarized data of TNFα production in CD11b+F4/80⁺ cells from PBS and 7-dayWGP-trained that were restimulated with LPS. Percent of TNFα⁺ cells and the MFI of TNFα are shown. (FIG. 6H) Tumor weight was correlated with the percent of CD45⁺ immune cells that were CD11b⁺ (left) and the percent of CD45 CD11b⁺TNFα⁺ cells (right). (FIG. 6I) Summarized data of the percent of CD4⁺ and CD8⁺ T-Cells expressing IFNγData are represented as mean±SEM. Pearson correlation coefficients were used to measure the strength of the linear associations. ns=not significant; *p<0.05, **p<0.01, ***p<0.001, ****p<0.0001.

FIGS. 7A-7H: The anti-tumor effector mechanisms of WGP treatment and clinically relevant models (FIG. 7A) C57BL/6 were treated with PBS or WGP and CCR2^{-/-} mice were treated with WGP and 7 days later were implanted with orthotopic KPC pancreatic tumor cells. Tumor weight and size was monitored for 3 weeks after implantation and tumor weight at day 21 is reported. (WT PBS n=5, WT WGP n=5, CCR2^{-/-} WGP n=5). (FIG. 7B) Sorted CCR2⁺ and CCR2⁻ pancreatic myeloid cells from WGP trained mice were admixed with KPC cells and implanted orthotopically. Tumor size was evaluated 21 days later (CCR2⁺ n=5, CCR2⁻ n=5) (FIG. 7C) tSNE plots generated by CyTOF analysis of the CCR2⁺/CCR2⁺ admix tumors from FIG. 7B. Clusters that show significant differences between treatment groups are indicated by the circles. Total data (left) and representative images of each group (CCR2⁺-middle, CCR2⁻-right) are shown. (FIG. 7D) Summarized data for the percent of CD8⁺ and CD11b⁺ cells in CCR2⁺/CCR2⁻ admix tumors. (FIG. 7E) The ratio of CD8⁺ T-cells: CD11b⁺ myeloid cells in CCR2⁺ and CCR2⁻ admix tumors. (FIG. 7F) The expression of PD-L1 on KPC tumor cells, CD11b⁺ myeloid cells and F4/80⁺ macrophages in a KPC tumor 21 days after implantation (FIG. 7G) Experimental schema of WGP and anti PD-L1 therapy. Mice (n=5) were treated with PBS or WGP and 7 days later were implanted with orthotopic KPC pancreatic tumors. On days 3, 7 and 11 posttumor implantation, mice were given anti-PD-L1 mAb or anti-rat IgG2b mAb isotype control. Survival of mice was monitored. (FIG. 7H) Experimental schema of WGP used in the therapeutic setting. Mice were implanted with orthotopic KPC pancreatic tumors and were given WGP once mice had recovered from the surgery at day 4, and one week later on day 11. ns=not significant; *p<0.05, **p<0.01, ***p<0.001, ****p<0.0001.

FIGS. 8A-8I. WGP characterization and WGP-induced trained immunity in macrophages. (FIG. 8A) Topography image and PFIR images at 1040 cm⁻¹ of a dried WGP particle on silica substrate. (FIG. 8B) PFIR spectra scan at 4 different spots marked in the zoom-in topography image. (FIG. 8C) Stiffness and adhesion images of the same dried WGP particle. Scale bars: 2 μm; scale bars in zoom-in images: 500 nm. (FIG. 8D) Schema for WGP in vitro training assay (left). TNF-α production by in vitro WGP trained or untrained peritoneal macrophages after LPS re-stimulation assessed by ELISA (right). (FIG. 8E) TNF-α production by in vitro WGP-trained or untrained peritoneal macrophages upon co-culture with LLC and MLE-12 cells. (FIG. 8F) TNF-α production by in vitro WGP-trained or untrained peritoneal macrophages co-cultured with B16F10 (left) and EL-4 cells (right). (FIG. 8G) TNF-α production by in vitro WGP-trained or untrained peritoneal macrophages upon LLC or MLE-12 culture supernatant (sup) re-stimulation (left), and B16F10 or EL4 culture supernatant re-stimulation (right). (FIG. 8H) Levels of MIF from different cell culture supernatants measured by ELISA. (FIG. 8I) TNF-α production by in vitro WGP-trained or untrained peritoneal macrophages upon rMIF re-stimulation. Data are representative of two or three independent experiments and presented as mean±SEM. ns=not significant; **p<0.01, ****p<0.0001.

FIGS. 9A-9G. WGP in vivo treatment results in increased myeloid cells in the lung and trained phenotype in IM. C57B1/6 mice were injected with WGP (1 mg) or PBS intraperitoneally (i.p.) onday 0 and the lungs were harvested on day 7. Single cell suspensions were stained for analysis by flow cytometry. (FIG. 9A) Frequency of total viable CD45⁺ cells in the lungs. (FIG. 9B) Frequency of CD11b⁺ myeloid cells in the lungs. Cells were gated on viable CD45⁺ cells. (FIG. 9C) Frequency of AM and IM in the lungs. Cells were gated on viable CD45⁺ cells. (FIG. 9D) Frequency of inflammatory monocytes and patrolling monocytes in the lungs. Cells were gated on viable CD11b⁺ cells. (FIG. 9E-FIG. 9G) Percentage and mean fluorescence intensity (MFI) of intracellular TNF-α expression in lung IM and AM after ex vivo stimulation with LPS (FIG. 9E), LLC culture supernatants (FIG. 9F) or rMIF (FIG. 9G). Representative dot plots and summarized data are shown. Data are representative of two or three independent experiments and presented as mean±SEM. ns=not significant; *p<0.05, **p<0.01, ***p<0.001, ****p<0.0001.

FIGS. 10A-10F. WGP-induced trained response inhibits cancer metastasis. (FIG. 10A) Schema for in vivo WGP training and tumor challenge. (FIG. 10B) PBS vs WGP-trained mice were injected with 0.4×10⁶ LLC-GPF cells i.v. and tumor burden in the lungs were analyzed by flow cytometry. Representative dot plots and summarized percent of LLC-GFP cells in the CD45⁺ population in the lungs are shown (up). Histological analysis of the lungs from GFP-LLC tumor-bearing PBS vs WGP-trained mice (down). (FIG. 10C) Frequencies of LLC-GFP cells in the lungs from tumor-bearing PBS vs WGP-trained mice. Mice were trained with WGP on days −7, −14 and −21, respectively, prior to tumor challenge. (FIG. 10D) Long-term survival of PBS vs WGP-trained mice injected with 0.2×10⁶LLC-GFP cells i.v. on day 0. (FIG. 10E) Representative lung micrographs from PBS vs WGP-trained B16F10-tumor bearing mice. Black dots are melanoma lung metastasis nodules. Mice were trained at day −7, challenged with i.v. injections of 0.4×10⁶ B16F10 tumor cells at day 0 and the lungs harvested at day 16 (left). Long-term survival of PBS and WGP-trained B16F10 tumor cells (0.1×10⁶) challenged mice (right). (FIG. 10F) Mice were trained and challenged with 0.4×10⁶ EL4lymphoma cells i.v. at day 0 followed by euthanizing at day 16 or long-term survival observation. Representative liver micrographs, summarized number of liver nodules, liver weights, and long-term survival of PBS and WGP-trained EL4 tumor cell-bearing mice are shown. Data are representative of two or three independent experiments and presented as mean±SEM. *p<0.05, **p<0.01, ***p<0.001.

FIGS. 11A-11J. WGP-mediated training of lung IM prolongs overall survival in primary tumor-resected model and inhibits the development of tumor in a spontaneous lung adenocarcinoma model. (FIG. 11A) Schema for in vivo macrophage depletion by Clodrosome, WGP training, and tumor challenge. (FIG. 11B) Tumor burden in the lungs of PBS and WGP-trained versus WGP-trained-macrophage-depleted mice injected with 0.4×10⁶ LLC-GFP cells for 16 days. Representative dot plots and summarized data are shown. (FIG. 11C). Tumor burden in the lungs of PBS or WGP-trained versus neutrophil-depleted-WGP-trained mice. (FIG. 11D) Tumor burden in the lungs of PBS and WGP-trained versus WGP-trained CD4 T cell-depleted, WGP-trained CD8 T cell-depleted or WGP-trained CD4 and CD8 T cell-depleted mice. (FIG. 11E) Schema for 4T1 primary mammary tumor resection and WGP treatment protocol. Female Balb/c mice were implanted with 1×10⁶ 4T1 tumor cells on the fourth mammary pad. Tumors were surgically resected after a week and mice were treated with PBS or WGP three days after resection. Long-term survival was monitored. (FIG. 11F) Intracellular TNF-α expression on lung IM after ex vivo LPS re-stimulation of PBS versus WGP-trained Balb/c mice. (FIG. 11G) Long-term survival of PBS and WGP-trained 4T1 tumor resected Balb/c mice. (FIG. 11H) Schema for in vivo treatment of spontaneous K-ras^{LA1} mice. K-ras^{LA1} mice were i.p. injected with WGP or PBS at 6, 9, 12 and 15 weeks of age and euthanized at 17 weeks to analyze tumor development in the lungs. (FIG. 11I) Number of lung tumor nodules of PBS vs WGP-treated K-ras^{LA1} mice. Combined data from three independent experiments are shown. (FIG. 11J) Representative histology of lungs of PBS vs WGP-treated K-ras^{LA1} mice. Data are presented as mean±SEM. ns=not significant; *p<0.05, **p<0.01, ***p<0.001, ****p<0.0001.

FIGS. 12A-12G. WGP training results in an increased phagocytosis and mtROS-mediated cytotoxicity in lung IM. (FIG. 12A) Gene Set Enrichment Analysis (GSEA) plot for the regulation of phagocytosis (left) and heat-map for the genes related to the phagocytosis regulation pathway (right). (FIG. 12B) Phagocytosis assay was performed with lung AM and IM from WGP-trained or PBS control mice. Phagocytosis of pHrodo-green-labelled S. aureus was analyzed by flow cytometry. Representative dot plots and summarized data are shown. (FIG. 12C) In vitro cytotoxicity assay using sorted lung IM from PBS or WGP-trained mice and co-cultured with LLC cells at different ratios. Cells were cultured for 24 h and cytotoxicity was measured by LDH release assay. (FIG. 12D) In vivo cytotoxicity assay. PBS and WGP-trained mice were i.v. injected with 1×10⁶ LLC-GFP cells and were analyzed for the frequency of LLC-GFP cells in the lungs after 24 h. Representative dot plots and summarized data are shown. (FIG. 12E) GSEA plot for reactive oxygen species (ROS) biosynthetic process (left) and heatmap for the related leading genes in the WGP-trained lung IM (right). (FIG. 12F) MitoSox Red staining for PBS and WGP-treated peritoneal macrophages. Peritoneal macrophages were treated with PBS or WGP for 24 h and then stained with MitoSox Red and analyzed by flow cytometry. Representative histogram and summarized data from two independent experiments are shown. (FIG. 12G) Lung IM sorted from PBS vs WGP-trained mice were co-cultured with LLC target cells in the presence or absence of ROS inhibitor NAC at a 10:1 ratio. Cytotoxicity was measured by the LDH release assay. Data are representative of two or three independent experiments and presented as mean±SEM. ns=not significant; *p<0.05, **p<0.01, ****p<0.0001.

FIGS. 13A-13I. WGP treatment activates sphingolipid pathway resulting in an accumulation of S1P and subsequent trained phenotype in macrophages. (FIG. 13A) Heatmap for the genes upregulated in the sphingolipid synthesis pathway in the WGP-trained lung IM. (FIG. 13B) Detailed schema for the sphingolipid synthesis pathway (left) and qRT-PCR for CerS6 and Spkh2 mRNA expression in PBS and WGP-trained lung IM. (FIG. 13C) TNF-α production by WGP-trained or untrained peritoneal macrophages in the presence of Fumonisin-B1 (25 μM) or vehicle control DMSO. Peritoneal macrophages were trained with WGP in the presence of Fumonisin-B1 or DMSO for seven days and re-stimulated with LPS (left) or LLC culture supernatants (right). (FIG. 13D) TNF-α production by WGP-trained or untrained peritoneal macrophages in the presence of Sphk2i (25 μM or 50 μM) or DMSO upon LPS (left) or LLC culture supernatant (right) re-stimulation. (FIG. 13E) Representative mass spectrometry measurement of S1P in the in vitro WGP-trained or untrained peritoneal macrophages. One representative from three independent experiments with similar data. (FIG. 13F) TNF-α production by S1P-trained vs untrained peritoneal macrophages after LPS (left) or LLC culture supernatant (right) re-stimulation. (FIG. 13G) MitoSox Red staining on PBS vs S1P-trained peritoneal macrophages was analyzed by flow cytometry. Representative histogram and summarized data are shown. (FIG. 13H) The pDrp-1 expression in S1P-stimulated peritoneal macrophages assessed by flow cytometry (left) and WB analysis (right). (FIG. 13I) TNF-α production by S1P-trained vs untrained peritoneal macrophages in the presence of Mdivi-1 or vehicle control after LPS re-stimulation. Data are presented as mean±SEM. ns=not significant; *p<0.05, **p<0.01, ***p<0.001, ****p<0.0001.

FIGS. 14A-14GJ. Induction of mitochondrial fission by WGP treatment is required for the lung IM trained response and metastasis control. (FIG. 14A) Western blot analysis for p-Drp-1 and Drp-1 on peritoneal macrophages. Peritoneal macrophages were treated with PBS or WGP (50 μg/ml) in the presence of Sphk2i (50 μM) or DMSO for 3 h and 6 h. Cells were then harvested, lysed and the lysate was used for the detection of p-Drp-1 and total Drp-1 by Western blot. (FIG. 14B) Mitochondrialfission in PBS and WGP-trained versus WGP+Mdivi-1 (10 μM)-treated peritoneal macrophages. Peritoneal macrophages were stained with TMRM and analyzed by confocal microscopy (left). The lengths of mitochondrial fragments were analyzed by Image-J software (right). Representative confocal images and summarized data are shown. (FIG. 14C) TNF-α levels by PBS and WGP-trained peritoneal macrophages in the presence of Mdivi-1 (10 μM) or DMSO after LPS (left) and LLC culture supernatant (right) re-stimulation. (FIG. 14D) MitoSox Red staining on PBS vs WGP-trained peritoneal macrophages in the presence of Mdivi-1 (50 μM and 75 μM) using flow cytometry. Representative histogram and summarized data are shown. (FIG. 14E) Cytotoxicity of PBS vs WGP-trained peritoneal macrophages in the presence of Mdivi-1 (10 μM) or DMSO co-cultured with LLC target cells at a ratio of 10:1 using the LDH release assay. (FIG. 14F) Schema for Mdivi-1 in vivo treatment and tumor challenge. (FIG. 14G) Tumor burdens in the lungs from mice trained with or without WGP and treated with Mdivi-1 or vehicle control DMSO.

Representative dot plots and summarized data from two independent experiments are shown. (FIG. 14H, FIG. 14I) viSNE analysis of CyTOF immunophenotyping of the lungs from mice trained with or without WGP and treated with Mdivi-1 or vehicle control DMSO (n=4). All samples combined (FIG. 14H), combined samples from each group (FIG. 14I, top), and frequencies in different groups (FIG. 14I, bottom). (FIG. 14J) The ratios of CD8⁺ T cells to F4/80 CD11b⁺ and CD11b⁺PD-L1⁺ myeloid cells are shown. Data are presented as mean±SEM. ns=not significant; *p<0.05, **p<0.01, ***p<0.001, ****p<0.0001.

FIGS. 15A-15B. Adoptively transferred WGP-trained bone marrow-derived macrophages (BMDMs) elicit potent antitumor immunity to control metastasis. (FIG. 15A) Schema for in vitro WGP training in BMDMs and adoptive transfer protocol. BMDMs were trained with or without WGP in the presence of Mdivi-1 or vehicle control. Naïve mice received BMDMs on day 0 and day 2. On day 4, mice were challenged with LLC-GFP tumor cells. Mice were euthanized on day 21 post tumor challenge. (FIG. 15B) Lungs were harvested from mice that received BMDMs and tumor burden was assessed by flow cytometry. Representative dot plots and summarized data are shown. ****p<0.0001.

FIG. 16. Bioluminescence imaging (BLI) of NSG mice with orthotopic A549-luciferase tumor admixed with untrained or WGP-trained human CD14+ monocytes. Representative BLI images and summarized data are shown. *P<0.05.

### DETAILED DESCRIPTION

It was unexpectedly discovered that intraperitoneally (IP) injected yeast-derived particulate β-glucan traffics in large proportion to the pancreas. The direct trafficking of β-glucan to the pancreas highlights a novel pathway for the induction of peripheral trained immunity. Additionally, we tested the hypothesis that this trafficking might result in the induction of antitumor immunity against pancreatic cancer. Given the failure of the majority of therapeutics and immunotherapies alone and in combination to treat PDAC and the difficulty of targeting these therapeutics to the pancreas, our findings collectively provide a potential breakthrough in not only targeting the pancreas directly, but also in recruiting anti-tumor, innate immune cells to the immunologically cold PDAC TME.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer.

To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Gastric cancer, as used herein, includes stomach cancer, which can develop in any part of the stomach and may spread throughout the stomach and to other organs; particularly the esophagus, lungs, lymph nodes, and the liver.

A “chemotherapeutic agent” is a biological (large molecule) or chemical (small molecule) compound useful in the treatment of cancer, regardless of mechanism of action. Classes of chemotherapeutic agents include, but are not limited to alkylating agents, antimetabolites, spindle poison plant alkaloids, cytotoxic/antitumor antibiotics, topoisomerase inhibitors, proteins, antibodies, photosensitizers, and kinase inhibitors. Chemotherapeutic agents include compounds used in “targeted therapy” and non-targeted conventional chemotherapy.

The term “mammal” includes, but is not limited to, humans, mice, rats, guinea pigs, monkeys, dogs, cats, horses, cows, pigs, sheep, and poultry.

The term “package insert” is used to refer to instructions customarily included in commercial packages of therapeutic products, that contain information about the indications, usage, dosage, administration, contraindications and/or warnings concerning the use of such therapeutic products.

The term “synergistic” as used herein refers to a therapeutic combination which is more effective than the additive effects of the two or more single agents. A determination of a synergistic interaction between a compound of formula I or a pharmaceutically acceptable salt thereof and one or more chemotherapeutic agent may be based on the results obtained from the assays described herein. The results of these assays can be analyzed using the Chou and Talalay combination method and Dose-Effect Analysis with CalcuSyn software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents. The program utilized is that described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 to 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. In some examples,

Combination effects were evaluated using both the BLISS independence model and the highest single agent (HSA) model (Lehár et al. 2007, Molecular Systems Biology 3:80). BLISS scores quantify degree of potentiation from single agents and a BLISS score>0 suggests greater than simple additivity. An HSA score>0 suggests a combination effect greater than the maximum of the single agent responses at corresponding concentrations.

In one aspect, the invention provides a method of treating a pancreatic disorder comprising administering a therapeutically effective amount of a therapeutic agent comprising a yeast-derived particulate β-glucan. In one aspect, the pancreatic disorder is a cancer. In one aspect, the cancer is pancreatic ductal adenocarcinoma (PDAC).

In one aspect, the invention provides a method inducing CCR2-dependent influx of immune cells to a pancreas comprising administering a therapeutically effective amount of a therapeutic agent comprising a yeast-derived particulate β-glucan. In one aspect, the immune cells are monocytes or macrophages.

In one aspect, the invention provides a method of reducing tumor burden in a pancreas comprising administering a therapeutically effective amount of a therapeutic agent comprising a yeast-derived particulate β-glucan.

In one aspect, the invention provides a method of recruiting anti-tumor, innate immune cells to pancreatic ductal adenocarcinoma (PDAC) tumor microenvironment (TME) comprising administering a therapeutically effective amount of a therapeutic agent comprising a yeast-derived particulate β-glucan.

In one aspect, the invention provides a method of inducing whole β-Glucan particles (WGP)-induced trained immunity in a cancer comprising administering a therapeutic agent comprising a WGP. In one aspect, the cancer is pancreatic cancer. In one aspect, the cancer is lung cancer.

In one aspect, the invention provides a method of inhibiting cancer metastasis comprising administering a therapeutic agent comprising a WGP.

In one aspect, the yeast-derived particulate β-glucan comprises whole β-Glucan particles (WGP). In certain aspects, the yeast-derived whole β-glucan particles (WGP) are micro-particles of 1,3-β-glucan extracted from the yeast Saccharomyces cerevisiae, which have been shown to activate immune cells through the stimulation of C-type lectin receptor, dectin-1.

In one aspect, the method further comprises administering an anti-Programmed Death ligand-1 (anti-PD-L1) immunotherapy. In one aspect, the anti-PD-L1 immunotherapy is an anti-PD-L1 monoclonal antibody (mAb) therapy.

In one aspect, the method further comprises administering anti-Programmed Death-1 (PD-1) or anti-CTLA-4 immunotherapy. In one aspect, the anti-PD-1 or anti-CTLA-4 immunotherapy is an anti-PD-1 or anti-CTLA-4 mAb therapy.

In one aspect, the yeast-derived particulate β-glucan is derived from Saccharomyces cerevisiae.

In one aspect, the yeast-derived particulate β-glucan is in the form of whole β-glucan particles (WGP) derived from Saccharomyces cerevisiae.

In one aspect, the WGP comprise 2-4 micron hollow yeast cells made of highly concentrated (1,3) β-glucans.

In one aspect, the yeast-derived particulate β-glucan is administered by means of injection.

In one aspect, the injection is an intraperitoneal injection.

In one aspect, the injection is an intratumoral injection

In one aspect, the yeast-derived particulate β-glucan is administered orally.

In one aspect, provided herein is an isolated or purified beta-glucan-trained innate immune cell.

In one aspect, provided herein is a method of producing a composition for adoptive cell therapy in cancer comprising contacting in vitro or ex vivo an innate immune cell with yeast-derived particulate β-glucan, and culturing the innate immune cell to generate a beta-glucan-trained innate immune cell.

In one aspect, the yeast-derived particulate β-glucan is in the form of whole β-glucan particles (WGP)

In one aspect, the WGP is derived from Saccharomyces cerevisiae. In one aspect, the WGP comprise 2-4 micron hollow yeast cells made of highly concentrated (1,3) β-glucans.

In one aspect, provided herein is a beta-glucan-trained innate immune cell made by the method described herein.

In one aspect, provided herein is a method of developing trained innate immune cells as an adoptive cell therapy in cancer.

In one aspect, provided herein is an in vitro or ex vivo beta-glucan-trained innate immune cell as an adoptive cell therapy in cancer.

In one aspect, the method further comprises administering an anti-Programmed Death ligand-1 (anti-PD-L1) immunotherapy. In one aspect, the anti-PD-L1 immunotherapy is an anti-PD-L1 monoclonal antibody (mAb) therapy.

In one aspect, the method further comprises administering anti-Programmed Death-1 (PD-1) or anti-CTLA-4 immunotherapy. In one aspect, the anti-PD-1 or anti-CTLA-4 immunotherapy is an anti-PD-1 or anti-CTLA-4 mAb therapy.

In one aspect, the method further comprises administering anti-CD47 or anti-SIRPalpha immunotherapy. In one aspect, the anti-CD47 or anti-SIRPalpha immunotherapy is an anti-CD47 or anti-SIRPalpha mAb therapy.

Immunotherapies Certain embodiments of the present invention provide an immune reagent comprising an antibody. As used herein, the term “antibody” includes scFv, humanized, fully human or chimeric antibodies, single-chain antibodies, diabodies, and antigen-binding fragments of antibodies that do not contain the Fc region (e.g., Fab fragments). In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

As used herein, the term “antibody” includes a single-chain variable fragment (scFv or “nanobody”), humanized, fully human or chimeric antibodies, full length antibodies, single-chain antibodies, diabodies, and antigen-binding fragments of antibodies (e.g., Fab fragments). Dondelinger et al., “Understanding the Significance and Implications of Antibody Numbering and Antigen-Binding Surface/Residue Definition,” Frontiers in Immunology, Vol. 9, Article 2278 (18 Oct. 2018). A scFv is a fusion protein of the variable region of the heavy (V_{H}) and light chains (V_{L}) of an immunoglobulin that is connected by means of a linker. In certain embodiments, the linker between the V_{H} and V_{L} is a peptide. In certain embodiments, the linker is short, about 3-25 amino acids in length. In certain embodiments the linker is about 3-12 amino acids in length. If flexibility is important, the linker will contain a significant number of glycines. If solubility is important, serines or threonines will be utilized in the linker. The linker may link the amino-terminus of the V_{H} to the carboxy-terminus of the V_{L}, or the linker may link the carboxy-terminus of the V_{H} to the amino-terminus of the V_{L}. Divalent (also called bivalent) scFvs can be generated by linking two scFvs. For example, a divalent scFv can be made by generating a single peptide containing two V_{H} and two V_{L} regions. Alternatively, two peptides, each containing a single V_{H} and a single V_{L} region can be dimerized (also called “diabodies”). In certain embodiments, the linker that is used to link the two scFv moieties is a peptide. In certain embodiments, the linker is short, about 3-25 amino acids in length.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods. The monoclonal antibodies used in the present invention can be also isolated from a phage antibody library. The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention.

Monoclonal antibodies can be prepared by methods known to those skilled in the art.

### Formulations and Methods of Administration

In certain embodiments, an effective amount of the therapeutic composition is administered to the subject. “Effective amount” or “therapeutically effective amount” are used interchangeably herein, and refer to an amount of a compound, formulation, material, or composition, as described herein effective to achieve a particular biological result. Such results may include, but are not limited to the treatment of cancer as determined by any means suitable in the art.

In certain embodiments, the therapeutic composition is administered via intramuscular, intradermal, or subcutaneous delivery. In certain embodiments, the therapeutic composition is administered via a mucosal surface, such as an oral, or intranasal surface. In certain embodiments, the therapeutic composition is administered via intrasternal injection, or by using infusion techniques.

In certain embodiments, “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The vaccines and compositions of the invention may be formulated as pharmaceutical compositions and administered to a mammalian host, such as a human patient, in a variety of forms adapted to the chosen route of administration, i.e., orally, intranasally, intradermally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions. For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Additional ingredients such as fragrances or antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Formulations will contain an effective amount of the active ingredient in a vehicle, the effective amount being readily determined by one skilled in the art. “Effective amount” is meant to indicate the quantity of a compound necessary or sufficient to realize a desired biologic effect. The active ingredient may typically range from about 1% to about 95% (w/w) of the composition, or even higher or lower if appropriate. The amount for any particular application can vary depending on such factors as the severity of the condition. The quantity to be administered depends upon factors such as the age, weight and physical condition of the animal considered for vaccination and kind of concurrent treatment, if any. The quantity also depends upon the capacity of the animal's immune system to synthesize antibodies, and the degree of protection desired. Typically, dosages used in vitro may provide useful guidance in the amounts useful for in situ administration of the composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Reminpton's Pharmaceutical Sciences, 17th ed., Mack Publishing Co., Easton, Pa., 1990, each of which is herein incorporated by reference. Additionally, effective dosages can be readily established by one of ordinary skill in the art through routine trials establishing dose response curves. The subject is immunized by administration of the composition thereof in one or more doses. Multiple doses may be administered as is required to maintain a state of immunity to the target. For example, the initial dose may be followed up with a booster dosage after a period of about four weeks to enhance the immunogenic response. Further booster dosages may also be administered. The composition may be administered multiple (e.g., 2, 3, 4 or 5) times at an interval of, e.g., about 1, 2, 3, 4, 5, 6 or 7, 14, or 21 days apart.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

Thus, the present compositions may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, the present compositions may be combined with one or more excipients and used in the form of ingestible tablets, buccal tablets, troches, capsules, elixirs, suspensions, syrups, wafers, and the like. Such preparations should contain at least 0.1% of the present composition. The percentage of the compositions may, of course, be varied and may conveniently be between about 2 to about 60% of the weight of a given unit dosage form. The amount of present composition in such therapeutically useful preparations is such that an effective dosage level will be obtained.

Useful dosages of the compositions of the present invention can be determined by comparing their in vitro activity, and in vivo activity in animal models. The amount of the compositions described herein required for use in treatment will vary with the route of administration and the age and condition of the subject and will be ultimately at the discretion of the attendant veterinarian or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations will be dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

Sustained-release preparations of Formula I compounds may be prepared. Suitable examples of sustained-release preparations include semipermeable matrices of solid hydrophobic polymers containing a compound of Formula I, which matrices are in the form of shaped articles, e.g., films, or microcapsules. Examples of sustained-release matrices include polyesters, hydrogels (for example, poly (2-hydroxyethyl-methacrylate), or poly (vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D (−)3-hydroxybutyric acid.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono-or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight: weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The therapeutic agent may be employed in combination with other chemotherapeutic agents for the treatment of a hyperproliferative disease or disorder, including tumors, cancers, and neoplastic tissue, along with pre-malignant and non-neoplastic or non-malignant hyperproliferative disorders. In certain embodiments, a therapeutic agent is combined in a dosing regimen as combination therapy, with a second compound that has anti-hyperproliferative properties or that is useful for treating the hyperproliferative disorder. The second compound of the dosing regimen preferably has complementary activities to the therapeutic agent, and such that they do not adversely affect each other. Such therapeutic agents may be administered in amounts that are effective for the purpose intended. In one embodiment, the therapeutic combination is administered by a dosing regimen wherein the therapeutically effective amount of a therapeutic agent is administered in a range from twice daily to once every three weeks (q3wk), and the therapeutically effective amount of the chemotherapeutic agent is administered in a range from twice daily to once every three weeks.

The combination therapy may be administered as a simultaneous or sequential regimen. When administered sequentially, the combination may be administered in two or more administrations. The combined administration includes coadministration, using separate formulation, and consecutive administration in either order, wherein preferably there is a time period while both (or all) active agents simultaneously exert their biological activities.

In one specific aspect of the invention, the therapeutic agent can be administered for a time period of about 1 to about 10 days after administration of the one or more agents begins. In another specific aspect of the invention, the therapeutic agent can be administered for a time period of about 1 to 10 days before administration of the combination begins. In another specific aspect of the invention, administration of the therapeutic agent and administration of the chemotherapeutic agent begin on the same day.

Suitable dosages for any of the above coadministered agents are those presently used and may be lowered due to the combined action (synergy) of the newly identified agent and other chemotherapeutic agents or treatments, such as to increase the therapeutic index or mitigate toxicity or other side-effects or consequences.

In a particular embodiment of anti-cancer therapy, a therapeutic agent, may be combined with a chemotherapeutic agent, as well as combined with surgical therapy and radiotherapy. The amounts of the therapeutic agent and the other pharmaceutically active chemotherapeutic agent(s) and the relative timings of administration will be selected in order to achieve the desired combined therapeutic effect.

### Administration of Pharmaceutical Compositions

The compounds may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. Formulation of drugs is discussed in Remington's Pharmaceutical Sciences, 18^{th} Ed., (1995) Mack Publishing Co., Easton, PA. Other examples of drug formulations can be found in Liberman, H. A. and Lachman, L., Eds., Pharmaceutical Dosage Forms, Marcel Decker, Vol 3, 2^{nd} Ed., New York, NY. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier, glidant, or excipient. Where the compound is administered parenterally, it may be formulated with a pharmaceutically acceptable parenteral vehicle or diluent, and in a unit dosage injectable form, as detailed below.

A dose to treat human patients may range from about 20 mg to about 1600 mg per day of the therapeutic agent. A typical dose may be about 50 mg to about 800 mg of the compound. A dose may be administered once a day (QD), twice per day (BID), or more frequently, depending on the pharmacokinetic (PK) and pharmacodynamic (PD) properties, including absorption, distribution, metabolism, and excretion of the particular compound. In addition, toxicity factors may influence the dosage and administration dosing regimen. When administered orally, the pill, capsule, or tablet may be ingested twice daily, daily or less frequently such as weekly or once every two or three weeks for a specified period of time. The regimen may be repeated for a number of cycles of therapy.

### Articles of Manufacture

In another embodiment of the invention, an article of manufacture, or “kit”, containing a therapeutic agent useful for the treatment of the diseases and disorders described above is provided. In one embodiment, the kit comprises a container and a therapeutic agent.

The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a therapeutic agent, or a formulation thereof which is effective for treating the condition and may have a sterile access port (for example, the container may be an intravenous solution bag or a vial having a stopper pierceable by a hypodermic injection needle). At least one active agent in the composition is a therapeutic agent. The label or package insert indicates that the composition is used for treating the condition of choice, such as cancer. In one embodiment, the label or package inserts indicates that the composition comprising a therapeutic agent can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of a therapeutic agent, and, if present, the second pharmaceutical formulation. For example, if the kit comprises a first composition comprising a therapeutic agent and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a therapeutic agent, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a therapeutic agent contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of a therapeutic agent and a second therapeutic agent, i.e. the chemotherapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

The invention will now be illustrated by the following non-limiting Examples.

### EXAMPLE 1

### Induction of Peripheral Trained Immunity in the Pancreas Incites Anti-tumor Activity to Control Pancreatic Cancer Progression

### Summary

Despite the remarkable success of immunotherapy in many types of cancer, pancreatic ductal adenocarcinoma (PDAC) has yet to benefit. Innate immune cells are critical to antitumor immunosurveillance and recent studies have revealed that these populations possess a form of memory, termed trained innate immunity, which occurs through transcriptomic, epigenetic, and metabolic reprograming. Though trained innate immunity has mostly been investigated in the context of infection, the induction of trained innate immunity could also protect against tumors.

It has been demonstrated that yeast-derived particulate β-glucan, an inducer of trained immunity, unexpectedly traffics to the pancreas. This causes a robust CCR2-dependent influx of newly characterized monocytes/macrophages to the pancreas which display features of trained immunity. These trained cells can be activated upon exposure to tumor cells and tumor-derived factors, and show enhanced phagocytosis and ROS-mediated cytotoxicity against pancreatic tumor cells. In orthotopic models of PDAC, mice trained with β-glucan show significantly reduced tumor burden and prolonged survival, which is further enhanced when combined with anti-PD-L1 immunotherapy. Collectively, these findings not only add significant novel characterization to the dynamic mechanisms, scope and localization of peripheral trained immunity, but also identify a direct application of trained immunity to cancer that can be utilized within the pancreas to reprogram the hostile tumor microenvironment of PDAC.

### Results

### Yeast-Derived Particulate β-Glucan Preferentially Traffics to the Pancreas

It has been well documented that administration of β-glucans from bacteria and fungi results in an increase in the numbers and frequencies of hematopoietic progenitors and multipotent progenitors that are biased towards the myeloid lineage, which ultimately functions as an important step in the induction of central and peripheral trained immunity. However, it has not yet been shown where exactly β-glucan traffics after administration to have these effects. In these studies, whole β-Glucan particles (WGP) derived from Saccharomyces cerevisiae were used, which are 2-4 micron hollow yeast cells made of highly concentrated (1,3) β-glucans. The detailed characterization of WGP β-glucan is described in one of our other studies. Unlike other forms of β-glucan used in trained immunity, this formulation is unique in that it is a particulate and requires active phagocytosis to exert its effects.

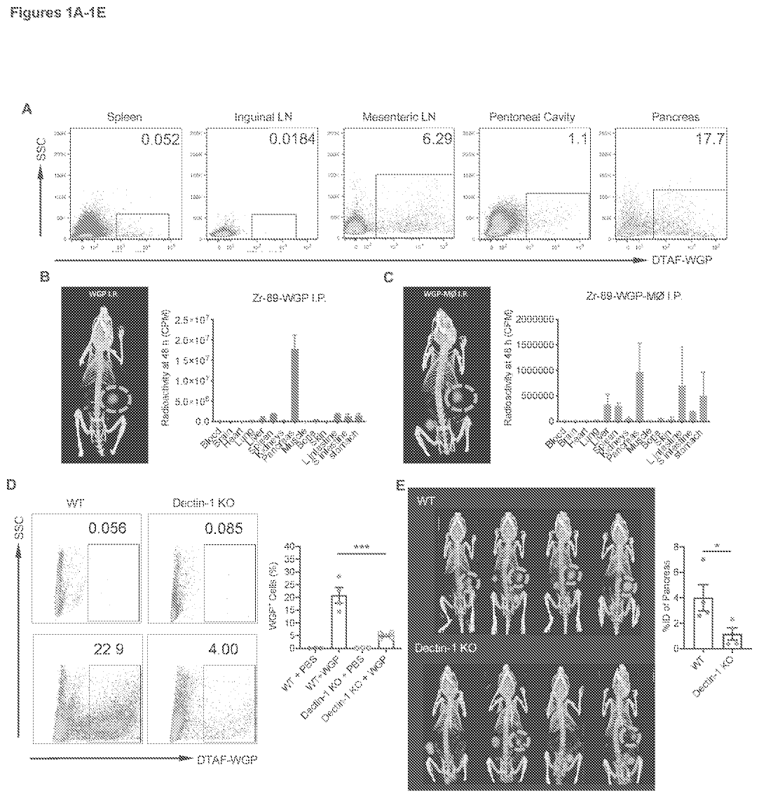

To assess the trafficking of this particulate β-glucan, WGP was tagged with (5-(4,6-Dichlorotriazinyl) Aminofluorescein) (DTAF), and injected IP into wildtype (WT) C57BL/6 mice. Three days following IP administration (3-day WGP), the lung, spleen, inguinal and mesenteric lymph nodes, peritoneal cavity cells, and pancreas were harvested to detect the presence of DTAF-WGP. While there was some trafficking of the DTAF-WGP to the spleen, mesenteric lymph nodes, and residual DTAF-WGP in the peritoneal cavity, the pancreas showed a striking and unexpected presence of the DTAF-WGP (FIG. 1A). To further assess this trafficking and to ensure that the DTAF label was not involved in the trafficking mechanism, WGP was radiolabeled with ⁸⁹Zr and injected IP (FIG. 1B) or incubated with peritoneal macrophages that were then injected IP (FIG. 1C). Mice were first imaged using a PET/CT scan 48 hours following injection and green circles are used to indicate the observed preferential accumulation of the ⁸⁹Zr-WGP in the pancreas. A necroscopy was then performed, and the radioactive signature of each organ was measured. In accordance with the flow cytometry data, ⁸⁹Zr-WGP trafficked in large quantities to the pancreas, and was found in lower levels in the spleen, liver and intestinal system. Peritoneal macrophages that were cultured with ⁸⁹Zr-WGP and then injected IP had similar though slightly more diversified trafficking than the pure ⁸⁹Zr-WGP, and also accumulated most prominently in the pancreas (FIG. 1D). Trafficking of peritoneal macrophages loaded with WGP indicates that both naked WGP as well as macrophages that have phagocytosed WGP display tropism to the pancreas.

In an effort to assess the role that the known receptor of WGP played in the trafficking, mice lacking the C-type lectin receptor, Dectin-1, (Dectin-1^{-/-} mice) were injected with DTAF-WGP. As compared to WT mice, Dectin-1^{-/-} mice showed a 5-fold decrease in the amount of WGP that trafficked to the pancreas, as assessed by flow cytometry (FIG. 1D). ⁸⁹Zr-WGP was also injected IP into WT mice and Dectin-1^{-/-} mice. As compared to WT animals, there was significantly less trafficking of WGP to the pancreas of Dectin-1^{-/-} mice (FIG. 1E). To ensure that this process was in fact β-glucan specific, a polystyrene-based latex 3 μm fluorescent particle, the same size as a WGP particle, was injected IP and was not found to accumulate in the pancreas. Together, these data highlight a previously uncharacterized dectin-1 dependent tropism of β-glucan trafficking to the pancreas.

β-Glucan that Traffics to the Pancreas Incites an Influx of Innate Immune Cells that Show a Phenotype of Trained Immunity

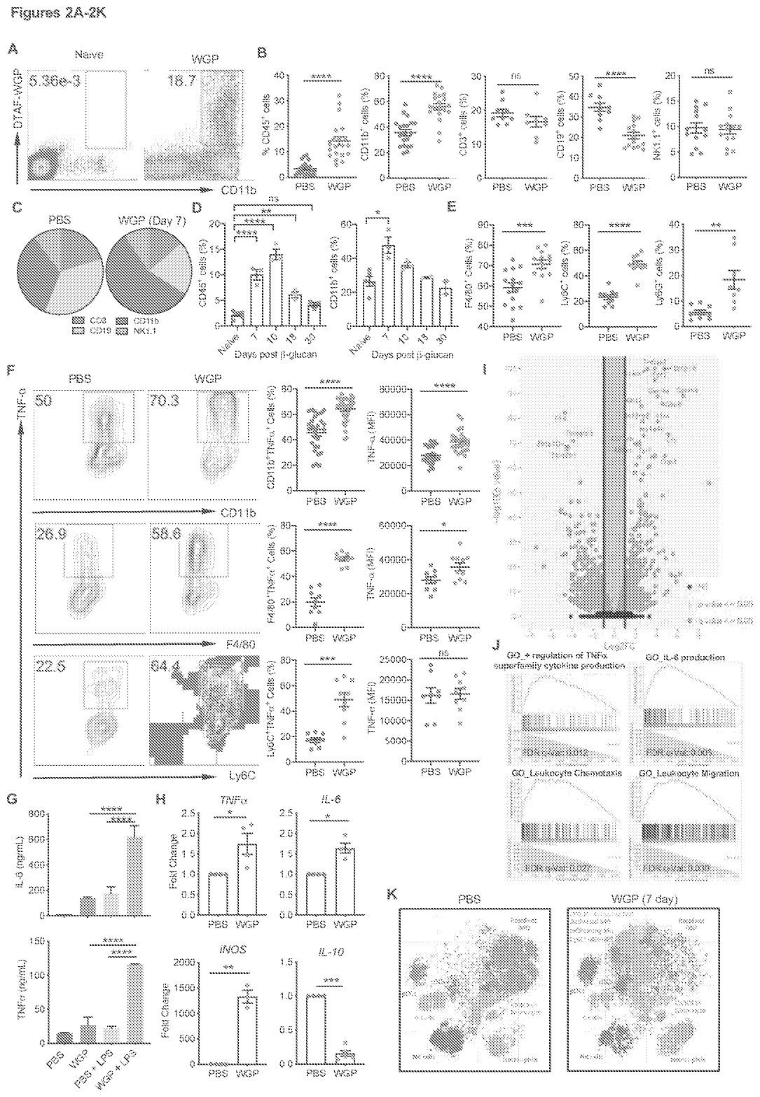

After discovering that β-glucan displays tropism toward the pancreas, it was also observed that the immune landscape of the pancreas was significantly altered following WGP treatment. Arrival of WGP to the pancreas was accompanied by a distinct influx of CD11b⁺ myeloid cells to the pancreas by day 3, some of which had phagocytosed WGP (FIG. 2A). This finding led us to examine how overall immune populations of the pancreas are impacted following IP WGP treatment. As it has been previously observed that the immune changes associated with trained immunity in the BM are most pronounced one week following exposure to β-glucan, the immune profile of the pancreas was examined in mice treated with a 3 μm polystyrene microparticle or WGP. First, it was observed that there was about a 10-fold increase in the overall percent of CD45⁺ immune cells in the pancreas after WGP treatment (FIG. 2B). The 3 μm polystyrene microparticle control did not have such effects, indicating that this immune cell influx is WGP specific. In addition, there was an observed increase in the absolute number of CD45⁺ immune cells. To further classify which cell types were responsible for this expansion of the CD45⁺ immune population, the relative percent (FIG. 2B) and absolute number of myeloid cells (CD11b⁺), T-cells (CD3⁺), β-cells (CD19⁺), and NK cells (NK1.1⁺ ) were evaluated. The cumulative changes in the pancreas are represented by pie charts which demonstrate that the expansion of the myeloid compartment is responsible for the relative decrease in the percentage of other cell populations and the overall increase in the CD45⁺ population following WGP treatment (FIG. 2C).

We next investigated whether this influx of CD11b⁺ immune cells was transient, so the percent of overall CD45⁺ cells and CD11b⁺ cells in the pancreas following IP injection was measured at 7, 10, 18 and 30 days. While the percent of CD11b⁺ cells peaked at day 7, the percent of CD45⁺ cells appeared to peak at day 10 which may indicate that following influx of CD11b⁺ cells to the pancreas, an influx of other immune cell types may follow. The percent of immune cells in the pancreas decreased to basal levels by day 30, demonstrating that this influx is transient (FIG. 2D). Within the myeloid compartment, we observed that the percent (FIG. 2E) and absolute numbers of macrophages (F4/80⁺), monocytes (Ly6C⁺), and neutrophils (Ly6G⁺) were all increased. The influx of immune cells into the pancreas was shown to be dependent on the dose of WGP injected, where higher doses of WGP were associated with increased influx of overall CD45⁺CD11b⁺ myeloid cells and CD45⁺CD11b⁺F4/80⁺ macrophages. As pancreatitis and the destruction of pancreatic islets are associated with immune cell infiltration of the pancreas, H+E of the pancreas was performed to assess the integrity of acini 7 days after WGP administration. Serum amylase, a diagnostic marker of pancreatitis, was also measured in WGP vs PBS-treated mice 7 days following injection. Neither the islets nor the serum amylase was adversely impacted by WGP treatment and the observed immune influx, indicating that the immune cell influx in this mechanism does not cause pancreatic destruction. Further, following WGP treatment mice were monitored for up to 3 months, and no morbidities or mortalities were associated with WGP treatment.

To investigate whether the cells found to infiltrate the mouse pancreas following WGP treatment displayed a phenotype of trained immunity, we used a standard training protocol in which mice were treated with WGP or PBS and 7 days later the pancreas were harvested and then restimulated with LPS. TNF-α production has been used as a surrogate marker to evaluate the trained response. Overall CD11b⁺ myeloid cells, CD11b^{+F}4/80⁺ macrophages and CD11b⁺Ly6C⁺ monocytes were all shown to produce more TNF-α due to prior exposure to WGP, as assessed by the percent of TNF-α⁺ cells and the MFI (FIG. 2F). To further assess these findings, CD11b⁺ cells were sorted from the pancreas of these mice and restimulated ex-vivo with LPS, and the TNF-α and IL-6 levels in the supernatants were measured by ELISA. As compared to CD11b⁺ cells from PBS mice, cells from WGP-trained mice that were restimulated with LPS produced significantly more TNF-α and IL-6 (FIG. 2G), signifying that WGP treated pancreatic CD11b⁺ cells were trained. These results were further confirmed using RT-PCR, where pancreatic CD11b⁺ cells sorted from PBS or WGP-trained mice were shown to produce more TNF-α, IL-6, iNOS and less IL-10 due to WGP treatment (FIG. 2H). Together these findings indicate that not only does WGP incite a trained phenotype in the cells that traffic into the pancreas, but that they also have an overall proinflammatory phenotype.

RNA sequencing (RNA-Seq) was then performed on FACS sorted CD45⁺CD11b^{+ cells} 7 days following PBS or WGP training to obtain an unbiased and comprehensive characterization of myeloid populations in the pancreas. A total of 1459 Differentially Expressed Genes (DEGs) were discovered, with 661 upregulated and 798 genes downregulated in the WGP-trained setting (FIG. 2I). Gene set enrichment analyses (GSEA) further confirmed previously investigated upregulations in pathways related to TNF-α cytokine production, IL-6 production, and nitric oxide production (FIG. 2J). In accordance with the observed influx of immune cells, the DEGs related toleukocyte chemotaxis, leukocyte migration (FIG. 2J), monocyte chemotaxis, macrophage migration, mononuclear cell migration, and myeloid leukocyte migration were all significantly enriched. Additionally, Reactome pathways CLEC7A dectin-1 signaling and C-typelectin receptor pathways were significantly enriched, further corroborating the Dectin-1 involvement in this process.

Considering that the myeloid population was observed to be responsible for the expansion of the CD45⁺ population and that myeloid cells are a diverse population, CyTOF analysis was performed on pancreata from mice treated with PBS or WGP seven days prior. Cell populations within the pancreas were identified and compared between treatment groups; t-SNE plots indicated that following WGP treatment there was a relative decrease in the resident macrophage population which highly expresses M2 markers such as CD206. We also observed an appearance of several myeloid populations in the pancreas which were defined as Ly6C-macrophage-derived monocytes, infiltrating inflammatory monocytes, activated macrophages, and CD206⁺ activated macrophages. (FIG. 2K). viSNE plots gated on the CD11b⁺ population showed a distinct expansion of monocytes and macrophages, with additional increases of the Dendritic Cell (DCs), neutrophil, and NK populations after WGP treatment. Additionally, these analyses highlight that following WGP training and LPS restimulation, TNFα was primarily produced by macrophages and monocytes, IL-6 and iNOS were primarily produced by macrophages, Granzyme-B was produced by NK-cells and macrophages, and IFNg was principally produced by neutrophils. This reveals that while many cell populations are changed due to WGP treatment, the primary cells trained by WGP, as assessed by increased TNFα expression, are macrophages and monocytes and that there is not a single cell type responsible for the phenotype of the trained immunity that was observed.

To assess whether this phenomenon of myeloid cell influx and activation was dependent upon other immune populations or driven entirely by the myeloid cells themselves, we depleted specific immune populations and observed the trained phenotype. Anti-CD4 and anti-CD8 mAbs were used alone and in combination to deplete T-cells, the efficiency of depletion was assessed, and the trained phenotype of myeloid cells was observed. NK cells were depleted using mAb PK 136, the depletion efficiency was evaluated, and the trained phenotype was observed. In the absence of T-cells and NK cells, myeloid cells were still observed to be trained by WGP. The trained phenotype was also assessed in NSG mice which lack β-cells, T-cells, and NK cells. These mice still showed a clear phenotype of trained immunity. We also depleted neutrophils using Ly6G mAb. Despite depletion of granulocytes, we still observed WGP-induced training in the CD11b⁺ myeloid compartment. Taken together, our data support that the infiltrating myeloid cells in the pancreas are trained by β-glucan without the assistance or influence of adaptive immune populations, NK cells, or granulocytes.

A Single-Cell RNA Sequencing Reveals Specific Populations of Pro-Inflammatory Macrophage/Monocytes that Traffic to the Pancreas Upon β-Glucan Treatment

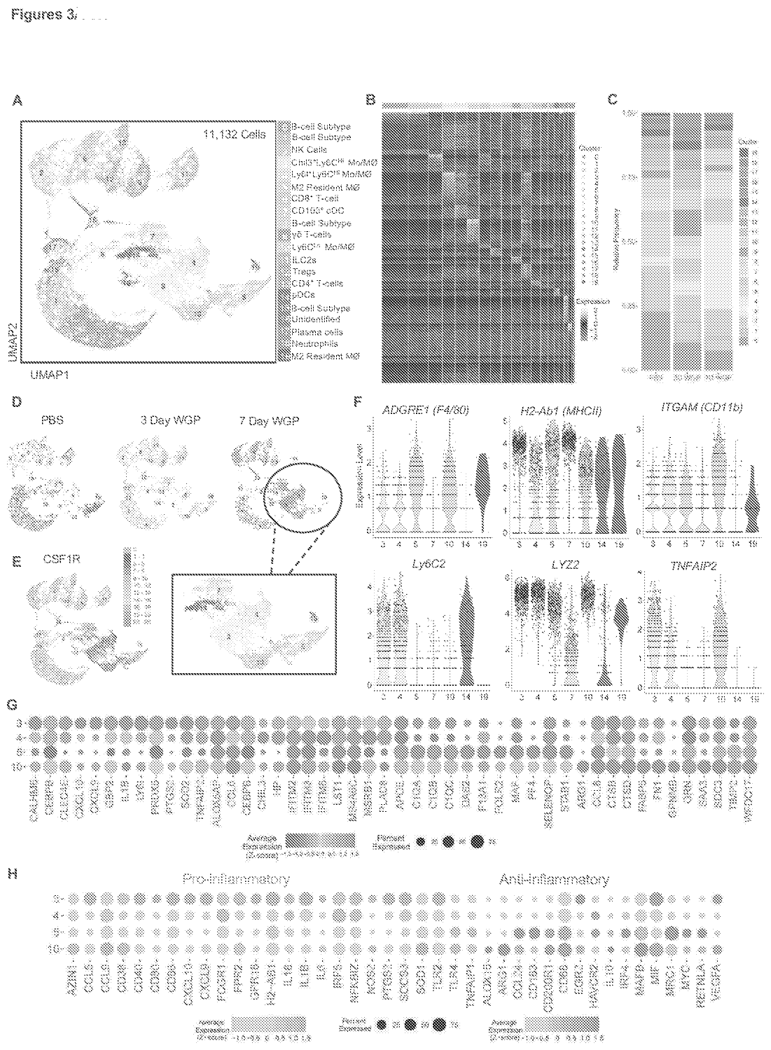

As our CyTOF analyses have revealed the appearance of several new populations to the pancreas due to WGP treatment, to gain a more in-depth understanding of the cell populations present in the pancreas a single-cell RNA sequencing (scRNA-Seq) was performed on sorted CD45⁺ cells from PBS-treated mice and mice treated with WGP three (3-day WGP) and seven days prior (7-day WGP). Two dimensional UMAP representation of 11,132 cells aggregated from three samples with clusters resulting from k-nearest neighbors and Louvain algorithms partitioned into 19 distinct clusters (FIGS. 3A, B). The relative frequency of each cluster within each experimental group was assessed. Here, significant increases in the frequency of clusters 3, 4, and 10 were observed by day 7 after WGP treatment, along with a near disappearance of cluster 5 (FIG. 3C). The relative frequencies of several other populations were also shown to decrease over time. However, this apparent trend is likely due to the relative increase in frequency of other populations. Overall, clusters 3, 4, 10 and 5 appear most significantly altered due to WGP treatment (FIG. 3D).

The populations noted previously to be most significantly altered over time due to WGP treatment were part of the myeloid compartment, as identified by (SFIR expression (FIGS. 3D+3E). As these data along with previous findings suggest that myeloid populations drive the immune changes associated with WGP treatment, the dynamic (SFIR⁺ clusters 3, 4, 10 and 5 were investigated in more detail as shown by violin plots (FIG. 3F) and dot plots representing the top 12 marker genes by average Z-score for each cluster (FIG. 3G). Cluster 5, which was present in PBS mice but virtually disappeared after WGP treatment was identified as resident macrophages. This cluster also notably expressed MAF and APO which are both related to M2 macrophage polarization. Cluster 19 was largely unchanged over time so was not described in more detail, but exhibited an expression profile similar to cluster 5, and thus also represents a subset of tissue resident macrophages. Cluster 10, the Ly6Clo macrophage population, also had a similar expression profile to resident macrophage cluster 5, and notable expression signatures of ARG1 and FABP. We hypothesize this population to be re-polarized resident macrophages given the lack of Ly6C expression, similarity to the resident macrophage population, and the disappearance of the resident population following WGP treatment. Clusters 3 and 4 shared general phenotypic characterization as Ly6CHi infiltrating monocytes/macrophages. Cluster 4 had notable expression of Chil3 and Plac8, which together have been identified by another group to identify Ly6Chi infiltrating macrophages in the kidney. Finally, cluster 3, which was absent in naive mice and showed the greatest increase in relative frequency among all clusters following WGP treatment expressed substantial signatures of TNFAIP2, ILIB, SOD2, and PRIX5, indicating a strongly proinflammatory phenotype. Cluster 3 was thus identified as Ly6CHi inflammatory infiltrating monocytes/macrophages. Interestingly, within the myeloid subset, the populations shown to increase due to WGP treatment are the only populations to express TNFAIP2, indicating that the cells entering the pancreas are likely trained myeloid cells.

To further characterize the activation status of each of the four dynamic myeloid clusters, dot plots were constructed to show the relative expression of genes associated with a pro-inflammatory status and an anti-inflammatory status (FIG. 3H). Clusters 3 and 4, the infiltrating monocytes/macrophages, were shown to be pro-inflammatory, the resident macrophages were anti-inflammatory, and the macrophages in cluster 10 showed an intermediate phenotype. In agreement with previous CyTOF and flow cytometry data, this scRNA-Seq data further characterizes the newly identified myeloid cells in the pancreas to be a heterogenous population of transformed and repolarized Ly6CLo resident macrophages and pro-inflammatory Ly6Chi infiltrating monocyte-derived macrophages that express signatures of trained immunity.

### The WGP-Driven Influx and Training of Myeloid Cells in the Pancreas is CCR2-Dependent and Occurs as Early as 24 Hours Post WGP Treatment

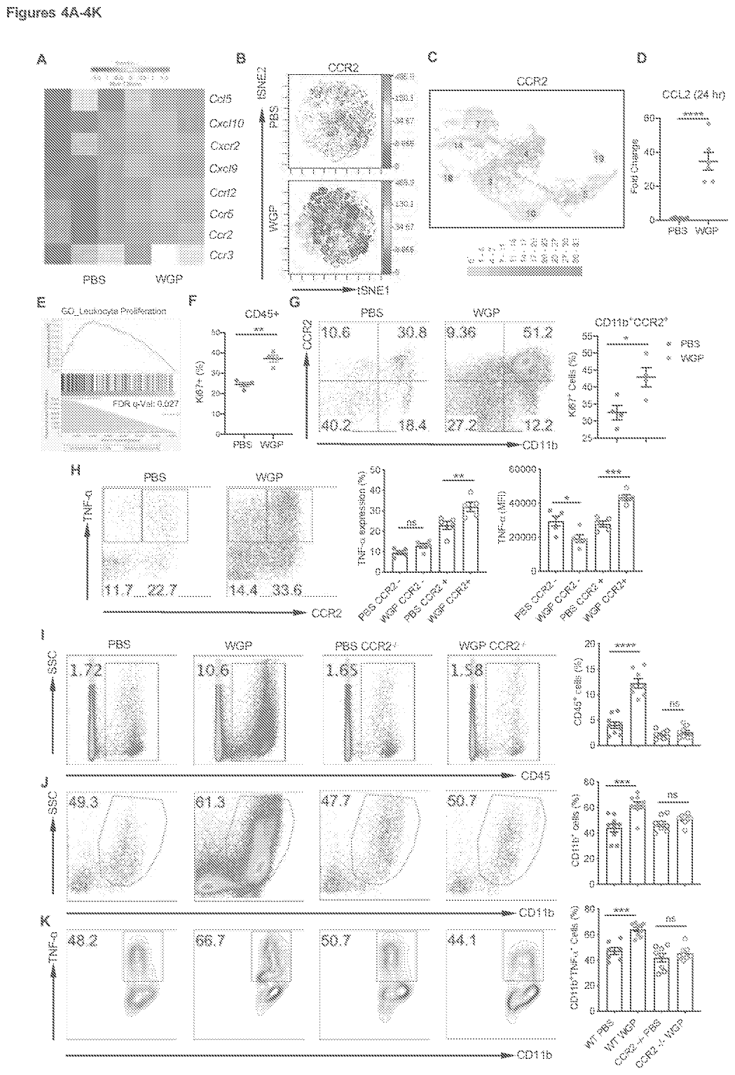

We next examined what mechanisms were responsible for this influx of cells into the pancreas. RNA-Seq data was used to characterize chemokines and cytokines whose expression was significantly upregulated upon WGP treatment (FIG. 4A). While several chemokines and cytokines were upregulated, our observation of macrophage and monocyte influx into the pancreas piqued a specific interest in CCR2 due to its involvement in monocyte and macrophage recruitment and in monocyte egress from the bone marrow. CyTOF data also showed a prominent increase in CCR2 positive cells after WGP treatment (FIG. 4B) and scRNA-Seq showed a distinct expression of CCR2 in clusters 3 and 4, which were the two populations that showed the most distinct phenotypes of trained immunity (FIG. 4C). Additionally, 24 hours post-WGP treatment, whole pancreatic lysates showed a 30-fold increase in CCL2, which is the ligand for CCR2 and is involved in mediating monocyte chemotaxis (FIG. 4D).

RNA-Seq data had shown a clear signature of immune cell recruitment and trafficking, and these data had also indicated that WGP upregulated proliferation of leukocytes and mononuclear cells (FIG. 4E). To investigate whether the CCR2⁺ myeloid cells were proliferating once they reached the pancreas, the percent of CCR2⁺ cells expressing Ki67 was assessed in PBS and WGP treated mice. Following WGP treatment there was an increase in overall proliferating cells (FIG. 4F) and a large percent of these proliferating cells were CD11b⁺CCR2⁺ (FIG. 4G). We then investigated the contribution of CCR2⁺ cells to the trained phenotype. 7 days after in vivo treatment with PBS or WGP, CCR2⁺ and CCR2⁺ populations were measured for a trained response (FIG. 4H). This data indicated that the majority of cells trained following WGP treatment were CCR2⁺. To further examine the role of CCR2 in β-glucan trained monocytes/macrophages, CCR2⁺-mice were trained with WGP β-glucan. CCR2^{-/-} mice did not undergo an influx of CD45⁺ (FIG. 4I) or CD45⁺CD11b⁺ myeloid cells (FIG. 4J) into the pancreas and did not show a trained response as revealed by TNF-α production (FIG. 4K). This data indicates that CCR2 plays a critical role in the migration of innate immune cells to the pancreas and in the induction of peripheral trained immunity in the pancreas.

As previously noted, CCR2 is important for early recruitment of monocytes, but less so for late recruitment. Thus far, we had also observed that at 7 days following WGP training, CCR2 was critical to the recruitment of trained myeloid cells to the pancreas. We next examined whether this influx occurred at an early time point upon WGP training and whether the CCR2⁺ myeloid cells were recruited to the pancreas. To this end, mice were treated with PBS or WGP and pancreatic tissues were analyzed 24 and 48 hours later. Surprisingly, as early as 24 hours after WGP training, increases in CD45⁺, CD11b⁺, F4/80⁺, Ly6C⁺ (FIG. S4D) and CD11b⁺CCR2⁺ cells were observed. Additionally, it was shown that these myeloid cells were trained as early as 24 hours following WGP treatment. This observation highlights a divergence between these results and previously observed phenotypes of trained immunity which usually are not initiated until at least 3 days following training.

### WGP-Trained Pancreatic Infiltrating Myeloid Cells Elicit Potent Trained Responses to Factors Secreted From Pancreatic Cancers and Exhibit Enhanced Phagocytosis and ROS-Mediated Cytotoxicity

As we have shown that using WGP as an initial stimulus results in innate immune cells that are trained to respond more robustly to a secondary exposure of LPS, and that these cells accumulate in the pancreas following treatment, we wanted to know whether secondary stimuli related to pancreatic tumors may also elicit this trained response. To this end, we first investigated whether pancreatic cancer cells themselves are capable of eliciting the WGP-induced trained response. To probe this question, peritoneal macrophages were cultured with PBS or WGP in vitro and 7 days later were restimulated with LPS, the supernatant from cells cultured from a naïve mouse pancreas, and the supernatant from cultured KPC cells, which are a cell line of a pancreatic tumor on a C57BL/6 background derived from the LSL-KrasG12D/⁺; LSL-Trp53R172H/⁺; Pdx1-Cre (KPC) or Pan02 pancreatic cancer cells for 24 hours. TNF-α production in the supernatant was measured by ELISA. Compared to the supernatant from cultured normal pancreatic cells which did not activate previously trained macrophages, the supernatant from cultured KPC and Pan02 pancreatic cancer cells stimulated more TNF-α production in β-glucan trained macrophages. To control for the possibility that the process of phagocytosis itself may cause cells to become activated, peritoneal macrophages were cultured with 3 μm polystyrene microparticle beads, and were then restimulated with PBS or LPS. Results showed that enhance production of TNFα was specific to WGP training.

This was then tested in an ex vivo setting in which mice were treated with PBS or WGP and 7 days later pancreatic myeloid cells were restimulated with the supernatants from cultured KPC or Pan02 cells. It showed that WGP in vivo trained CD11b⁺ myeloid cells in the pancreas produced significantly more TNF-α in response to tumor-conditioned media. Tumor cells themselves secrete a multitude of factors that may specifically function as the second stimulus in trained immunity. It is known that pancreatic tumor cells express high levels of damage associated molecular patterns (DAMPs) and pro-inflammatory factors, such as macrophage migration inhibitory factor (MIF). MIF is a cytokine that is known to be secreted in high concentrations by pancreatic tumors that can act directly on myeloid cells. Indeed, MIF was present in the supernatant of KPC and Pan02 cells as assessed by ELISA. We thus hypothesized that MIF might be a potential tumor-secreted factor that has the capacity to act as a second signal in the setting of WGP-induced trained immunity. To investigate this, pancreatic myeloid cells from in vivo WGP-trained mice were restimulated with a similar concentration of recombinant MIF (rMIF) as present in tumor conditioned media. Pancreatic myeloid cells previously trained with WGP showed enhanced TNF-α production upon rMIF restimulation. Collectively, these data suggest the novel concept that pancreatic tumor cells, through soluble factors that they release, can serve as the second signal to activate myeloid cells in the pancreas that have been trained by WGP.

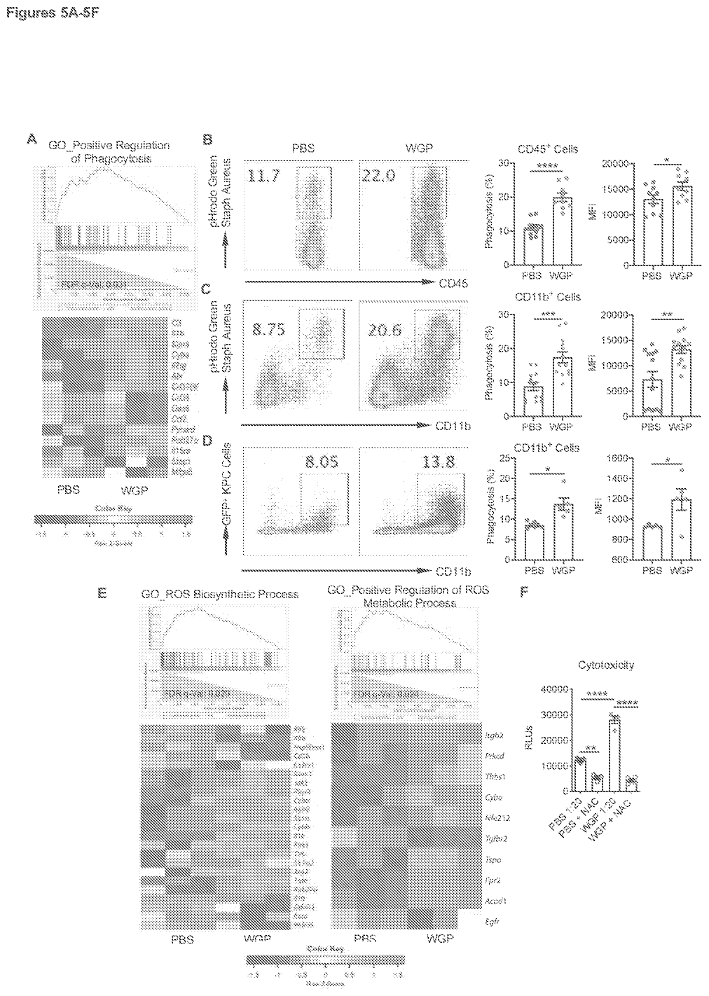

We next examined whether these WGP-trained innate immune cells have enhanced intrinsic anti-tumor properties. RNA-Sequencing data indicated that phagocytosis-related mechanisms were upregulated in the WGP setting, which we hypothesized could be one mechanism of antitumor functionality. CD45⁺ pan immune cells and CD11b⁺ myeloid cells from WGP-trained mouse pancreas were harvested and assayed for phagocytotic potential. WGP treatment led to a significant increase in the phagocytic potential of overall CD45⁺ immune cells (FIG. 5B) and in CD11b⁺ myeloid cells (FIG. 5C). In addition, in vivo trained myeloid cells showed an increase in the phagocytosis of KPC tumor cells (FIG. 5D). We then assessed whether myeloid cells trained by WGP show an increased cytotoxicity to KPC cells. RNA-Seq data indicated that DEGs related to reactive oxygen species (ROS) biosynthetic processes and positive regulation of ROS metabolic processes were significantly enriched in WGP-treated myeloid cells (FIG. 5E). As a result, we hypothesized that the upregulation of ROS production by WGP would result in increased pancreatic myeloid cell cytotoxicity to KPC tumor cells. To explore this hypothesis, CD11b⁺ cells from the pancreas were isolated from β-glucan trained or untrained mice. CD11b+ cells were plated with luciferase expressing KPC cells (KPCLuc⁺) for 24 hours. WGP-trained myeloid cells showed a 3-fold increased cytotoxicity towards KPC tumor cells, and the inhibition of ROS production using the ROS inhibitor N-Acetyl Cysteine (NAC) completely abrogated the WGP-elicited increase in cytotoxicity (FIG. 5F). Ultimately, this data identifies that pancreatic tumor cells are capable of reactivating WGP-trained infiltrating myeloid cells in the pancreas, and that these cells show enhanced phagocytosis and ROS-mediated cytotoxicity.

### WGP-Induced Trained Immunity Reduces Tumor Growth and Prolongs Survival in Orthotopic Models of Pancreatic Cancer

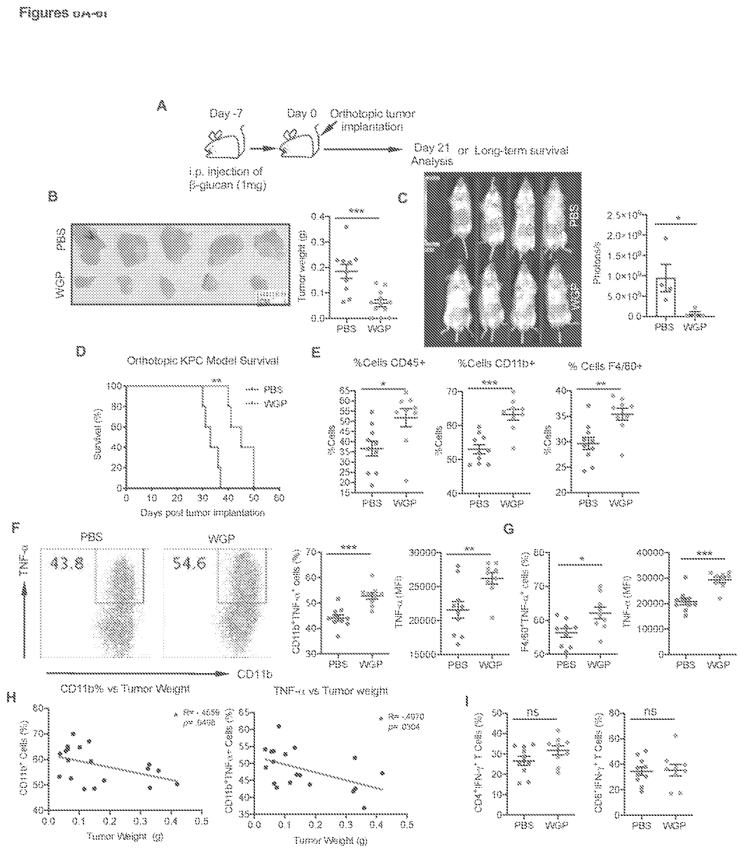

We next investigated whether β-glucan-mediated trained innate immune responses in the pancreas would result in the establishment of an anti-tumor microenvironment that may be sufficient to overcome the characteristically immunosuppressive TME of PDAC. Accordingly, mice were given 1 dose of either PBS or WGP on day −7 and on day 0, 1×10⁵ KPC or KPCLuc⁺ cells were orthotopically implanted into the tail of the pancreas (FIG. 6A). At day 21 mice were euthanized and the tumor weight was measured or (FIG. 6B). In the setting of injection of KPCLuc⁺ cells, mice were injected with luciferase substrate and tumors were imaged (FIG. 6C). Both studies showed a remarkable reduction in tumor burden as a result of WGP treatment, and survival was also significantly prolonged in mice trained with WGP (FIG. 6D).

Immunophenotyping of the tumors showed a persistent increase in CD45⁺ immune cells, CD11b⁺ myeloid cells and F4/80⁺ macrophages in the WGP-trained setting (FIG. 6E). CD11b⁺ myeloid cells (FIG. 6F) and CD11b⁺F4/80⁺ macrophages (FIG. 6G) within the tumor also showed a significant increase in TNFα production due to WGP. Both an increased number of CD11b⁺ myeloid cells (FIG. 6H, left) and an increase in the percent of CD11b⁺ cells producing TNF-α (FIG. 6H, right) significantly correlated with decreased tumor burden. Neither CD4⁺ nor CD8⁺ T-cells showed increased IFN-g, further supporting that the reduction in tumor burden was driven by the WGP-trained myeloid cells (FIG. 6I). Similar antitumor effects were also observed in mice orthotopically implanted with Pan02 tumors. To further confirm that innate immune cells are responsible for the observed anti-tumor immune responses, orthotopic KPC tumors were implanted into NSG mice. Similar to WT mice, NSG mice also showed a significant reduction in tumor size due to WGP training, confirming that the anti-tumor effects of WGP were driven by innate immune cells and functioned independently of adaptive responses. Kalafati et al had shown that innate immune training of granulopoiesis promotes anti-tumor immunity. While we had not seen an important contribution of granulocytes to our phenotype of trained immunity, we examined whether granulocytes are involved in WGP-dependent reduction in pancreatic tumors by depleting neutrophils and observing tumor growth in PBS and WGP treated mice. The depletion efficiency of neutrophils in the pancreas along with the pancreatic tumor burden were assessed. Our results showed a significant reduction in tumor size in the WGP group in the absence of neutrophils.

### Trained CCR2+ Myeloid Cells Are a Primary Effector Cell in the Antitumor Mechanism

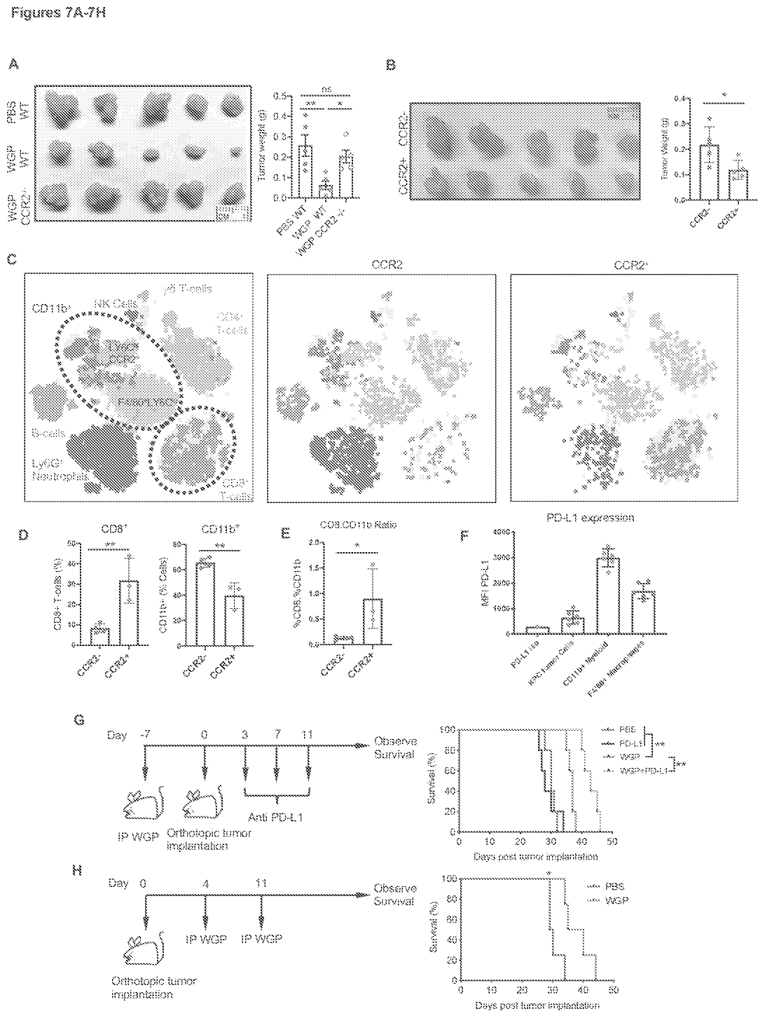

Given that a complete inhibition of innate immune cell trafficking into the pancreas and training of pancreatic myeloid cells following WGP treatment in CCR2^{-/-} mice, we reasoned that CCR2^{-/-} mice would also not show the beneficial anti-tumor immune effects of WGP training. In line with this hypothesis, CCR2⁺ mice that received WGP did not show a reduced tumor burden (FIG. 7A) as compared to WT mice. This demonstrated that CCR2 is requisite for the WGP-driven influx of trained innate immune cells into the pancreas and that those are consequential for the anti-tumor effects. Though CCL2-CCR2 signaling had been identified to be critical in the recruitment of trained monocyte-derived macrophages to the pancreas, we also wanted to know whether the presence of trained HSCs in the bone marrow and the generation of central trained immunity alone was sufficient to slow the growth of orthotopic pancreatic tumors. A BM chimeric mouse model was used where BM cells from PBS or WGP-trained SJL (CD45.1) mice were transplanted into lethally irradiated (950 cGy) congenic B6 (CD45.2) mice. After reconstitution, recipient mice were implanted with orthotopic KPC tumors and tumor size was assessed 14 days later. We observed that there was no tumor size significance between the two groups.

To further confirm the direct anti-tumor functionality of the infiltrating CCR2⁺ myeloid cells, the CCR2⁺ and CCR2⁻ myeloid populations from WGP trained mice were sorted, admixed with KPC tumor cells and implanted orthotopically into mice. Tumors admixed with CCR2⁺ cells were smaller than those that were admixed with CCR2⁻ cells, further supporting that the trained CCR2⁺ myelid cells themselves are a primary effector cell in the antitumor mechanism (FIG. 7B). CyTOF analysis of these tumors revealed that the CCR2⁺ admixed tumors had significantly fewer CD11b⁺ myeloid cells and significantly increased CD8⁺ T-cells present within the tumor (FIGS. 7C+D). The ratio of CD8⁺ T-cells: CD11b⁺ myeloid cells was also significantly increased in the CCR2⁺ admix condition (FIG. 7E).

WGP Synergizes with Anti-PD-L1 mAb Therapy to Prolong Survival in Models of PDAC

Although a significant reduction in tumor burden was shown due to WGP training, it is the case that all mice developed fatal tumors. Our studies had identified that WGP treatment drastically impacted the phenotype of the myeloid populations in the pancreas. Though we had identified that the primary effector cell responsible for the anti-tumor effects of WGP are CCR2⁺ infiltrating monocytes/macrophages, we reasoned that these immune changes may also impact the overall TME in a way that could make adaptive immune cells more responsive to checkpoint blockade therapy. Specifically, because we had observed an increase in the proportion of CD8⁺ T-cells present within CCR2⁺ admixed tumors (FIG. 7C) and had also observed significant PD-L1 expression on myeloid cells present within KPC tumors (FIG. 7F), we hypothesized that WGP treatment may potentiate the effects of anti-PD-L1 mAb therapy.

To assess whether anti-PD-L1 mAb therapy synergizes with WGP-induced trained immunity in the pancreas, PBS or WGP treated mice were implanted with orthotopic KCP tumors were then given either anti PD-L1 mAb or rat IgG2b isotype control mAb. As has been shown in several clinical trials, anti-PD-L1 therapy alone failed to prolong survival even beyond that of the IgG2b isotype control mAb treated mice (FIG. 7B). WGP-trained mice did survive significantly longer than IgG2b isotype and anti-PD-LItreated mice. However, combination of WGP and anti-PD-L1 together prolonged survival most effectively. This shows that there is a clinical benefit to combining WGP with anti PD-L1 immuno-checkpoint blockade therapy.

Thus far, the use of WGP has been described in a setting in which WGP is administered before tumor cells are implanted. Considering the reduction in tumor size of this model, we also tested a more clinically relevant model in which mice were implanted with orthotopic KPC tumors and WGP was used to incite trained immunity thereafter (FIG. 7C). In the therapeutic setting, the WGP22 driven influx of trained myeloid cells to the pancreas was also shown to prolong survival. Together, these data suggest that the initiation of trained immunity in the pancreas using WGP has relevant clinical applications in treating pancreatic cancer that necessitate further translational research and investigation.

### Discussion

While the trafficking of β-glucan has been previously characterized, the specific tropism of β-glucan to the pancreas has not been previously reported. We show that particulate β-glucan can traffic directly into the pancreas and can also be phagocytosed by macrophages which then traffic into the pancreas. While the relationship between the peritoneal cavity and the pancreas has not been well defined, studies relating to the pathophysiology of acute pancreatitis (AP) have identified that peritoneal macrophages are a principal contributor to the inflammatory response in AP, thus supporting a connection between the peritoneum and the pancreas. Our imaging data indeed show that peritoneal macrophages that phagocytose isotope-labeled WGP primarily traffic to the pancreas. Studies on liver injury have further supported this model of a dynamic interchange of cells between the peritoneal cavity and solid organs that is independent of the circulation. Together this suggests that even in homeostatic conditions there exists a basal level of immune cell exchange between the pancreas and peritoneal cavity that can be exploited in the setting of particulate β-glucan.

β-glucan trafficking to the pancreas has a multifactorial impact on the immune populations present within the pancreas. First, β-glucan arrival to the pancreas directly impacts the populations of immunosuppressive M2 resident macrophages present within the pancreas that are known to be important in the promotion of pancreatic tumors. CyTOF and scRNA-Seq data showed a nearly complete disappearance of the resident macrophage population 7 days following WGP administration. Interestingly, the disappearance of the resident macrophage population coincides directly with a reciprocal appearance of a Ly6Clo macrophage population. This Ly6Clo macrophage population bears similar phenotypic markers as the resident population, though skews more towards an MI phenotype. It is thus likely that resident macrophages come into contact with β-glucan that has trafficked to the pancreas and these cells become repolarized, therefore taking on a different cellular phenotype which results in the formation of a unique cluster. Second, the arrival of β-glucan to the pancreas results in amplified chemokine/chemokine receptor signaling which recruits pro-inflammatory Ly6CHi infiltrating monocyte-derived macrophages from the periphery to the pancreas. 24 hours following the administration of WGP, CCL2 levels in whole pancreatic lysates are found to be increased by 30-fold. Accordingly, we show that the robust β-glucan dependent cellular influx to the pancreas is dependent on CCR2.

Although the initiation of an influx of pro-inflammatory immune cells to the pancreas is in itself important, it is that the CCR2⁺ Ly6CHi infiltrating monocyte-derived macrophage populations from the periphery display features of trained immunity which carries the most important implications as this is the first description of the induction of peripheral trained immunity in the pancreas. While CCR2 is known to be an important receptor in the recruitment of monocytes, this is also the first indication that CCR2 signaling on monocytes is requisite for the establishment of peripheral trained immunity. It is noted that CCR2⁺ monocytes/macrophages have been linked to tumor metastasis and progression. However, our data suggest that the recruitment of reprogrammed, trained CCR2⁺ monocytes/macrophages exert potent antitumor effects. This is directly demonstrated by an admix experiment where mice that received CCR2⁺ myeloid cells from WGP-trained mice showed a significantly reduced tumor burden as compared to mice that received CCR2⁻ myeloid cells from the same trained mouse.

As we show that peripheral trained immunity has been established in the pancreas, we ask whether these cells could be re-activated by tumor cells or their secreted factors. Re-exposure of in vivo WGP-trained pancreatic myeloid cells to tumor conditioned media was shown to vigorously elicit a trained response. Others recently showed a role for granulocytes in trained immunity driven anti-tumor mechanisms, however given their use of subcutaneous models of cancer, the currently reported study is the first instance suggesting that myeloid cells in a specific organ can be trained to react directly to tumor cells of that same organ. WGP training also upregulates the direct anti-tumor functionalities of enhanced phagocytosis of tumor cells and ROS mediated cytotoxicity to tumor cells. Further, while we highlight that tumor-conditioned media can reactivate trained myeloid cells, we also identify a specific factor, MIF, in the tumor-conditioned media that is involved in this activation. These data suggest that the induction of trained immunity could participate in mechanisms of tumor immunosurveillance. For example, in early stages of tumor development, tumors secrete soluble factors that can re-stimulate trained innate cells leading to eradication of these tumor cells.

We then translate this understanding of the anti-tumor potential of the trained innate myeloid cells that enter the pancreas to orthotopic models of PDAC, where we observe a dramatic decrease in the tumor burden and an increase in the survival of mice given only one administration of WGP. We further demonstrate that the cells responsible for tumor control are mainly trained CCR2⁺ myeloid-derived cells. However, BM chimeric models suggest that the initiation of central trained immunity within HSCs in the BM and the subsequent induction of systemic trained immunity is not sufficient on its own to slow the growth of pancreatic tumors. This is in contrast to other proposed models of tumor control which appear to be entirely driven by the induction of central trained immunity in the BM. Instead, in this model, the early trafficking of WGP to the pancreas and the resulting production of CCL2 in the pancreas recruits trained CCR2⁺ myeloid cells from the periphery which are mainly responsible for the antitumor effects. WGP directly trafficking to the pancreas also repolarizes M2-like tissue resident macrophages towards M1-like macrophages that may also restrain tumor progression. While the tumor reduction observed in mice implanted with KPC tumors that had been admixed with CCR2⁺ trained cells was significant, it was not as striking as mice given IP WGP. This supports that the antitumor effects of IP WGP are twofold; the recruitment of CCR2⁺ trained anti-tumor myeloid cells to the pancreas and the repolarization of pro-tumorigenic resident macrophages work in concert to elicit antitumor innate responses that lead to reduced tumor burden in models of PDAC.

We also note high expression of MHC II on these trained monocyte/macrophages, which likely plays a role in tumor antigen processing and communication with T-cells to elicit adaptive immune responses against the tumor. Interestingly, while we confirmed that these antitumor mechanisms do not depend on adaptive immune responses, we did observe a significant increase in CD8⁺ T-cells present within CCR2⁺ admixed tumors. Given that myeloid cells in the tumors were observed to display high levels of PD-L1 and that the presence of trained CCR2⁺ cells in the pancreas appeared to recruit CD8⁺ T-cells, we reasoned that WGP may potentiate the effects of anti-PD-L1 therapy. In accordance with the literature, anti-PD-L1 mAb therapy alone had no survival benefit while the combination of WGP and anti-PD-L1 synergized to produce better survival outcomes than WGP alone.

Immunosuppressive myeloid cells play a critical role in the creation of the immunosuppressive TME in PDAC. Here, we demonstrate a novel capability to engage these myeloid cells in the setting of PDAC through the induction of trained innate immunity in the pancreas. Importantly, we also show that the novel ability to initiate trained immunity in the pancreas potentiates the therapeutic effects of immunotherapy such as anti-PD-L1 immuno-checkpoint blockade therapy. Lending further clinical applicability to these findings, we also exhibit that the induction of trained immunity in the adjuvant setting can decrease tumor size and prolong survival. These findings emphasize the potential of using β-glucan to therapeutically target myeloid cells within the TME and highlight that the induction of peripheral trained immunity in the pancreas could play a consequential role in reprogramming the suppressive TME of PDAC which could result in extending the life of patients diagnosed with this deadly malignancy.

### Methods

### Mice

Six to eight week-old female wild-type (WT) C57BL/6J mice were purchased from the Jackson Laboratory (Bar Harbor, ME, USA) or bred in the University of Louisville specific pathogen-free (SPF) animal facility. C57BL/6 Dectin-1 knockout (Dectin-1−/−) mice were described previously. CCR2 global knock-out mice were purchased from Jackson Laboratory. Albino C57BL/6 mice were kindly provided by Dr. Jonathan Warawa at the University of

Louisville. NOD/SCID/IL2rgNull (NSG) mice and B6/SJL-CD45.1 were purchased from the Jackson Laboratory. All mice were at least 6 weeks of age upon use, and all experiments involving animals were performed in compliance with all relevant laws and institutional guidelines provided by the Rodent Rearing Facility (RRF) and approved by the Institutional Animal Care and Use Committee (IACUC) of the University of Louisville.

### Preparation of β-Glucan

Highly purified particulate β-glucan in the form of particulate Whole β-glucan Particles (WGP) isolated from Saccharomyces cerevisiae was provided by Biothera. Before use, WGP was gently sonicated for 15 seconds, 2 times using a Qsonica Q55-110 Q55 Sonicator (Cole-Parmer) to ensure aggregates were broken up.

### Preparation and Use of DTAF WGP

DTAF (Sigma Aldrich) at 2 mg/mL was mixed with a suspension of 20 mg/mL WGP in borate buffer (pH 10.8). This incubated at room temperature for 8 hours with continuous mixing. Following incubation, the WGP was centrifuged and washed with cold sterile endotoxin-free DPBS (Sigma Aldrich) 5 times or until the supernatant no longer contained visible DTAF. The concentration was adjusted to 10 mg/mL in the endotoxin-free DPBS for storage. 1 mg of the DTAF WGP was injected IP into C57BL/6 and Dectin-1^{-/-} mice and organs were harvested 3 days later.

### Preparation of the ⁸⁹Zr-WGP

WGP (100 mg) was mixed with Deferoxamine-SCN (2.7 mg, in 0.8 ml DMSO) and suspended in 10 ml sodium carbonate buffer (0.1 M, pH 9.4) overnight at room temperature in the dark with gentle shaking. The Deferoxamine-labeled WGP was then washed with DI water (10X 40 mL), and 30 mg of Deferoxamine-labeled WGP was mixed with 3 mCi of ⁸⁹Zr oxalate in 2 ml Tris·HCl buffer (0.5 M, pH 7.5), and then incubated at 37° C. for 60 min with shaking. The ⁸⁹Zr-WGP was then centrifuged and washed with 3 ml of sterile PBS. The radioactivity of ⁸⁹Zr-WGP was measured by a dose calibrator and used for in vitro and in vivo studies.

### Biodistribution and PET/CT Scan Using ⁸⁹Zr-WGP

Positron Emission tomography (PET)/computed tomography (CT) imaging was conducted in C57BL/6 and Dectin-1^{-/-} mice 48 hours after IP injection of 1 mg of pure ⁸⁹Zr-WGP or injection of 1×10⁶ peritoneal macrophages that had been co-cultured with 25 μg/ml of ⁸⁹Zr-WGP for 2 hours and then gently washed to remove excess ⁸⁹Zr-WGP. The mice were scanned for 15 min with a Siemens R4 MicroPET and followed by 10 min of CT scan. Siemens IAW software was used for the acquisition and reconstruction of the PET signal, and Siemens IRW software was used for merging and analyzing the imaging data. At the end of the imaging study, mice were euthanized, and organs of interest were harvested. For biodistribution, 50 μL of peripheral blood was collected using a retrobulbar bleeding technique. The brain, heart, lungs, liver, spleen, kidneys, pancreas, large intestine, small intestine, stomach, femur, a piece of skin from the flank of the mice and the rectus femoris muscle were harvested, weighed, and placed in a 2470 Wizard automatic gamma counter (PerkinElmer) in order to measure the radioactivity of each tissue. The CPM values were calculated using Prism software (GraphPad Software, La Jolla, CA).

### Pancreatic Processing

Following euthanization, mouse pancreases were harvested and gently cut into smaller pieces using sterile scissors. They were suspended in a 15 mL tube in complete media (RPMI) with 1X digestion buffer comprised of 300 U/ml collagenase I, 60 U/ml Hyaluronidase, and 80 U/ml DNase (Sigma). These were placed in a rotating incubator at 37° C. with 5% CO₂ for 15-20 minutes. The digestion buffer was then quenched with ice cold complete RPMI 1640 and washed. Cells were passed through a sterile nylon 40 μm basket filter and small undigested pieces of tissue were smashed using a sterile syringe stopper in order to generate a single-cell suspension. If an appreciable number of red blood cells (RBCs) were seen to exist in the sample,

RBC lysis was performed by adding 2 mL of sterile 1033 ACK (Thermo Fisher Scientific).

### In Vivo WGP Administration

Mice were given a single 1 mg intraperitoneal dose of gently sonicated WGP, 3 μm polystyrene beads (Sigma Aldrich), or 3 um fluorescent microspheres (Polysciences) (all 1 mg in 200 μl of sterile PBS) or 200 μl of sterile PBS on day 0. For typical trained immunity studies, 7 days following the initial IP dose, mice were euthanized using CO2 and the pancreas along with other tissues of interest were removed and processed. For dose titration studies, 0.5 mg, 1 mg and 2 mg of WGP were delivered IP in 200 μL of sterile PBS. For time titration studies, mice were injected with 1 mg of WGP and the pancreas was harvested after 24 hours, 48 hours, 3, 7, 10, 16 and 30 days later.

### Ex Vivo Re-Stimulation

In order to assess the trained phenotype of mononuclear cells in mice treated with WGP or PBS ex vivo, after processing the pancreas, pancreatic cell suspensions were plated in 24 well plates and stimulated with LPS (10 ng/ml), the supernatant from cultured KPC and Pan02 cells (40%), and recombinant MIF (rMIF) (10 ng/ml). rMIF was a generous gift from Dr. Robert Mitchell at the University of Louisville and was prokaryotically expressed, purified, and re-folded a described previously. Cells were cultured in DMEM, and incubated at 37° C. with 5% CO₂ for 5-6 hours in the presence of 1X brefeldin A (Biolegend). The cells were then harvested using a cell scrapper, washed, pelleted and then stained for intracellular cytokine expression.

### In Vitro Training and Re-Stimulation Assay

Peritoneal macrophages or sorted CD11b⁺ cells from a mouse pancreas were plated in a 24 well plate for 2 hours at 37° C. and 5% CO₂ to allow for the attachment of cells to the plates, after which the floating cells were gently aspirated. Attached cells were gently washed with sterile PBS and then resuspended in 1 mL of DMEM constituted of 10% FBS 1% penicillin/streptomycin. For the initial training of cells, 25 μg/ml of particulate WGP, 25 μg/ml of 3 μm polystyrene microparticle beads (Sigma-Aldrich) or 100 μL of PBS were added to the appropriate well and incubated for 24 hours. After 24 hours, wells were gently washed to remove the initial stimulus and fresh DMEM was added and cells were incubated with 5% CO₂at 37° C. for 7 days. After 7 days, the media was aspirated, replaced with fresh media, and cells were re-stimulated with LPS (100 ng/ml), the supernatant of KPC or Pan02 cells (40%), or PBS as a control. 24 hours after stimulation, the supernatants were harvested and used in an ELISA for TNF-α and IL-6.

### Tumor Conditioned Media

1×10⁶ KPC or Pan02 cells were cultured in a six well plate in 4 mL of complete DMEM and at 37° C. and 5% CO₂. After 3 days the supernatants were harvested and stored at −80° C. in aliquots for use as tumor-conditioned medium. As a control, the pancreas of a C57BL/6 mouse was processed into a single cell suspension and 1×10⁶ of these cells were cultured in a six well plate in 4 mL of complete DMEM and at 37° C. and 5% CO₂, and supernatants were also collected after 3 days. As a control, the pancreas of naïve mice were processes into a single cell suspension and plated in a 6 well plate for 1 day. Non-adherent cells were washed away and these cells were then cultured for 3 days and the supernatant was harvested.

### Acquisition of Peritoneal Macrophages

Mice were euthanized and 5 mL of sterile RPMI was injected into the peritoneum using a 25-gauge syringe. The abdomen was massaged to liberate the peritoneal macrophages. A small incision was made and a transfer pipette was used to remove the suspension. The peritoneal cavity was then washed several times with cold RPMI and cells were pelleted at 1600 RPM.

### Flow Cytometry

Single-cell suspensions in PBS with 1% FBS were blocked with murine Fc Block (anti-CD16/CD32) at 4° C. for 15 minutes. Fluorochrome labeled antibodies for viability (APC/Cy7) and to surface markers CD45, CD11b, F4/80, Ly6G, Ly6C, MHCII, CD3,CD4, CD8, NK1.1,Ki67 (Biolegend) and CCR2 (R&D Systems) were used. After 30 minutes of incubating in the dark at 4° C., cells were washed with cold PBS, and filtered through a 40 μm mesh filter. The samples were acquired using a FACSCanto II cytometer (BD Biosciences) and analyzed using FlowJo software (Tree Star, Ashland, OR).

### Intracellular Staining for Expression of Cytokines

Following stimulation, cells were stained for the desired surface markers as described above. The cells were then washed with cold PBS, and 500 μl of fixation buffer (Biolegend) was added to the tubes, briefly vortexed and incubated in the dark at room temperature for 20 minutes. 1 ml of Permeabilization buffer (Biolegend) was then added and samples were centrifuged at 1600 rpm for 5 minutes at 4° C. followed by one more wash using 1 ml of permeabilization buffer. Cells were resuspended in 200 μl of permeabilization buffer and cells were stained with antibodies against TNF-α, Ki67, Granzyme-B, IL-12, IL-6 and IFNg or the respective isotype control overnight at 4° C. Cells were then washed, filtered and data was acquired using a flow cytometer.

### FACS Isolation of CD11b⁺ Macrophages for In Vitro Training

The pancreas was processed into a single cell suspension as described above. Cells were washed with 1 mL of PBS, incubated with Fc block for 10 minutes at 4° C. followed by staining with viability dye (APC-Cy7), CD45 (PerCPcy5) and CD11b⁺ (APC) for 30 minutes at 4° C. Cells were washed with PBS and re-suspended in cold MACS Running Buffer (Miltenyi Biotech). Viability-CD45⁺CD11b⁺ cells were sorted using a FACS Aria III (Bd Biosciences). Cells were collected in a 50% FBS,40% PBS, 10% HEPES (Corning). After sorting the cells were washed with PBS and then plated for in-vitro training, as described above.

### ELISA

Supernatants from in vitro trained cells along with standards were analyzed using murine TNF-α and IL-6 ELISA kits (BioLegend). The assay was performed per the manufacturer's instructions and all conditions were performed in triplicates. An ELISA kit for r-MIF (R and D systems) was also used according to the manufacturer's instructions in order to quantify r-MIF in Pan02 and KPC tumor conditioned media and the supernatant from cultured untreated pancreatic cells.

qRT-PCR

After CD11b⁺ cells had been isolated from the mouse pancreas in WGP treated and untreated mice, cells were saved in TRIzol. RNAs were isolated and reverse transcribed using the TaqManReverse Transcription Reagents (qRT-PCR) amplification using the BioRad MyiQ single color RT-PCR detection system. Briefly, cDNA was amplified in a 25 μL reaction mixture consisting of SYBR Green PCR super-mix (BioRad), 100 ng of complementary DNA template, and selected primers (200 nM) using the recommended cycling conditions.

### H+E

7 days following injection with PBS/microparticle beads or WGP, the pancreata were harvested and fixed in 4% formalin for 1 week followed by embedding in paraffin according to standard procedures. Paraffin embedded tissues were cut into 5 mm thick sections and strained with hematoxylin and eosin (H & E) for morphological analysis.

### Serum Amylase Measurement

Murine serum amylase was measured using the Amylase Activity Assay Kit (Millipore Sigma) and was used according to the manufacturer's instructions. In short, mice were injected with WGP and 7 days later, blood was collected from mice using a retro-bulbar bleeding technique. These samples were used to assay for serum amylase.

### In Vivo T-Cell Depletion

T-cells were depleted using an anti-CD4 mAb alone, anti-CD8 mAb alone or anti-CD4 and anti-CD8 mAbs together. Antibodies were made in-house. In this depletion procedure, mice were injected IP with WGP on day 1 and were also injected IP with 200 μg of the mAbs at day 1 and day 4 during the treatment period. Mice were euthanized on day 7. (CD-4 clone GK1.5, CD-8 clone 53-6.72). Depletion efficiency was confirmed on day 7.

### In Vivo NK Cell Depletion

NK cells were depleted through the intraperitoneal injection of 100 μg of PK136 mAb (Produced in the laboratory of Dr. Jun Yan at the University of Louisville) on days −1 and 5 during the treatment period. WGP was injected at day 0, and on day 7 animals were euthanized and the depletion efficiency of NK cells was assessed by staining pancreatic tissues for NK1.1.

### Ly6G Depletion

Neutrophils were depleted by injecting 300 μg of anti-Ly6G mAb (Bio X Cell) or isotype control Rat IgG2a (Bio X Cell) at day-1, 2 and 6 during the course of treatment. 1 mg of WGP was injected IP on day 1. Mice were euthanized on day 7 and the pancreas was assessed for efficiency of depletion. In the tumor model mice were injected with 300 μg of anti-Ly6G mAb or isotype control Rat IgG2a on day-2, 4, 10 and 16. Mice were given WGP at day 0, and were implanted with orthotopic KPC pancreatic tumors on day 7. Mice were euthanized on day 21 and pancreatic tissues were stained with Ly6G to assess granulocyte depletion efficiency at that time. CyTOF mass cytometry sample preparation. Mass cytometry antibodies were either purchased from Fluidigm or were created in house by conjugating commercially available purified antibodies to the appropriate metal isotope using the MaxPar X8 Polymer or MCP9 Polymer kits (Fluidigm). Pancreatic samples from three PBS and three 7-day WGP mice were processed into a single cell solution and ex-vivo stimulation was performed as described above. Cells were gently scraped from the plates using a sterile cell scraper, washed with PBS and placed into a sterile culture tube. 2×10⁶ cells per samples were used. Cells were first stained for viability with 5 μM cisplatin (Fluidigm) in serum free RPMI1640 for 5 minutes at RT. Cells were then washed with RPMI1640 containing 10% FBS for 5 minutes at 300×g. Cells were stained with the surface antibodies for 30 minutes at RT and washed twice with Maxpar Cell staining buffer (Fluidigm). For staining on intracellular cytokines, cells were then fixed with 1 mL of 1X Maxpar Fix I buffer for 30 minutes at RT and then washed twice with 2 mL of 1X Maxpar Perm-S buffer for 5 minutes at 800×g. The cytoplasmic/secreted antibody cocktail was then added and incubated with the cells for 30 minutes at RT. Following incubation, cells were washed with 1 mL of 1X Maxpar Perm-S buffer for 5 minutes at 800×g and gently blotted to remove all liquid from the tube. In order to stain for nuclear antigens cells were then suspended in 1 mL of 1X Maxpar nuclear antigen staining buffer for 30 minutes at RT. The nuclear antigen antibody cocktail was then added and incubated for 30 minutes at RT. Cells were washed with twice for 5 minutes at 800×g with 2 mL of Nuclear Antigen Staining Permeability buffer. Finally, cells were fixed with 1.6% formaldehyde for 10 minutes at RT, then incubated overnight in 125 nM of Intercalator-Iridium (Fluidigm) at 4° C.

### CyTOF Data Acquisition

Once cells were ready for acquisition, samples were washed twice with Cell Staining Buffer (Fluidigm) and kept on ice while awaiting acquisition. Directly prior to acquisition, cells were suspended in a 1:9 solution of Cell Acquisition Solution: EQ 4 element calibration beads (Fluidigm). A Helios CyTOF system was used, and following proper startup and tuning procedures, samples were run at a rate of less than or equal to 500 events/second up to 300,000 events. Using the CyTOF software, .FCS files were normalized into .fcs files, and these files were then ready to for data analysis.

### CyTOF Data Analysis

CyTOF data was analyzed using FlowJo, the CytoBank software package 44, and the CyTOF workflow 45 which includes a suite of packages available in R (r-project.org). For analysis conducted within the CyTOF workflow, FlowJo workspace files exported from flow Workspace and CytoML were used.

### RNA Sequencing: RNA Extraction and Isolation

7 days following administration of PBS or WGP IP, pancreata from were harvested, processed into a single cell suspension as described previously, stained for viability, CD45 and CD11b as described previously, and sorted using a FACS Aria III as described previously. Samples were prepared in triplicate for each experimental group. Once these myeloid cells were isolated, cells were washed 2× with ice cold PBS and then lysed with Trizol (Invitrogen). RNAs were extracted using a QIAGEN RNAeasy Kit (QIAGEN). The isolated RNA was checked for integrity using the Agilent Bioanalyzer 2100 system (Agilent Technologies, Santa Clara, CA) and quantified using a Qubit fluorometric assay (Thermo Fisher Scientific, Waltham, MA). Poly-A enriched mRNASeq libraries were prepared following the Universal Plus mRNA-Seq kit standard protocol (Tecan Genomics, Redwood City, CA) using 10 ng of total RNA. All samples were ligated with Illumina adapters and individually barcoded. Absence of adapter dimers and consistent library size of approx. 300 bp was confirmed using the Agilent Bioanalyzer 2100. The library concentration and sequencing behavior was assessed in relation to a standardized spike-in of PhIX using a Nano MiSeq sequencing flow cell from Illumina. 1.8 pM of the pooled libraries with 1% PhiX spike-in was loaded on one NextSeq 500/550 75 cycle High Output Kit v2 sequencing flow cell and sequenced on the Illumina NextSeq 500 sequencer targeting 60M 1×75 bp reads per sample.

### RNA Sequencing

Libraries were prepared using the Universal Plus mRNA-seq kit with with NuQuant® library quantification (NuGen). The six samples were spread across four sequencing lines in one run. The 24 single-end raw sequencing files (.fastq) 50 were downloaded from Illumina's BaseSpace 51 (https://basespace.illumina.com/) onto the KBRIN server for analysis. Quality control (QC) of the raw sequence data was performed using FastQC (version 0.10.1). The sequences were aligned to the mm 10 mouse reference genome using STAR (version 2.6), generating alignment files in bam format. Differential expression of ENSEMBL protein-coding transcripts was performed using DESeq2. Raw counts were obtained from the STAR aligned bam format files using HTSeq (version 0.10.0). The raw counts were normalized using the Relative Log Expression (RLE) method and then filtered to exclude genes with fewer than 10 counts across the samples.

### RNA Sequencing: Gene Set Enrichment Analysis

Gene set enrichment analysis (GSEA) was used to further characterize the biology of the genes comprising the WGP vs PBS conditions and their differences. Gene sets were obtained from the Molecular Signatures Database (MSigDB) for Gene Ontology (GO) biological processes and reactome pathways. For each gene set, all tested gene locations in the comparison of WGP vs PBS treated control mice were sorted from highest to lowest significance using p values. This approach allows highly significant up- and down-regulated genes to be included within each gene set, an approach that more accurately reflects the conditions in a biological pathway. For this analysis, a table of the enriched sets is followed by an enrichment plot displaying the profile of the enrichment score (ES) score and position of gene set members on the rank ordered list.

### Single-Cell Sequencing: Isolation of Single Cells and RNA Sequencing

Live CD45⁺ cells were sorted from mouse pancreata, washed and resuspended in 1× PBS (calcium and magnesium free) containing 0.04% BSA. Single cells were captured and barcoded cDNA libraries were constructed using the Chromium Next GEM Single Cell 3′ Reagent Kit (v3.1, 10X Genomics) and the Chromium Controller, according to manufacturer's instructions. Libraries were pooled and sequenced using a 28 bp×8 bp×125 bp configuration for read1×i7 index×read2 on the Illumina NextSeq 500 with the NextSeq 500/550 150 cycle High Output Kit v2.5 (20024907).

### Single-Cell Sequencing: Gene Expression Profiling

Bcl files were demultiplexed into fastq files using the CellRanger software (10X Genomics, v3.1.0). The total number of sequenced reads was 506,913,062. The reads were of good quality as determined by FastQC. Gene counts were measured using CellRanger ‘count’, utilizing the cell ranger-mm 10-3.0.0 reference genome for mouse. A counts matrix was generated for each individual sample and one aggregated sample with the expected number of cells set at 5,000.

The raw count data determined by CellRanger was used as input to a custom analysis pipeline in R which uses a variety of single-cell analysis tools based on Seurat. The knee plot displays a graph showing the ranked UMI counts for each cell barcode for data aggregated across the three groups. Cells above the inflection point represent possible doublets while those below the knee represent background cells. Cell quality control measures were analyzed using Seurat v3, and cell barcodes with the following characteristics were removed from the analysis: low counts (possible background cells) with an FDR cutoff of 0.01 from the DropletUtils function ‘empty Drops’, high counts (possible doublet cells) with more counts than the knee plot inflection point, mitochondrial content greater than 30% and ribosomal content greater than 40%. Gene (features) were further filtered to remove retired gene identifiers, and genes that were not expressed in at least two cells.

The expression data was normalized using SCTransform where cell cycle genes, ribosomal content, and mitochondrial content were regressed. The cells were then clustered and dimension reduction was performed using UMAP. Initial cluster names were assigned using a modified mGSVA enrichment score technique. For each of these clusters, the top marker genes were identified. Differentially expressed genes comparing each cluster to every other cluster (all pairwise comparisons) was determined using Seurat and MAST.

Identification of Clusters Generated With scRNA-Seq

Non-myeloid-derived clusters were classified generally as β-cells (MS4A1), plasma cells (SD) (1), CD8⁺ T-cells (CD3e, C8a), CD4⁺ T-cells (CD3e, CD4), T-regulatory cells (T-regs) (CD3e, CD4, FoxP3), gd T-cells (CD3e, TRGC1, TRGC2, IL7Ra) and, type 2 innate immune cells (ILC2s) (Alox5, KLRG1, Ly6a, Pparg, GATA3, IL-5, IL-13, and Rxrg). Neutrophils were identified through MMP9, Csf3r, S100A8, S100A8 and ADAM8 expression, though may be underrepresented in these analyses due to their low RNA content and high levels of intrinsic RNases. Conventional dendritic cells (cDCs) were identified through ITGAX and ITGAE expression and plasmacytoid DCs (pDCs) were identified by ITGAX and Siglech.

Classification of Myeloid Clusters from scRNA-Seq

Cluster 5 expressed ITGAM^{IntADGRE}1^{Hi}Lyz2^{Hi}H2-Ab1^{Hi}Ly6C2⁻ and did not express TNFAIP2, indicating that these cells are resident macrophages. Cluster 10 expressed ITGAM^{Hi}ADGRE1^{Hi}Lyz2^{Hi}H2-Ab1^{Int}Ly6C2⁻. Cluster 3 and 4 expressed ITGAM^{Int}ADGRE1^{Int} Lyz2^{Hi} H2-Ab1^{Int} Ly6C2^{Hi}, which suggests that both are subsets of infiltrating monocytes/macrophages, though the enhanced inflammatory genes expressed in cluster 3 were used to identify cluster 3 as an inflammatory infiltrating monocyte/macrophage.

### Phagocytosis Assays

The pancreas of PBS/microparticle injected mice and in-vivo WGP-trained mice were harvested 7 days after injection and processed as described previously into a single cell suspension. 2×10⁶ were washed with HEPES dilutes 50× in RPMI 1640 and then incubated in 100 μL of this solution for 1 hour at 37° C. in order to activate the cells. The Invitrogen pHrodo™ Green S. aureus BioParticles™ Phagocytosis Kit for Flow cytometry (Thermo Fisher Scientific) was used according to the manufacturer's instructions. 100 μL of the reconstituted particles or 1×10⁶ GFP⁺ KPC tumor cells were added to the activated pancreatic cells and incubated for 1 hour at 37° C. Samples were gently vortexed every 15 minutes. The reaction was stopped by added 1 mL of coldPBS. Samples were incubated with Fc Block for 10 minutes at 4° C., stained for viability, CD45, CD11b and F4/80 for 30 minutes at 4° C., and then analyzed using a BD FACSCanto. For analysis, after gating on live cells and CD45, cells that fluoresced in the FITC channel were determined to be phagocytic.

### Cytotoxicity Assay

The pancreas from WGP and PBS treated mice were harvested and the CD11b⁺ populations were isolated using magnetic CD11b⁺ MicroBeads (Miltenyi Biotec) and an autoMACS Pro Separator (Miltenyi Biotec). Purified CD11b⁺ cells were then washed and counted, and these were plated at a ratio of 1:20 tumor:effector cells in a 96 well plate. All experimental samples were run in triplicate. After plating the CD11b⁺ cells, the ROS inhibitor N-acetyl-1-cysteine (NAC) (Sigma Aldrich) was added to one set of PBS and WGP derived cells at 1 mM for 1 hour before the addition of 5000 luciferase expressing KPC⁺ pancreatic tumor cells to all wells. After 24 hours, of coculture, the plates were centrifuged and 20 μL of the supernatant was mixed with 100 μL of the Luciferase Assay Reagent (Promega). Luciferase activity measured in the supernatant correlated with tumor cells that had been killed by the effector cells and was measured using a luminometer (Femtomaster FB 12, Zylux Corporation). The spontaneous luciferase signal from plated tumor cells was subtracted from the measurement of the supernatant. Luciferase values are represented as Relative Light Units (RLUs).

### In Vivo Tumor Models of Pancreatic Cancer

A KPC cell line on a C57BL/6 background derived from the LSL-KrasG12D/⁺; LSL⁻ Trp53R172H/⁺; Pdx1-Cre (KPC) mouse model was purchased from Ximbio. These and Pan02 cells which were a generous gift from Dr. Yong Lu at Wake Forest University, were used in an orthotopic model of pancreatic cancer. A KPC line transfected with GFP and luciferase (KPCGFP⁺Luc⁺) were also a generous contribution from Dr. Michael Dwinell at the Medical College of Wisconsin. These cells were used exclusively in albino C57BL/6 mice. For tumor implantation, mice were anesthetized using isoflurane, and the abdomen of the mice were pepped with betadine and draped in a sterile fashion. A 2 cm midline laparotomy was performed using aseptic technique with sterile instruments. Following laparotomy, the pancreas and spleen were externalized. Tumor cells were suspended in ice cold PBS and mixed in a 1:1 ratio with basement membrane matrix Matrigel (Corning). 0.1×10⁶ tumor cells in 50 μL of the PBS-matrigel solution were injected into the tail of the pancreas using a 30-guage insulin syringe. The formation of a small bubble indicated successful implantation. The peritoneum was closed using coated polyglycolic acid braided absorbable 5/0 suture and the skin was closed using silk braided nonabsorbable 5/0 suture. (CP Medical). Buprenorphine was administered for pain management up to 72 hours following surgery and mice were monitored.

### In Vivo Imaging

Mice implanted orthotopically with GFP⁺ Luciferase⁺ KPC tumor cells were injected IP with 150 mg/kg of body weight at 100 μL of XenoLight D-Luciferin-K⁺ Salt Bioluminescent Substrate (Perkin Elmer). After 10 minutes, mice were anesthetized with isoflurane and placed inside of a Biospace Lab Photon Imager, which is a dedicated low light level in vivo optical modality for bioluminescent and fluorescent imaging. Images of mice were taken and used to measure tumor size and growth.

### Admixture Tumor Model

Mice were trained with 1 mg of WGP and 7 days later the CD11b⁺CCR2⁺ and CD11b⁺CCR2⁻ populations were sorted and mixed 1:1 with KPC tumor cells 1×10⁵ tumor cells. Cells were implanted orthotopically into WT mice and 3 weeks later mice were euthanized, and tumor size was assessed.

### Generation of BM Chimeras

To generate BM chimera, B6/SJL (CD45.1) mice were treated with IP PBS or WGP and 7 days later the BM was harvested. WT mice (CD45.2) were lethally irradiated (950 cGy) and 2×10⁶ CD45⁺ BM cells from the B6/SJL PBS or WGP treated mice were transplanted IV. Six weeks later, the peripheral blood of recipients was analyzed to check the success of the BM transplant, and mice were implanted with 0.1×10⁵ KPC cells orthotopically. 14 days later mice were euthanized and tumor size was assessed.

Combination Therapy with Anti PD-L1 mAb

C57BL/6 mice were treated with WGP at day-7 and implanted with KPC orthotopic pancreatic tumors on day 0. Mice were treated with 200 μg of anti-mouse anti-PD-L1 mAbBio X Cell) or Rat IgG2b isotype control (Bio X Cell) at day 3, 7 and 11. Mice were then monitored for survival.

### WGP as a Treatment

C57BL/6 mice were implanted with KPC orthotopic pancreatic tumors at day 0 and on day 4 and 11 mice were given 1 mg of IP WGP or PBS. Mice were then monitored for survival.

### Statistical Analysis

Results are represented as mean±SEM. Data were analyzed using a two-tailed Student's t test or Mann-Whitney U-test. Multiple-group comparisons were performed using a one-way or two-way ANOVA followed by Tukey's multiple comparisons test. Correlation analyses were performed using Pearson correlation coefficient (normal distribution). Statistical significance was set at p<0.05. All statistical analyses were performed using GraphPad Prism Software Version 9 (GraphPad Inc., La Jolla, CA).

### EXAMPLE 2

### Induction of Trained Immunity Controls Cancer Metastasis through the Metabolic Sphingolipids-Mitochondrial Fission Pathway

Metastasis is the leading cause of cancer-related deaths and innate immune cells play a critical role in the metastatic microenvironment. Trained immunity induces an increased responsiveness of innate immune cells to subsequent heterologous challenges. Here, we explore the implications of harnessing the pre-metastatic niche myeloid cells through induction of trained immunity using a natural compound yeast-derived whole β-glucan particle (WGP). WGP in vitro training in macrophages results in an increased responsiveness to not only the conventional secondary stimulus LPS but also to tumor cells and tumor-derived factors. WGP in vivo treatment leads to a trained phenotype in lung interstitial macrophages (IMs) as a consequence of emergency myelopoiesis in the bone marrow. Induction of trained immunity by WGP inhibits tumor metastasis and prolongs tumor-free survival in multiple mouse models of tumor metastasis. Lung IMs trained with WGP exhibit enhanced phagocytic capacity and cytotoxicity against tumor cells in a ROS-dependent manner. Further studies reveal that WGP-induced trained immunity in lung IMs is mediated by a metabolite sphingosine-1-phosphate (S1P) through the sphingolipid synthesis pathway. S1P induces the phosphorylation of dynamin-related protein-1 (Drp-1) which is followed by mitochondrial fission to elicit a trained response with enhanced mitochondrial ROS production and subsequent killing of tumor cells. Inhibition of mitochondrial fission abrogates WGP-induced trained immunity both in vitro and in vivo and subsequent inhibition of lung metastases. These results identify a novel sphingolipid synthesis-S1P-mediated mitochondrial fission pathway for WGP-induced trained immunity and cancer metastasis control.

### Introduction

The advent of immunotherapy has revolutionized cancer treatment and has led to a rapid decline in cancer-related deaths. Despite improved clinical efficacy with immune checkpoint blockade therapy, the recurrence and metastasis of cancer remains a major challenge in the clinic. Metastasis accounts for more than 90% of cancer-related deaths and is a major cause of cancer-related morbidity and mortality in humans. Accumulating evidence suggests that even prior to tumor metastasis, extracellular factors released from the primary tumors into the circulation are able to prime the distant secondary organs to generate a favorable environment that is supportive for tumor survival and growth. This phenomenon has been termed as a pre-metastatic niche, which prepares a receptive soil for incoming fertile tumor cells. Tumor-derived factors have been extensively studied and are known to modulate immune cells, including innate myeloid cells in the target organs to make them amenable to support tumor growth. In fact, the innate myeloid cell signatures and pathways are among the most significantly enhanced features within the pre-metastatic microenvironment. Therefore, harnessing innate myeloid cells towards an antitumor phenotype may provide immune surveillance to eliminate or control tumor metastasis.

Innate immune cells are conventionally not believed to retain a memory phenotype, which is a hallmark of adaptive T and B lymphocytes. However, there is accumulating evidence suggesting that invertebrates, lower vertebrates, and plants are able to respond adaptively to recurrent infections despite lacking the memory features afforded by the adaptive immune system. These observations have led to the development of a novel concept of innate immune memory, also known as trained innate immunity or trained immunity. Long-term reprogramming of innate immune cells occurs upon exposure to an exogenous insult through metabolic, epigenetic, and transcriptomic changes which result in an increased responsiveness to a non-specific secondary insult. The concept of trained immunity has been explored for its application in health and disease, especially infectious diseases and inflammatory conditions. However, the role of trained immunity in the context of cancer, particularly for the control of cancer metastasis, is yet to be fully explored.

Many biological agents, including polysaccharide fungal β-glucans, have the ability to induce trained immunity. β-Glucan is a natural compound existed in the cell walls of a variety of micro-organisms and plants including mushrooms, yeasts, oats, barley, seaweeds, algae and bacteria. β-Glucans are active polymers of D-glucose units linked together by glycosidic bonds that vary in their glycosidic linkages, branches, lengths, three-dimensional conformation and solubility depending on the source of β-glucans. β-Glucans therefore vary in their physical and chemical properties contributing to the differences in their functional activities. Fungal β-glucans from Candida albicans and Trametes versicolor have been well-studied for their ability to induce trained immunity. β-Glucans derived from S. cerevisiae has also been widely used as an immunomodulatory agent in cancer. These β-glucans have particulate and soluble forms. Particulate β-glucan, also known as whole glucan particles (WGP), has been used as a nutraceutical supplement. We hypothesize that natural compound particulate β-glucan WGP can stimulate trained immunity and induction of trained immunity could be used to effectively control cancer metastasis through modulation of myeloid cells within the pre-metastatic niche.

In this study, we showed that particulate β-glucan WGP induces a potent trained innate immune response. WGP-trained macrophages not only respond to LPS as a secondary stimulus, but also elicit a trained response upon exposure to tumors cells and tumor-derived soluble factors. Further studies revealed that lung interstitial macrophages (IMs) are trained by WGP in vivo treatment, leading to inhibition of cancer metastasis to the lungs. WGP training also significantly increases phagocytosis and cytotoxicity of lung IMs against tumor cells in a reactive oxygen species (ROS)-dependent manner. Furthermore, a novel metabolic sphingolipid synthesis pathway-mediated mitochondrial fission is demonstrated as an underlying mechanism for WGP-mediated trained immunity and cancer metastasis inhibition.

### Results

### Yeast-Derived Particulate β-Glucan WGP Induces Trained Immunity in Macrophages

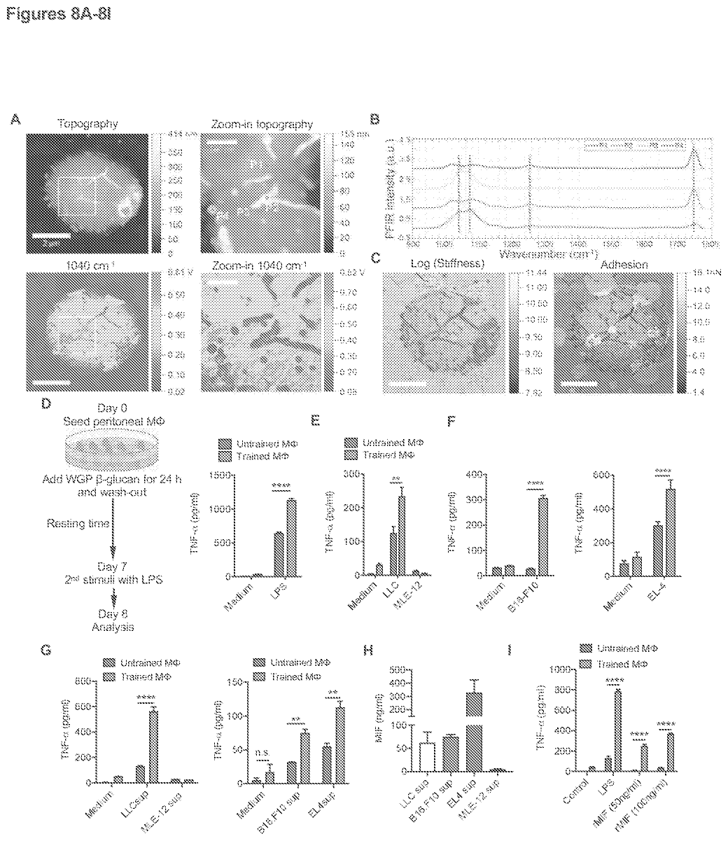

Before using WGP to induce trained immunity, we first characterized this form of β-glucan by combining peak force infrared (PFIR) microscopy with super-resolution fluorescence microscopy, which enabled simultaneous chemical, topographical, and mechanical mapping of WGP with ˜6 nm resolution (FIGS. 8A-8C). We found that the WGP is mainly composed of β-glucan, as indicated by the IR peaks near 1140-1160 cm⁻¹, but also displays some heterogeneous surface features (P2 and P4 in FIG. 8A) that are enriched in lipid-containing components, as indicated by the intense IR peak at ˜1750 cm⁻¹ which is characteristic of lipid ester groups. Using GFP tagged Dectin-1 RAW264.7 cells, we observed that WGP was phagocytosed by RAW cells and dectin-1 was then recruited and clustered at the site of the phagocytic cup, suggesting that dectin-1 is the critical receptor for WGP phagocytosis by macrophages. We next determined whether WGP was able to induce trained immunity. A well-established in vitro training experiment was performed (FIG. 8D). WGP-trained peritoneal macrophages showed an enhanced TNF-α response to LPS re-stimulation as compared to untrained macrophages (FIG. 8D), indicating that WGP induces trained immunity. Peritoneal macrophages were also treated with polystyrene beads that were the same size as WGP and no enhanced TNF-α response was observed compared to untreated cells after LPS re-stimulation. These data suggest that the induced trained immunity is WGP specific.

To address whether WGP-trained macrophages could elicit a trained response against tumors, we used tumor cells as secondary stimuli. WGP-trained peritoneal macrophages induced a stronger TNF-α response when stimulated with Lewis lung carcinoma cells (LLC) as compared to untrained controls. Stimulation of WGP-trained macrophages with mouse lung epithelial cell line, MLE-12, was unable to induce a trained response (FIG. 8E), suggesting that tumor-specific factors stimulate the trained response. This effect was also shown by culturing WGP-trained macrophages with B16F10 melanoma and EL4 lymphoma cells (FIG. 8F), emphasizing that elicitation of trained response is not specific to a single tumor cell type. Tumor cells can secrete a variety of factors to generate a favorable environment for their growth and metastasis. To determine if tumor-secreted factors could act as a secondary stimulus for a trained response, WGP-trained macrophages were re-stimulated with LLC or MLE-12 culture supernatants. Peritoneal macrophages trained with WGP produced a significantly higher TNF-α when re-stimulated with LLC culture supernatant as compared to untrained controls (FIG. 8G). MLE-12 culture supernatant, however, failed to show this effect. In addition, B16F10 and EL4 culture supernatants also stimulated trained responses (FIG. 8G), suggesting that tumor-derived soluble factors can induce a trained response when used as a secondary stimulant. To further explore which tumor secreted factors are responsible for the induction of a trained response, we quantitated one of the most widely studied tumor secreted cytokines, macrophage migration inhibitory factor (MIF). We observed a significant amount of MIF ranging from 50 ng/ml to 350 ng/ml in the tumor culture supernatants (FIG. 8H). When WGP-trained peritoneal macrophages were re-stimulated with physiologically relevant concentrations of recombinant MIF protein (rMIF), a dose-dependent enhanced TNF-α response was observed (FIG. 8I); this indicates that tumor-secreted MIF can serve as a secondary stimulus to induce a trained response. Previous studies have shown that both tumor cells and macrophages are capable of releasing MIF. To confirm that it is the tumor-secreted MIF responsible for the trained response, peritoneal macrophages from MIF knockout (KO) mice were used in in vitro WGP training. We observed that WGP-trained MIF KO macrophages elicited a significantly higher TNF-α response upon stimulation with LPS or LLC culture supernatant compared to untrained controls, similar to WT macrophages. This data indicates that tumor-derived MIF is capable of inducing a trained response. Collectively, these results suggest that WGP is able to induce a potent trained response upon re-exposure to not only LPS but also to tumor-derived factors such as MIF.

### WGP In Vivo Treatment Alters Myeloid Cell Composition in the Lungs as a Consequence of Bone Marrow Myelopoiesis

We next examined whether WGP treatment in vivo could also induce trained immunity using a standard in vivo training protocol. In this protocol, wildtype (WT) C57BI/6 mice received an intraperitoneal (IP) administration of WGP. Emergency myelopoiesis is an important attribute of β-glucan-induced trained immunity. β-Glucan modulates hematopoietic progenitors and induces expansion of myeloid progenitors in the bone marrow (BM). We examined whether WGP has a similar effect. BM cells harvested from in vivo WGP treated mice showed an increased percentage of Lin⁻Sca-1⁺c-Kit⁺ (LSK) hematopoietic progenitor/stem cells (HPSC) and CD48⁺CD150⁻ LSK cells also known as multi-potent progenitors (MPPs). To understand if the observed BM myelopoiesis is a direct effect of WGP, we labelled WGP with fluorescent dye DTAF and injected into mice. BM cells were harvested after 48 h and a small percent of DTAF-labeled WGP was seen in the BM, but not in the lungs. To attest whether WGP-induced BM myelopoiesis results in systemic increase in myeloid cells and subsequent trained response, spleen and inguinal lymph nodes were harvested from PBS vs WGP-trained mice. Both spleen and inguinal lymph nodes showed an increase in CD11b⁺ myeloid cells. In addition, a trained response was observed as revealed by an increased TNF-α expression on F4/80+ macrophages.

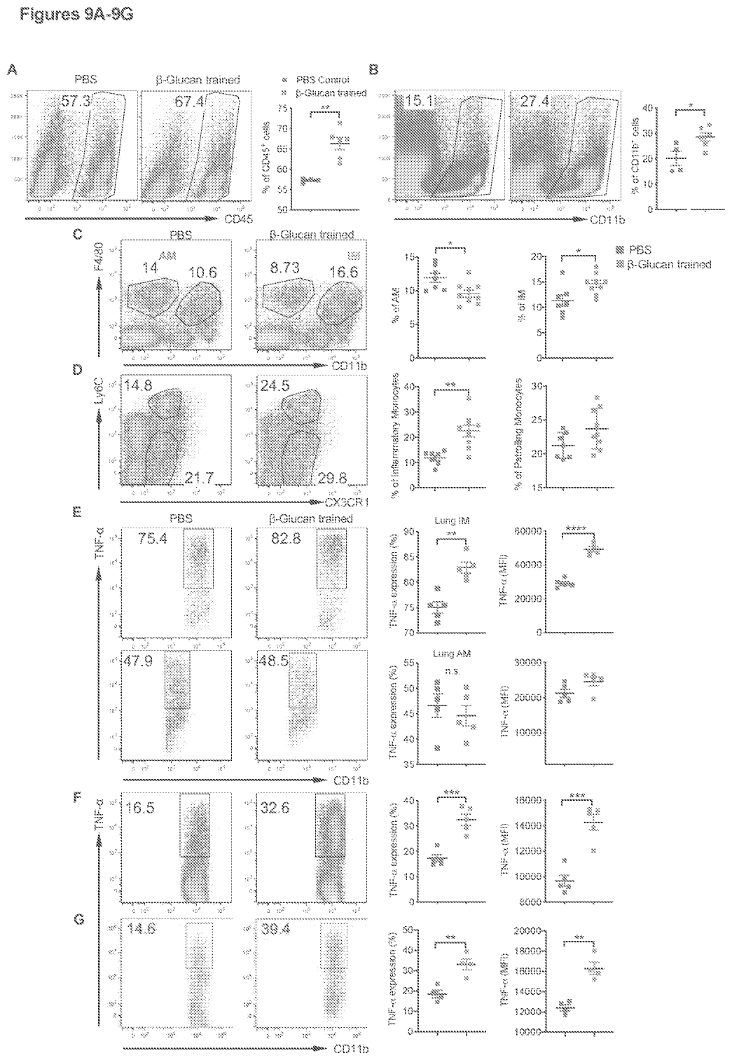

We next sought to determine whether WGP could induce similar changes in the lung myeloid cell compartment as the lung is the common site of tumor metastasis. Mice treated with WGP had significantly increased CD45⁺ immune cells (FIG. 9A) and CD11b⁺ myeloid cells (FIG. 9B) in the lungs compared to PBS controls. Further phenotyping of the myeloid cell population showed an increase in CD11b^{+F}4/80⁺ IMs (FIG. 9C) and a decrease in CD11b⁻F4/80⁺ alveolar macrophages (AMs) (FIG. 9C) in WGP-treated mice compared to the controls. We also observed a significant increase in the frequency of CD11b⁺Ly6C^{hi}CX3CR1⁺ inflammatory monocytes and a slight increase in the frequency of CD11b⁺Ly6C^{low}CX3CR 1+ patrolling monocytes after WGP treatment (FIG. 9D). To examine whether WGP treatment induces trained immunity in lung myeloid cells, lung mononuclear cells from mice that had received IP WGP injections for 7 days prior were stimulated ex vivo with LPS and the intracellular TNF-α expression was assayed. Lung IMs from WGP treated mice showed a significantly increased expression of TNF-α both in terms of percentages and mean fluorescence intensity (MFI). Lung AMs, however, did not show any significant difference in TNF-α expression between WGP-treated and-untreated mice (FIG. 9E). To further determine if the dose of WGP that we used for our in vivo training experiments was optimal, we treated mice IP with 0.5 mg or 2 mg of WGP and assayed for TNF-α expression in lung IMs. WGP training showed a dose-dependent increase in TNF-α expression in lung IMs. However, there was no observable differences between 0.5 ng and 2 mg of WGP treatment. Therefore, the dose of 1 mg was used for our subsequent experiments. To ensure that the lung IM trained phenotype was a result of a WGP-induced effect, polystyrene beads that have similar size as WGP were IP injected into mice and 7 days later the lungs were harvested. Lung IM showed a significant increase in TNF-α expression in WGP-trained mice as compared to PBS-treated mice, but not between PBS-treated and polystyrene-bead-treated mice. To corroborate our previous observation of in vitro training assay using tumor cells and tumor derived factors as a secondary stimulus, we stimulated lung mononuclear cells from in vivo WGP-trained or -untrained mice with LLC culture supernatants or rMIF. WGP-trained lung IMs showed a significantly higher TNF-α expression n response to both LLC culture supernatants (FIG. 9F) and rMIF (FIG. 9G) compared to PBS controls. Taken together, these results suggest that WGP in vivo training induces emergency myelopoiesis in the BM, resulting in a systemic increase in myeloid cells. In addition, lung Ims—but not AMs—are increased and trained by WGP in an in vivo setting.

### WGP-Induced Trained Immunity Significantly Reduces Tumor Metastases and Prolongs Tumor-Free Survival

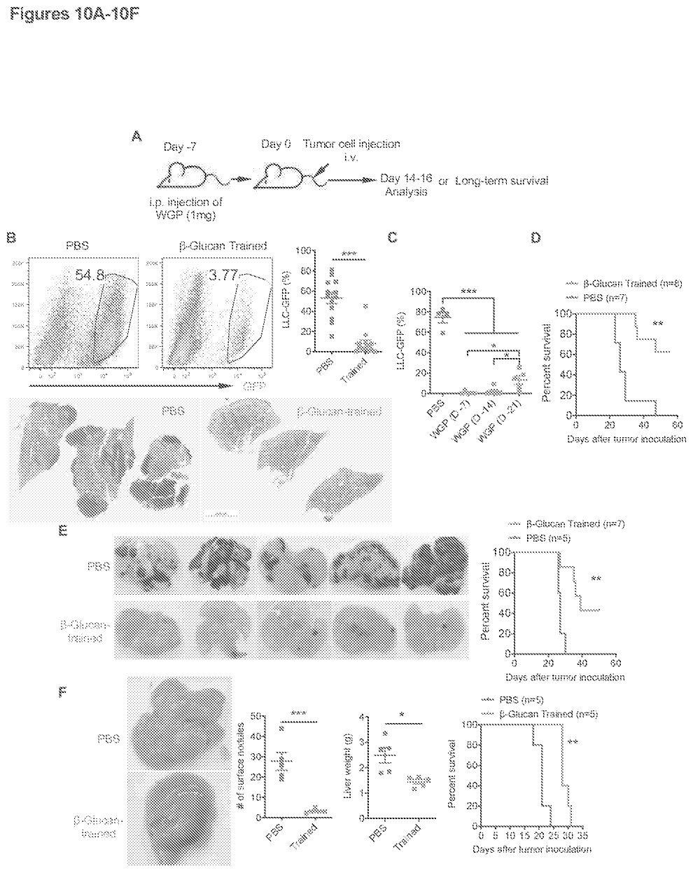

Since WGP treated lung IMs elicit potent trained immune responses against tumor-derived factors, we hypothesized that induction of trained immunity may effectively control tumor metastasis in the lungs. Mice trained with WGP for 7 days were intravenously (i.v.) injected with green fluorescent protein tagged LLC cells (LLC-GFP) and were either euthanized after 14-16 days to determine the tumor burden in the lungs or observed for long-term survival (FIG. 10A). WGP-trained mice were found to have a significantly reduced tumor burden in the lungs as compared to PBS controls. The histopathology analysis of lung sections corroborated the flow cytometry results, showing increased tumor nodules in the control lung sections and few to none in the lung sections from WGP-trained mice (FIG. 10B). Given that short-term memory or short-lived memory is one of the hallmarks of trained innate immunity, we set out to determine how long the WGP-mediated trained effect lasted. Mice were trained with WGP at day −7, day −14 and day −21 followed by LLC-GFP injection on day 0 and euthanized on day 14. WGP-treated mice had significantly reduced tumor burdens compared to untrained PBS control (FIG. 10C). However, there was a marked increase in the tumor burden in mice trained at day −21 compared to the mice treated at day −7 and day −14, suggesting that the WGP-induced trained response is short-lived and may last up to a month. Further analysis of the lungs revealed that WGP-training led to a decrease in the frequency of regulatory T cell (Tregs) and an increase in the frequency of TNF-α expressing CD4⁺ and CD8⁺ T cells. Macrophages from the tumor-bearing lungs, also referred to as metastasis-associated macrophages (MAMs), have potent immunosuppressive function. We found that MAMs from WGP-trained mice had significantly reduced immunosuppressive activity on effector T cells and revealed a higher frequency of IFN-γ⁺CD4⁺ T cells compared to MAMs from PBS controls. These results suggest that WGP-trained macrophages in the lung may promote adaptive anti-tumor T cell responses. Long-term survival studies revealed a significantly prolonged survival in mice trained with WGP compared to PBS controls (FIG. 10D). WGP training resulted in a 60% tumor-free survival until 60 days post tumor challenge.

To validate that WGP-mediated training and metastasis inhibition are not only specific to an LLC metastasis model, a melanoma lung metastasis model was employed. A similar in vivo WGP training protocol was used and mice were injected with B16F10 melanoma cells i.v. and examined for tumor burden in the lungs at day 14-16 or observed for long-term survival. Mice trained with WGP had significantly reduced tumor metastases in the lungs as revealed by fewer black tumor nodules compared to PBS controls (FIG. 10E). In addition, WGP training significantly prolonged the survival of B16F10 challenged mice when compared to the control group. Approximately 50% of mice in the WGP-trained group survived 60 days following tumor injection, whereas all the mice in the control group died by day 30 (FIG. 10E). To assess whether WGP-mediated training and metastasis inhibition are limited to lung metastasis models, we employed a liver metastasis model where mice were injected i.v. with EL4 lymphoma cells. Previous studies have shown that EL4 lymphoma i.v. injection leads to liver metastasis. WGP-trained mice showed a significantly reduced number of tumor nodules (observed as white dots in the liver) that were accompanied by reduced total liver weights (FIG. 10F). WGP training also significantly prolonged the survival of these mice (FIG. 10F, right), emphasizing the systemic benefit of a trained response mediated by WGP treatment.

WGP-induced lung IM trained immunity is critical in cancer lung metastasis control and inhibition of spontaneous lung cancer development

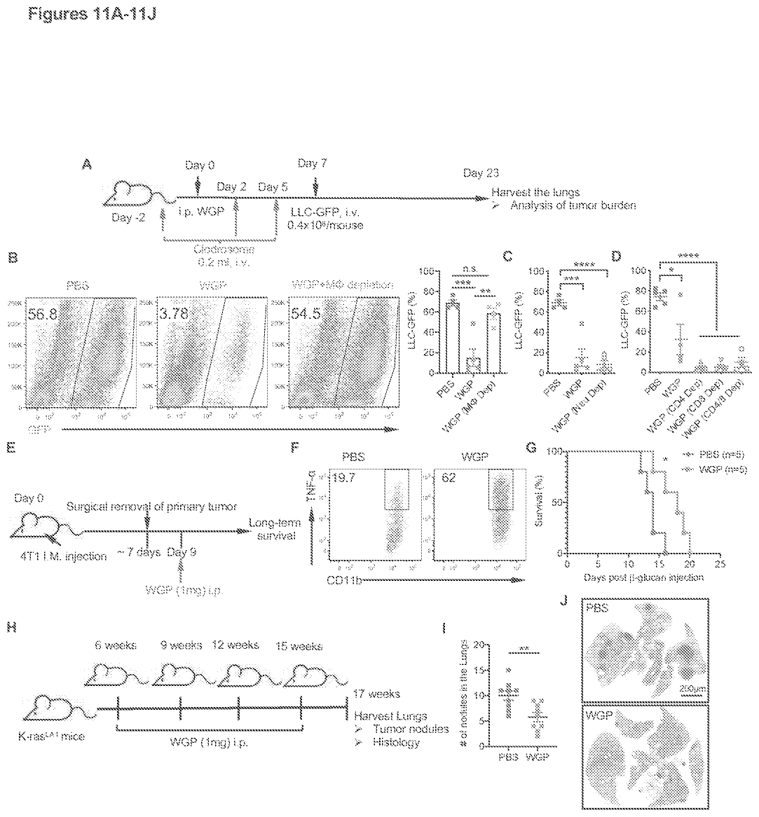

To determine whether WGP-trained IMs are the effector cells responsible for controlling tumor metastasis, we depleted macrophages with i.v. injection of Clodronate liposome (Clodrosome). Previous studies have shown that i.v. injection of Clodrosome leads to approximately a 70% reduction of macrophages in the lungs. Mice were injected with Clodrosome two days before β-glucan treatment and continued Clodrosome injection on days 2 and 5. On day 7, mice were injected i.v. with LLC-GFP (FIG. 11A). Depletion of macrophages resulted in significantly increased tumor burdens comparable to PBS controls (FIG. 11B). These data suggest that macrophages are required for WGP-mediated metastasis inhibition. We also examined whether innate neutrophils are involved in WGP mediated lung cancer metastasis inhibition, as a recent study emphasized the role of trained immunity-mediated granulopoiesis in an anti-tumor phenotype in subcutaneous tumors. We depleted neutrophils prior to and during the WGP training period followed by tumor challenge. No substantial difference was observed in the lung tumor burdens between WGP-trained mice and neutrophil-depleted-WGP-trained mice (FIG. 11C). In addition, depletion of CD4⁺ or CD8⁺ T cells, or both, did not affect WGP-mediated lung tumor metastasis inhibition (FIG. 11D). These results collectively suggest that WGP-mediated training and metastasis control are not dependent on neutrophils and adaptive T cells.

To mirror the clinical setting of lung metastasis for potential clinical translation, we used a clinically relevant model where the triple negative breast cancer cell line 4T1 was orthotopically implanted on the 4^{th} mammary gland of Balb/c female mice. Previous studies have shown that 4T1 mammary tumors can spontaneously develop lung metastases. When tumor size reached 3-4 mm in diameter, primary tumors were surgically excised. Mice were then treated with WGP and long-term survival was monitored (FIG. 11E). Since we used Balb/c mice that are genetically distinct from C57B1/6 mice, we confirmed the in vivo trained phenotype of lung IM in this strain as revealed by enhanced TNF-α expression upon WGP training (FIG. 11F). Mice trained with WGP showed a significantly prolonged survival as compared to the controls (FIG. 11G), indicating thatWGP can be used in an adjuvant setting to inhibit lung metastasis.

To further elaborate on the use of WGP in a therapeutic setting of tumor development, we developed a treatment protocol using a genetically engineered mouse model (GEMM) that spontaneously develops lung cancer. We used a K-ras^{LA1} mutated mouse model that bears a common mutation observed in human lung cancer patients. K-ras^{LA1} mice spontaneously develop tumor nodules in the lungs starting at 4 months of age. A significant number of nodules started appearing at 6 months of age and the lungs were fully covered in tumor nodules beyond 9 months of age. Taking this tumor development and short-lived nature of WGP-mediated trained response into consideration, we developed a treatment protocol where K-ras^{LA1} mice were trained with WGP starting at 6 weeks of age and repeated every 3 weeks at 9, 12 and 15 weeks. At 17 weeks, mice were euthanized to examine tumor nodules in the lungs (FIG. 11H). WGP-treated mice had significantly fewer tumor nodules in the lungs as compared to untreated controls (FIG. 11I). Histological analysis of the lungs also confirmed this phenotype (FIG. 11J). Taken together, these results suggest a therapeutic benefit of WGP-mediated lung IM trained immunity in controlling lung tumor development and metastasis.

### WGP Training Augments Lung IM Phagocytosis and Cytotoxicity

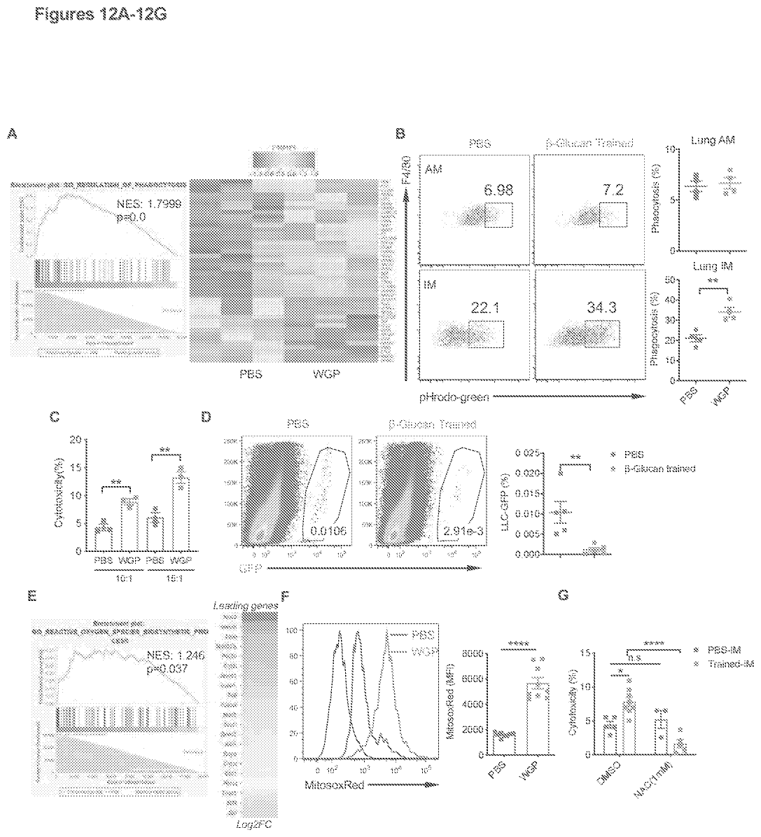

To understand the mechanistic basis of WGP-induced lung IM training and inhibition of tumormetastasis, we performed RNA sequencing (RNAseq) on lung IMs from WGP-trained or control mice. RNAseq analysis revealed a significant number (total 3417) of differentially expressed genes (DEGs) in WGP-trained lung IMs compared to PBS controls. Ingenuity Pathway Analysis (IPA) indicated pathways involved in innate immune function including phagocytosis, cytokine secretion, the NFκB pathway, the oxidative stress and metabolic pathways including lipid metabolism pathways. Gene Set Enrichment Analysis (GSEA) also showed a significant enrichment of innate immune effector functions in IMs from WGP-trained mice. Phagocytosis-related genes were significantly enriched in WGP-trained lungIMs (FIG. 12A). To validate this result, we performed phagocytosis assay using pHrodo-green labelled Staphylococcus aureus with lung AMs or IMs from PBS-vs WGP-trained mice. No significant changes in the phagocytosis were observed in AMs from PBS- and WGP-trained mice. However, lung IMs from WGP-trained mice exhibited significantly increased phagocytosis compared to those from PBS control mice (FIG. 12B), indicating that WGP training increases lung IM phagocytic capacity.

To investigate whether the increased phagocytosis results in an increased cytotoxicity against tumor cells, we performed a cytotoxicity assay by co-culturing sorted lung IMs with different ratios of LLC cells. WGP-trained IM were found to have significantly increased cytotoxicity compared to untrained PBS controls (FIG. 12C). To determine if an increased cytotoxicity was observed in vivo, PBS-vs WGP-trained mice were injected with LLC-GFP cells i.v. and euthanized after 24 h later to analyze the LLC-GFP cells in the lungs. WGP-trained mice showed a significantly reduced frequency of LLC-GFP cells as compared to PBS controls (FIG. 12D), suggesting that WGP-mediated training increases lung IM cytotoxicity, resulting in reduced tumor seeding and engraftment in the lungs.

One of the enriched pathways that we observed from our GSEA analysis was the reactive oxygen species (ROS) biosynthetic pathway (FIG. 12E). To further understand if generation of ROS was an underlying mechanism for cytotoxicity of WGP trained macrophages, we treated macrophages with WGP and detected mitochondrial ROS (mtROS) using MitoSox Red staining. WGP treatment significantly increased mtROS production compared to untreated controls (FIG. 12F). To determine whether the increase in mtROS was responsible for the increased cytotoxicity of WGP-trained lung IMs, a cytotoxicity assay was performed in the presence or absence of ROS inhibitor, N-acetyl-L-cysteine (NAC). Addition of NAC significantly inhibited the cytotoxicity of WGP-trained IMs (FIG. 12G). Taken together, these results suggest that WGP-induced lung IM trained immunity results in an increased phagocytosis and enhanced mtROS production, which together result in increased cytotoxicity to tumor cells. Sphingolipid synthesis, specifically sphingosine-1-phosphate (S1P) is critical for the induction of trained immunity in lung IMs

Activation of mammalian/mechanistic target of rapamycin (mTOR) through a hypoxia-inducible factor-1α (HIF-1α) pathway mediated aerobic glycolysis is a well-established mechanism for inducing trained immunity. To determine if the mTOR and HIF-1α pathways are important for the induction of trained immunity by WGP, we generated myeloid cell conditional knockout mouse (cKO) models for Raptor (mTORC1), Rictor (mTORC2), and HIF-1α. Peritoneal macrophages from Raptor cKO mice trained with WGP showed a significantly increased TNF-α response after LPS re-stimulation compared to the un-trained cells. In addition, WGP-trained control and Raptor cKO mice showed a significantly reduced tumor burden as compared to the untrained mice. No difference was noted between control and Raptor cKO mice. Similar results were shown in Rictor cKO mice (data not shown). These data suggest that WGP-mediated trained immunity and subsequent inhibition of tumor metastasis are not dependent on the mTOR pathway.

To investigate if the HIF-1α pathway is important for WGP-mediated training and metastasis inhibition, we performed a similar in vitro training experiment with sorted peritoneal macrophages from HIF-1α cKO and control mice. Peritoneal macrophages from WGP-trained HIF-1α cKO mice showed a significantly increased TNF-α response compared to untrained controls. However, TNF-α levels from WGP-trained HIF-1α cKO peritoneal macrophages were significantly lower compared to these in control mice, suggesting that the WGP-mediated trained response is partially dependent on the HIF-1α pathway. Despite this, HIF-1α cKO mice were able to significantly reduce lung metastases upon WGP-training and was comparable to WGP-trained control mice. These data suggest that the HIF-1α pathway is not essential in WGP training-mediated metastasis inhibition.

β-Glucan-mediated IL-1β signaling has also been implicated in the proliferation of HPSCs and myelopoiesis. To examine if the WGP-mediated trained response and metastasis control are dependent on the IL-Iβ pathway, we performed both in vitro training and in vivo training-tumor challenge experiment using IL-IR global KO mice. Upon WGP training, peritoneal macrophages from IL-IR KO mice produced a significantly higher TNF-α compared to untrained controls. IL-IR KO mice trained with WGP were also able to significantly inhibit lung metastasis compared to untrained controls, indicating that IL-IR signaling is not critical for WGP-mediated training and metastasis inhibition. Along this line, we performed an in vitro training assay using peritoneal macrophages from Nlrp3 KO mice vs WT mice as the Nlrp3 inflammasome pathway is upstream of IL-IB and important for the transcription and expression of IL-1B. There was no significant difference in TNF-α levels between WGP trained WT and Nlrp3 KO macrophages. Taken together, these results emphasize that WGP-mediated trained immunity is not dependent on the IL-1B/IL-IR pathway.

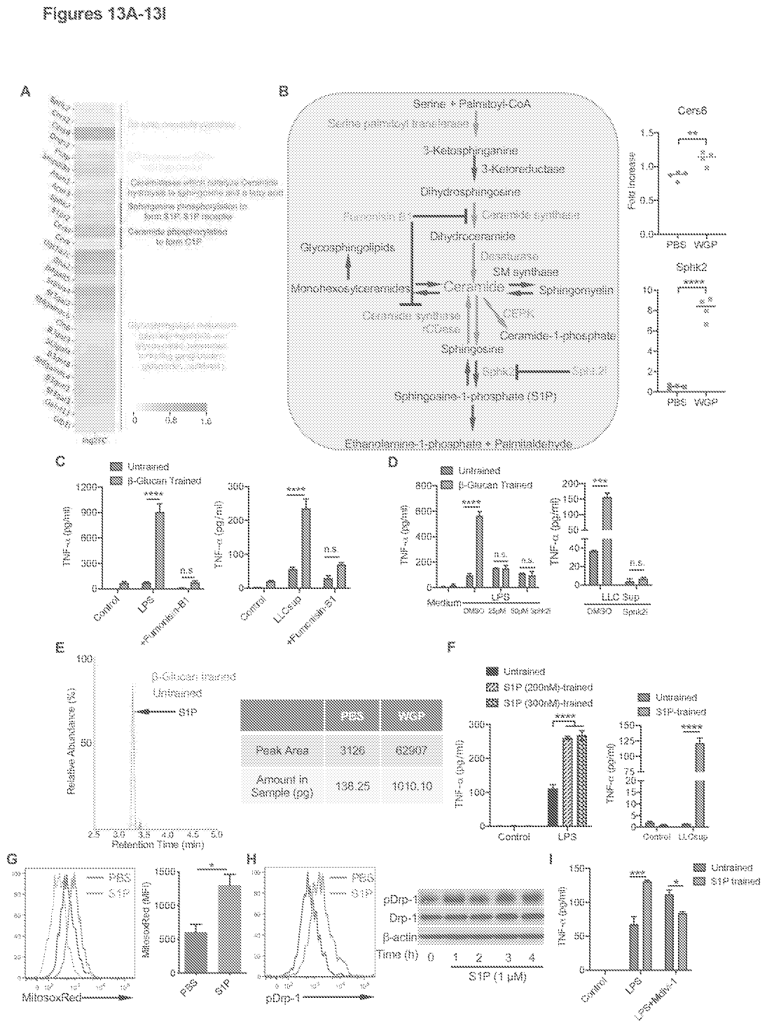

To examine other possible signaling pathways responsible for WGP-mediated trained immunity, we further analyzed our RNAseq data and found that one of the most upregulated genes expressed by WGP-trained IMs is ceramide synthase 6 (CerS6), a gene in the sphingolipid synthesis pathway. Many genes involved in sphingolipid synthesis pathways were also upregulated in WGP-trained IM (FIG. 13A). Genes related to ceramide synthesis (CerS2, CerS6) and metabolism (Sphk2, Asah1, Acer3) were among the highly upregulated genes (FIG. 13A). qRT-PCR analysis also confirmed that CerS6 and sphingosine kinase 2 (Sphk2) mRNA levels were increased in WGP-trained lung IM (FIG. 13B). We next examined whether inhibition of ceramide synthase has any effects on WGP-mediated trained immunity.

Addition of ceramide synthase inhibitor Fumonisin-B1 abrogated WGP-mediated trained response as revealed by TNF-α levels when re-stimulated with both LPS and LLC culture supernatants (FIG. 13C). Ceramide can be synthesized through a de novo synthesis pathway where serine and palmitoyl-CoA undergo a series of reactions to produce ceramide or through a salvage pathway using sphingosine (FIG. 13B). Breakdown of ceramide via ceramidases yields sphingosine which can be phosphorylated via sphingosine kinases (Sphk 1 or Sphk2) to form sphingosine-1-phosphate (S1P). To understand if accumulation of ceramide or generation of S1P is important for WGP-mediated training, we added a specific Sphk2 inhibitor (Sphk2i) in an in vitro training experiment. Sphk2i treatment completely attenuated the trained responses for both LPS and LLC culture supernatant re-stimulation (FIG. 13D), suggesting that production or accumulation of S1P but not ceramide itself is important for the trained response.

To further establish the role of S1P in the WGP-mediated trained response, we measured S1P abundance by Liquid Chromatography-Mass Spectrometry (LC-MS) in PBS and WGP-trained peritoneal macrophages. WGP-trained macrophages showed an increased S1P abundance as compared to PBS controls confirming that WGP-mediated training induces an accumulation of S1P in macrophages (FIG. 13E).

To determine whether S1P accumulation is responsible for the induction of trained immunity in IMs, we performed an in vitro training assay where S1P was used as a training agent. Peritoneal macrophages trained with S1P showed significantly higher TNF-α production compared to the untrained controls upon LPS or LLC culture supernatant restimulation (FIG. 13F). Since we observed that WGP-trained macrophages exhibited an enhanced mtROS production, we next examined whether S1P also induces mtROS production in macrophages. Indeed, S1P-trained peritoneal macrophages exhibited an enhanced mtROS production measured by flow cytometry (FIG. 13G). Previous studies have shown that S1P induces mtROS through the activation/phosphorylation of Dynamin-related protein-1 (Drp-1), translocation of Drp-1 to the mitochondria and subsequent mitochondrial fission. Treatment with S1P also resulted in a significantly increased p-Drp-1 in macrophages (FIG. 13H). Addition of a selective Drp-1 inhibitor Mdivi-1 abrogated S1P-induced trained immunity (FIG. 13I), suggesting that mitochondrial fission may play a critical role in WGP-mediated trained immunity. Collectively, these results suggest a critical role of the sphingolipid pathway, specifically S1P-mediated mtROS production and mitochondrial fission, in WGP-induced trained immunity.

### WGP-Mediated Mitochondrial Fission is Critical for the Trained Response and Metastasis Inhibition

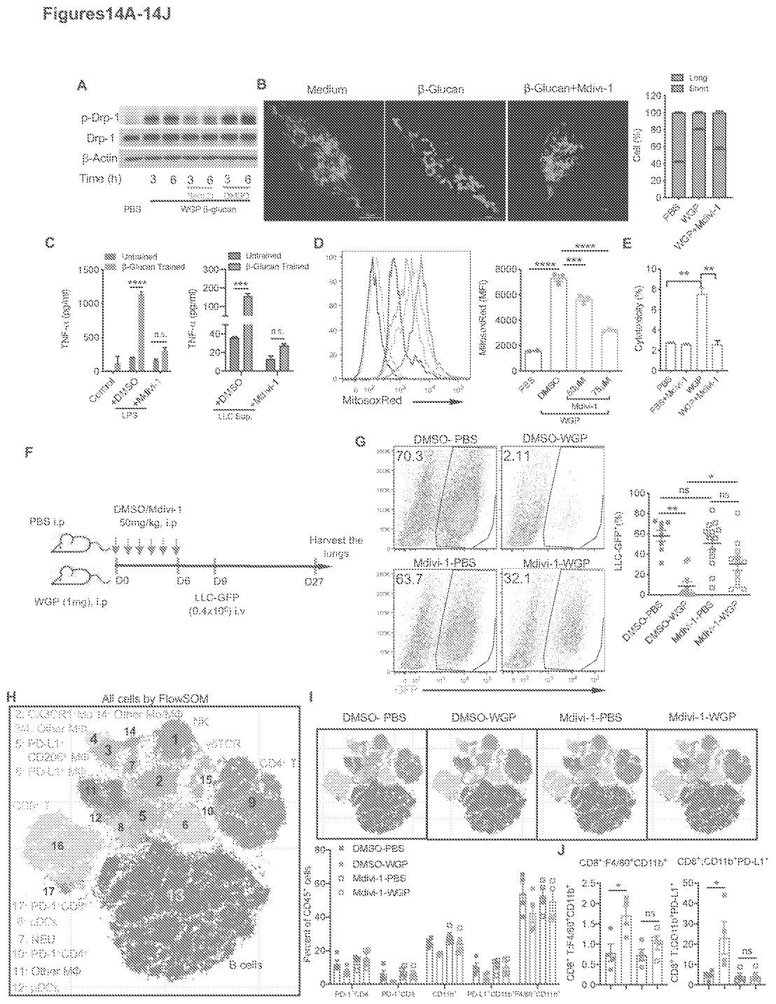

Mitochondrial fission has been reported to induce mtROS and drive a NF-κβ-dependent inflammatory cytokine transcription in macrophages. To further examine the role of mitochondrial fission in WGP-mediated trained immunity, we assessed p-Drp-1 levels in WGP trained peritoneal macrophages in the presence or absence of Sphk2i. WGP trained macrophages showed a significant phosphorylation of Drp-1 (FIG. 14A). Addition of Sphk2i decreased the level of p-Drp-1 but not total Drp-1 (FIG. 14A). WGP-mediated mitochondrial fission was also detected by staining macrophages with a cell membrane permeable dye Tetra-methyl-rhodamine methyl ester (TMRM). Confocal microscopy analysis revealed that WGP training resulted in mitochondrial fragmentation as measured by mitochondrial length. Inhibition of mitochondrial fission by Mdivi-1 abrogated WGP-induced mitochondrial fragmentation (FIG. 14B). To examine whether inhibition of mitochondrial fission also impacts WGP-mediated trained immunity, in vitro training experiments in the presence or absence of Mdivi-1 were performed. Inhibition of mitochondrial fission by Mdivi-1 abrogated WGP-mediated trained responses when re-stimulated with LPS or LLC culture supernatants (FIG. 14C). In addition, Inhibition of mitochondrial fission also abrogated WGP-mediated mtROS production (FIG. 14D) and cytotoxicity against tumor cells (FIG. 7E). These results suggest critical roles of mitochondrial fission in the WGP-induced trained immunity in vitro.

To determine the role of mitochondrial fission in the WGP-mediated trained response in vivo, mice were trained with WGP along with treatment of Mdivi-1 or vehicle control daily throughout the training period (FIG. 14F). The BM and lungs were harvested on day 7 for phenotyping. BM analysis revealed an expansion of both LSKs and MPPs in WGP-trained Mdivi-1- and vehicle control DMSO-treated mice compared to respective untrained controls. There was no significant difference between WGP-trained DMSO-or Mdivi-1-treated mice, suggesting that inhibition of mitochondrial fission does not inhibit WGP-mediated myelopoiesis in the BM.

Similarly, analysis of the lungs also did not show any difference in frequencies of CD11b⁺ myeloid cells and IM in WGP-trained DMSO-vs Mdivi-1 treated. Mdivi-1 treatment therefore did not affect WGP-mediated accumulation of myeloid cells in the lungs. Strikingly, the lung IM trained phenotype in WGP trained mice treated with Mdivi-1 was completely inhibited as revealed by TNF-α levels. In contrast, DMSO-treated control mice showed a significantly enhanced TNF-α expression in lung IMs after WGP training.

Having demonstrated that WGP-induced lung IM trained immunity is dependent on the mitochondrial fission pathway in vivo, we next examined whether inhibition of mitochondrial fission also impacts cancer lung metastasis. To this end, mice were trained with WGP along with treatment of Mdivi-1 or vehicle control daily for 6 days and LLC-GFP cells were injected on day 9. Mice were euthanized on day 27 (FIG. 14F). WGP-trained mice treated with vehicle control showed significantly lower tumor burdens compared to untrained mice (FIG. 14G). However, there was no difference in the tumor burdens between WGP trained mice treated with Mdivi-1 and untrained control mice, suggesting that loss of lung IM training in the presence of Mdivi-1 fails to control tumor metastasis. We also profiled both innate and adaptive T/B cells within the lung by the mass cytometer (FIG. 14H). Although the frequencies of CD4 and CD8 T cells were not changed in WGP-trained mice, the frequencies of PD-1⁺CD4⁺ and PD-1⁺ CD8⁺ T cells were decreased, while Mdivi-1 treatment abolished this effect (FIG. 14I). In contrast, both CD4 and CD8 T cells produced more TNF-α in WGP-trained mice. In addition, the overall F4/80⁺CD11b⁺ lung MAMs and specifically PD-L1⁺/CD206⁺ lung macrophages (clusters 5 and 8) were drastically decreased in WGP-trained mice, and this effect was abolished when mice were treated with Mdivi-1 (FIG. 14I). Consequently, CD8⁺ T cells ratios to these myeloid cells were increased in WGP-trained mice (FIG. 14J). Taken together, these results suggest that mitochondrial fission is essential for both in vitro and in vivo WGP-mediated lung IM trained responses and in vivo cancer metastasis control.

### Discussion

Exploration of trained immunity in the context of tumor therapy is emerging. Trained innate responses exerted by nanobiologics and β-glucan have been reported to induce an anti-tumor effect in primary subcutaneous tumors. However, the mechanisms of how innate immune cells induce a trained response to control cancer metastasis and the etiology of secondary stimuli that elicit trained responses have not been studied. In the tumor microenvironment, immune cells come in contact with not only tumor cells, but also a range of different tumor-derived factors such as cytokines, chemokines, growth factors, DAMPs (ATP, HMGB1, MIF, S100 proteins, hyluronan, heat shock proteins, and calreticulin). In this study, we observed that WGP-trained macrophages induce a trained response in response to stimulation of both tumor cells and tumor-derived factors. We also found that the cytokine MIF is one of the tumor-derived factors that can trigger a trained response when used as a secondary stimulus to stimulate WGP-trained macrophages. This finding suggests that the induction of trained immunity could be part of the immunosurveillance mechanisms and may be able to contain tumor progression and metastasis.

Induction of myelopoiesis by reprogramming HPSCs is a distinct feature of β-glucan-mediated trained response. In line with this phenomenon, we observed the myelopoiesis in the BM upon WGP treatment, leading to a systemic increase in myeloid cells in the spleen, LN, and lungs. In the lung compartment, there are many myeloid cell subsets including IMs and AMs. Upon exposure to intratracheal pneumococcal infections, trained AMs have been reported to protect against recurrent bacterial infections. However, in our study we showed that lung IMs, but not AMs, are trained by WGP treatment. Lung IMs are composed of macrophages from BM while AMs are considered to be tissue resident macrophages. Since WGP training induces BM myelopoiesis, BM-derived macrophages that bear a trained phenotype may traffic into the lung. More important, we showed that WGP-trained lung IMs elicit a vigorous trained response upon challenge with tumor-derived factors including MIF. This data led us to hypothesize that WGP-induced lung IM trained response might be able to control tumor lung metastasis.

We used multiple mouse metastasis models and demonstrated that induction of trained immunity by WGP significantly inhibits tumor metastasis. Mice trained with WGP showed 50-60% tumor-free survival in both LLC and B16F10 tumor models. We also showed the benefit of a systemic effect of WGP training using a liver metastasis model. To further demonstrate the therapeutic efficacy of WGP-mediated trained response in tumor metastasis setting, we used a clinically relevant lung metastasis model and a K-ras^{LA1} GEMM model for spontaneous lung cancer development. Treatment with WGP results in prolonged survival in 4T1 primary breast cancer resected Balb/c mice and significant reduced lung tumor nodules in the spontaneous K-ras^{LA1} lung cancer model.

Surgical resection of primary tumors to minimize the risk of secondary organ metastases is a common practice for patients with early-stage cancer. However, a significant fraction of patients still develops recurrent cancer and metastasis. Previous studies have shown that nearly 30% of women diagnosed with early-stage breast cancer will develop metastatic disease. Therefore, patients who have received surgical excision of primary tumors still require adjuvant therapies to prevent occurrence of metastases. Our data suggest that the induction of trained immunity through modalities such as WGP treatment may provide an option to these patients for preventing potential tumor recurrence and metastasis. In addition, WGP-induced trained immunity could potentially be used in combination with immune checkpoint inhibitors, such as anti-PD-1 therapy, as a new adjuvant regimen for cancer metastasis control. Indeed, anti-PD-1 has been used as adjuvant therapy for high-risk resected stage III melanoma patients and has demonstrated significant prolongations in recurrence-free survival. The protective effect of WGP in these clinically relevant mouse models suggest the possibility that induction of trained immunity, in combination with immune checkpoint blockade, could be an effective adjuvant therapy strategy in surgically resected cancer patients.

In this study, we showed that the WGP-mediated trained immunity functions independently of adaptive T cells and neutrophils. In addition, we showed that lung macrophages are the effector cells that control tumor metastases. A recent study reported that β-glucan-induced granulopoiesis and a neutrophil-mediated anti-tumor response are critical in controlling primary subcutaneous tumors. In our study, however, depletion of neutrophils did not affect WGP-mediated training and metastasis control. The difference may be due to differential tumor models and sources of β-glucan. In addition, depletion of T cells did not affect WGP-mediated training and metastasis control. However, WGP training was found to enhance T cell responses in tumor-bearing mice. RNAseq analysis showed an enrichment of immune effector functions and antigen processing and presentation in WGP-trained IMs. Thus, enhanced T cell effector function could be a result of a WGP-mediated increase in macrophage antigen processing and presentation.

Induction of trained immunity has been attributed to the epigenetic and metabolic reprogramming through a HIF-1α/mTOR signaling pathway and an inflammasome-dependent IL-1β pathway. However, we showed that inhibition of the HIF-1α, mTOR, Nlrp3 or IL-1β pathway did not affect WGP-mediated metastasis control. Metabolic reprogramming of innate immune cells is one of the hallmarks of trained immunity, and previous studies have reported increased glycolysis, glutamine, or cholesterol biosynthesis pathway as important metabolic changes required for inducing trained immunity.

In this study, we established a novel sphingolipid-mediated mitochondrial fission pathway that is responsible for WGP-induced trained immunity and subsequent metastasis control. WGP-trained lung IMs have upregulated expression of genes related to sphingolipid metabolism, especially the accumulation of S1P which results in the activation of Drp-1 and therefore mitochondrial fission. Increased mitochondrial fission further leads to an increased mtROS production, which results in enhanced macrophage cytotoxicity against tumor cells. Metabolites such as mevalonate and lipoproteins have been reported to induce a trained innate phenotype. Our data demonstrate a previously unidentified metabolic pathway, as we observed that S1P treatment in macrophages induces a trained phenotype. In addition, we showed that S1P induces enhanced mtROS production. S1P mediates macrophage differentiation, migration and survival and therefore is an important determinant of macrophage function.

In addition, S1P induces ROS production, promotes TNF-α production, regulates histone acetylation by inhibiting HDAC1, and induces mitochondrial fission. Our data demonstrate that S1P is also a critical metabolite in the induction of trained immunity in macrophages by WGP. Based on these findings, we propose a new pathway for WGP-mediated trained immunity, where WGP treatment leads to an enhanced sphingolipid synthesis and subsequent accumulation of S1P in macrophages. Increases of S1P result in Drp-1 activation and subsequent mitochondrial fission, leading to an enhanced mtROS production and cytotoxicity against tumor cells. These trained macrophages exhibit significant antitumor immunity where they were shown to inhibit tumor progression and metastasis. Our findings emphasize the potential of using WGP to induce trained immunity, and highlight that the induction of trained immunity can be used as an effective approach to control cancer metastasis.

### Materials and Methods

Mice. WT C57B1/6 and Balb/c mice were purchased from the Jackson Laboratory. HIF-1α^{f/f}/LysM-cre mice on a C57B1/6 background were generated by crossing HIF-α flox/flox mice with Lysozyme-cre (LysM-cre) mice. Similarly, Raptor^{f/f}/LysM-cre mice and Rictor^{f/f}/LysM-cre were also generated by crossing Raptor flox/flox or Rictor flox/flox mice with LysM-cre mice. IL-IR KO mice were purchased from the Jackson Laboratory. Global MIF KO mice were graciously provided by Dr. Robert Mitchell from University of Louisville. K-ras^{LA1} and Nlrp3 KO mice were kindly provided by Dr. Haribabu Bodduluri from University of Louisville. OT-II mice were bred and maintained at the Rodent Rearing Facility (RRF) facility of University of Louisville. Mice were housed in a specific pathogen-free facility at University of Louisville. All the mice were at least 6 weeks of age and all the experiments were carried out in accordance with all relevant laws and institutional guidelines provided by the RRF and approved by the Institutional Animal Care and Use Committee (IACUC) of the University of Louisville.

Preparation of β-glucan. Saccharomyces cerevisiae derived particulate β-glucan, WGP (Biothera), was dissolved in PBS and sonicated at 40 pulses twice for 10 seconds each on ice using a Qsonica Q55-110 Q55 Sonicator (Cole-Parmer) before treatment or injection.

PFIR characterization of WGP. A 20 μL aqueous solution containing 20 μg/mL WGP was dropped on silicon wafer and dried in air. The topography, IR absorption, stiffness and adhesion images of WGP particles were acquired using a home-built PFIR microscopy that was described in detail previously. Briefly, PFIR spectral scans between 900-1800 cm⁻¹ were first performed at different locations on the WGP surface. Then PFIR signal at 1040 cm⁻¹, which is the characteristic signal of polysaccharides, was acquired over the entire WGP surface. During the IR scanning, stiffness and adhesion properties were simultaneously acquired over the WGP.

Fluorescence imaging of WGP phagocytosis by GFP-Dectin-1 expressing RAW264.7 macrophages. Cells were plated a 35 mm glass coverslip in complete DMEM media overnight. WGP particles were added to cells and fluorescence images were acquired using a Re-scan confocal microscope equipped with a 1.64 N.A.×100 TIRF objective and a Hamamatsu CMOS camera. Three-dimensional fluorescence image stacks were projected to two-dimensional images based on maximum intensity using ImageJ (NIH).

Preparation of DTAF-labeled WGP. 5-([4,6-Dichlorotriazin-2-yl]amino) fluorescein hydrochloride (DTAF, 2 mg/ml, Sigma Aldrich) was mixed with 20 mg/ml WGP in borate buffer (pH 10.8) and incubated at room temperature for 8 h with continuous shaking. The mixture was then centrifuged, washed with cold sterile endotoxin-free DPBS (Sigma Aldrich) for 5 times until the supernatant had no visible traces of DTAF. The concentration was then adjusted to 10 mg/ml in endotoxin-free DPBS and maintained at 4° C. for storage. Mice were intraperitoneally (i.p.) injected with 1 mg of DTAF-WGP and euthanized after 72 hrs to harvest the bone marrow and the lungs.

Isolation and culture of peritoneal macrophages. Mice were euthanized with CO₂ inhalation and injected i.p. with 5 ml of sterile cold, complete DMEM medium (Sigma Aldrich) followed by gentle massage of the peritoneum. The peritoneal fluid was then collected, centrifuged at 1600 rpm for 5 min. The cell pellet was washed, counted, and plated at appropriate numbers in complete DMEM. The cells were incubated for 2-4 hours at 37° C. and 5% CO₂ to allow peritoneal macrophages attached to the plates. The floating cells were aspirated and plates were gently washed with sterile pre-warmed medium and then added with fresh complete DMEM. For comparison studies between different mouse strains, peritoneal cells were stained with viability dye and anti-F4/80 antibodies (Biolegend) and F4/80⁺ macrophages were sorted by FACS Aria III (BD Bioscience).

In vitro training and re-stimulation of peritoneal macrophages. Peritoneal macrophages were treated with 25 μg/ml of WGP or polystyrene beads (3 μm, Sigma-Aldrich) and incubated for 24 h at 37°° C. with 5% CO₂. Macrophages were then washed with pre-warmed complete DMEM at least twice to remove excess WGP or polystyrene beads, added with fresh complete DMEM and incubated 6 days. On day 7, macrophages were re-stimulated with different stimuli such as LPS (10 ng/ml) (Sigma), tumor cells, tumor cell culture supernatants (40%) or rMIF (100 ng/ml) (kindly provided by Dr. Robert Mitchell) for 24 h. The culture supernatants were harvested and assayed for TNF-α by ELISA. For cell culture supernatants, tumor cell lines including LLC, B16F10, and EL4, and control cell line MLE-12 were used.

These cell lines were cultured (1 million/4 ml complete DMEM) in a 6-well plate and incubated for 72 h. The supernatants were harvested and stored at −80° C. in aliquots.

ELISA for TNF-α and MIF. TNF-α in the culture supernatants was measured using TNF-α ELISA kit (Biolegend) and MIF levels in the culture supernatants were quantified using mouse MIF ELISAkit (R&D Systems) per the manufacturer's instruction.

In vivo WGP training. Mice were injected IP with one dose of WGP (1 mg/200 μl PBS) on day 0 and euthanized on day 7 to assess the lung, BM, spleen and lymph node phenotype. Mice treated with 1 mg polystyrene beads were used as controls.

Preparation of lung, BM, spleen, and lymph node single cell suspensions. Mice were euthanized and lungs were harvested and cut into smaller pieces using sterile scissors and transferred into 15 ml centrifuge tubes containing 4.5 ml of complete DMEM and 0.5 ml of 10X digestion buffer (mixture of collagenase, hyaluronidase and deoxyribonucleosidase) (Sigma Aldrich). The tubes were then incubated in a rotating incubator set at 37° C. and 5% CO₂ for 30 min. After incubation, the digestion was immediately stopped by addition of 5 ml of complete DMEM medium. The suspension was then filtered through a sterile 40 μm cell strainer (VWR) into petri dishes and extra tissue chunks were further mashed with syringe columns. The single cell suspensions were centrifuged and cell pellets were added ACK lysis buffer (ammonium chloride 8.29 g/L, potassium bicarbonate 1 g/L, disodium ethylenediaminetetraacetate 0.0372 g/L; membrane filtered and maintained at a pH of 7.4±0.2) for about 1 min followed by complete DMEM wash twice. Cells were then suspended in complete DMEM medium. BM suspensions were prepared by harvesting mouse tibia bones and flushing them with DMEM medium. The cells were harvested, centrifuged and allowed for RBC lysis using 1 ml ACK lysis buffer. The suspensions were washed twice and suspended in complete DMEM. Spleen and Lymph node single cell suspensions were prepared by gently mashing the tissues in the filter buckets and pelleted by centrifugation. RBC lysis was performed for the spleen cells followed by washing. The cells were then suspended in complete DMEM.

Surface staining and flow cytometry. Lung single cell suspensions were washed with PBS and Fc blocker was added and incubated for 10 min at 4° C. For lung macrophages, cells were stained with viability dye eFluor 780 (eBioscience), anti-CD45-PerCP-Cy5.5, anti-CD11b-APC and anti-F4/80-PE antibodies (Biolegend). For monocyte subsets, cells were stained with viability dye eFluor 780, anti-CD11b-APC, anti-Ly6C-PerCP-Cy5.5, anti-Ly6G-PE and anti-CX3CR1-FITC (Biolegend). For T cells, cells were stained with viability dye eFluor 780, anti-CD45-PerCP-Cy5.5, anti-CD4-APC and anti-CD8-FITC antibodies (Biolegend). Cells were incubated at 4° C. for 30 min, washed with cold PBS, filtered, and collected using FACACanto flow cytometer (BD Bioscience). All flow data were analyzed using FlowJo software (BD).

BM phenotyping. BM cells were stained for surface markers anti-CD19-APC, anti-Ter119-APC, anti-CD11b-APC, anti-Ly6C/G-APC, anti-CD3-APC as Lineage markers along with anti-Ly6A/E-APC-Cy7 (Sca-1), anti-CD117-PE-Cy7 (c-kit), anti-CD48-FITC and anti-CD150-PE-Cy5 (SLAM) (Biolegend) for Lin⁻Sca-1⁺c-kit⁺ LSK populations and Lin⁻Sca-1⁺c-kit⁺ CD48⁺CD150⁺ multipotent progenitors (MPPs).

Isolation of lung alveolar macrophages and interstitial macrophages. Single cell lung suspensions were washed with 1 ml of sterile cold PBS and added Fc blocker for 10 min at 4° C. followed by staining with anti-CD45-PerCP-Cy5.5, anti-CD11b-APC, anti-F4/80 -PE and viability dye eFluor 780. The cells were then washed with 2 ml of sterile autoMACS running buffer, filtered and resuspended in appropriate volume of running buffer for acquisition by FACS Aria III sorter. The alveolar macrophages were gated at viable CD45⁺CD11b⁻F4/80⁺ population and the interstitial macrophages were gated at viable CD45⁺CD11b⁺F4/80⁺ population and collected on individual tubes containing an appropriate volume of a mixture of 50% FBS (Atlanta Biologicals),40% PBS (Sigma) and 10% HEPES (Corning).

Tumor metastasis model. Mice were treated with intraperitoneal injections of WGP (1 mg in 200 μl PBS/mouse) or sterile PBS (200 μl PBS) on day 0 and intravenously administered with tumor cells (LLC-GFP, B16.F10 or EL4) in 200 μl of sterile PBS on day 7. The mice were then allowed for tumor development for at least 14-16 days and euthanized to assess the tumor development in the lungs or maintained for the assessment of long-term survival. For the short-term tumor protocols, mice were injected with 1×10⁶ LLC-GFP cells on day 7 after WGP treatment and allowed for tumor seeding for 24-48 hrs after which the mice were euthanized and assessed for tumor burden in the lungs. For LLC-GFP model, tumor-bearing mice were euthanized after 14-16 days of tumor injection and the lungs were harvested, digested, and processed to obtain a single cell suspension. Cells were then stained with viability dye eFluor 780 and anti-CD45-PerCp-Cy5.5antibodies. The frequency of LLC-GFP cells was determined by flow cytometry. Cells were gated on viable, CD45 negative population.

Ex vivo stimulation of lung, spleen, and lymph node macrophages. Mononuclear cell suspensions from lung, spleen, and lymph node were plated in a 24-well plate (2 million/well) and stimulated with LPS (10 ng/ml) or LLC culture supernatant (40%) or rMIF (100 ng/ml) and incubated at 37° C. with 5% CO₂ for 5-6 hours in the presence of 1X brefeldin A (Biolegend). The cells were then harvested using a mini-cell scrapper (United Biosystems), pelleted and stained for surface markers followed by intracellular TNF-α staining.

Ex vivo stimulation of lung T cells. Lung single cell suspensions were plated in a 24 well plate, stimulated with phorbol 12-myristate 13-acetate (PMA, 50 ng/ml) and ionomycin (500 ng/ml) (Sigma, EMD Millipore Corp) in the presence of brefeldin A (Biolegend) and incubated at 37° C. with 5% CO₂ for 4 hours. The cells were then harvested and stained for the intracellular expression of TNF-α and IFN-γ on CD4⁺ and CD8⁺ T cells.

Lung histopathology. Lung tissues were stored in 10% formalin for 24 h at room temperature. The lungs were then rinsed with PBS and transferred to 70% ethanol for paraffin embedding using standard procedures. Lungs were sectioned (5 μm) and stained for Hematoxylin and Eosin (H &E). Images of the representative sections were then scanned using an Aperio Scanscope.

Intracellular cytokine staining. Mononuclear cells were first stained for surface markers and washed with cold PBS. The supernatants were then dumped, 500 μl of fixation buffer (Biolegend) was added. The tubes were briefly vortexed and incubated at room temperature for 20 min. After incubation, the fixation was stopped by the addition of 1 ml of 1X permeabilization buffer (Biolegend) and centrifuged at 1600 rpm for 5 minutes at 4° C. followed by one more wash using 1X permeabilization buffer. Cells were then stained with anti-TNF-α-PE or anti-IFN-γ-PE (Biolegend) along with respective isotype controls and incubated at 4° C. for at least 1 h or overnight. The sample tubes after incubation were washed with 1 ml of 1 X permeabilization buffer, filtered and followed by suspension in 250 μl of 1X permeabilization buffer for acquisition by flowcytometer.

Intracellular staining for FoxP3. Mononuclear cells were surface stained and washed with cold PBS, and fixed with 1 ml of fixation buffer. The tubes were briefly vortexed and incubated at 4° C. for 30 min followed by washing with 2 ml of 1X permeabilization buffer twice. Anti-FoxP3-PE (Biolegend) antibodies along with isotype control antibodies were added to the tubes and incubated at 4° C. for at least 1 h or overnight. The tubes were further washed with 1 ml of 1X permeabilization buffer, filtered and suspended in 250 μl of 1X permeabilization buffer for acquisition by flow cytometer.

CFSE labeling of OT-II T cells. Spleens from OT-II mice were harvested and splenocytes were resuspended in 1 ml of pre-warmed 0.1% bovine serum albumin (BSA) to which 2 μM of CFSE (CellTrace™ CFSE Cell Proliferation Kit, Invitrogen) was added and incubated at 37° C. with 5% CO₂ for 10 min with frequent mixing during the incubation. After incubation, 5 ml of ice-cold complete RPMI1640 (Sigma) medium was added to the tubes to quench the labeling and maintained on ice for 5 min. The cells were washed twice with RPMI1640 and then suspended inappropriate volumes of fresh medium.

T cell proliferation assay. Metastasis-associated macrophages (MAMs) (viable CD45⁺CD11b⁺F4/80⁺) from the lungs of tumor-bearing PBS vs WGP-trained mice were sorted using FACS Aria III sorter as mentioned earlier. The MAMs were then co-cultured with CFSE-labelled OT-II splenocytes at different ratios in the presence of ovalbumin (OVA) (200 μg/ml) (Sigma) and incubated at 37° C. with 5% CO₂ for 96 h. Cells were harvested and analyzed for the T cell proliferation and also stained for intracellular expression of IFN-γ on CD4⁺ T cells using flow cytometry. For T cell proliferation, the cells were stained for surface marker with viability dye eFluor 780 and anti-CD4-APC antibodies (Biolegend). For the intracellular expression of IFN-γ on CD4⁺ T cells, the cells were stimulated with PMA/Ionomycin in the presence of Brefeldin A for 4 h and performed intracellular staining. Cells were acquired by flow cytometer.

In vivo depletion of macrophages, T cells, and neutrophils. Depletion of macrophages were performed by intravenous (i.v) injection of Clodrosome (Encapsula NanoSciences) 200 μl/mouse at days −2, 2 and 5. To deplete CD4 and/or CD8 T cells, mice were i.p. injected with two doses of 200 μg of anti-CD4 or anti-CD8 mAb or both at day −1 and day 4 during the training period. Depletion of neutrophils was performed by i.p. injection of 300 μg of anti-Ly6G mAb (Bio X cell) at day −1, 2 and 6 during the training period. Mice were trained with either PBS or WGP (1 mg/200 μl) at day 0. Mice were then challenged with 0.4×10⁶ LLC-GFP tumor cells i.v. on day 7 and euthanized after 14-16 days of tumor challenge to analyze tumor burden in the lungs.

Triple negative breast cancer 4T1 model. 6 weeks old female Balb/c mice were subcutaneously implanted with 4T1 tumor cells (1×10⁶/mouse) in the fourth mammary pad. When tumor sizes reached to 3-4 mm in diameter, tumors were surgically resected. Two days after surgery, mice were injected i.p. with WGP (1 mg) or PBS and observed for long-term survival.

Spontaneous K-ras^{LA1} lung cancer model. 6 weeks old K-ras^{LA1} mice were i.p. injected with WGP (1 mg) or PBS. Treatments were repeated every three weeks at 9 weeks, 12 weeks, and 15 weeks of age. The mice were then euthanized at 17 weeks to harvest the lungs. The tumor nodules in the lungs were counted and the lungs were then sectioned and stained for H & E for histopathological analysis.

RNA extraction for RNA sequencing. Lung IMs from PBS and WGP trained mice were sorted using FACS Aria III cell sorter. Cells were washed twice with ice-cold PBS and stored in TRIzol (AmbionR). RNA was extracted using a QIAGEN RNAeasy Kit (QIAGEN) and checked for integrity using the Agilent Bioanalyzer 2100 system (Agilent Technologies). Quantification of the extracted RNA was performed using a Qubit fluorometric assay (Thermo Fischer Scientific). Poly-A enriched mRNA-seq libraries were prepared according to the

Universal Plus mRNA-Seq kit standard protocol (Tecan Genomics) using a total of 10 ng RNA. All the samples were then ligated with Illumina adapters and barcoded individually. RNA extraction kit as per the manufacturer's instruction and sent to the Genomics core at the University of Louisville for cDNA library preparation and sequencing. Absence of adapter dimers and consistent library size of approx. 300 bp was confirmed using the Agilent Bioanalyzer 2100. The library concentration and sequencing behavior was assessed in relation to a standardized spike-in of PhIX using a Nano MiSeq sequencing flow cell from Illumina. 1.8 pM of the pooled libraries with 1% Phi.X spike-in was loaded on one NextSeq 500/550 75 cycle High Output Kit v2 sequencing flow cell and sequenced on the Illumina NextSeq 500 sequencer targeting 60 M 1×75 bp reads per sample.

RNA Sequencing. Sequencing libraries were prepared using the Universal Plus mRNA-seq kit with NuQuant® library quantification (NuGen). Quality control (QC) of the raw sequence data was performed using FastQC (version 0.10.1) (Andrews, 2015a) with good quality indicated for all samples. The sequences were aligned to the mouse reference genome (assembly mm 10.fa) using STAR (version 2.6) (Dobin et al., 2013). Raw gene counts were obtained using HTSeq (version 0.10.0) (Anders et al., 2015) and normalized using the Relative Log Expression (RLE) method, followed by filtering to exclude genes with fewer than 10 counts across the samples. Differential expression was performed using DESeq2. Functional annotation analysis of the differentially expressed genes was performed using Gene Set Enrichment Analysis (GSEA).

Phagocytosis assay. Lung cell suspensions were washed with HEPES dilutes in antibiotic-free complete RPMI1640 and resuspended in 100 μl of the same solution in non-adherent culture tubes. The reconstituted particles as indicated in the pHrodo™ Green S. aureus Bioparticles™ Phagocytosis Kit for Flow Cytometry (Thermo Fisher Scientific) was added to the lung single cell suspensions and incubated at 37° C. for 1 h with gentle mixing every 15 min. The incubation was then stopped by addition of 1 ml cold PBS. Cells were centrifuged and followed by surface staining for lung macrophages using viability dye eFluor 780, anti-CD45-PerCP-Cy5.5, anti-CD11b-PE-Cy7 and anti-F4/80-APC. Cells were acquired by flow cytometer.

Cytotoxicity assay. Lung IMs from the PBS vs WGP-trained mice were sorted and co-cultured with LLC cells at different ratios in a 96-well plate. The plates were then incubated at 37° C. with 5% CO₂ for 12-16 h. Upon completion of incubation, the supernatants were harvested and assayed for the release of lactate dehydrogenase as per the instructions described in the CyQUANT™ LDH Cytotoxicity Assay Kit (Invitrogen). For the cytotoxicity assay in the presence of Mdivi-1 (10 μM) (Sigma Aldrich), peritoneal macrophages were in vitro trained with PBS or WGP in the presence of Mdivi-1 or DMSO for 6 days. The macrophages were then harvested, counted, and co-cultured with LLC cells at a ratio of 10:1 followed by the lactate dehydrogenase assay for cytotoxicity. In another set of experiments, IMs from the lungs of PBS vs WGP-trained mice were harvested, sorted, and co-cultured with LLC cells (10:1) in the presence of N-acetyl-L-Cysteine (NAC, 1 mM) (Sigma Aldrich) or DMSO and assayed for cytotoxicity.

qRT-PCR. PBS vs WGP-trained lung IMs were sorted using a FACS Aria III sorter, washed twice with ice-cold PBS and frozen in TRIzol (Ambion®) solution for storage at −80° C. The frozen TRIzol samples were thawed, and RNA was extracted using a standard phenol-chloroform method. Total RNA was quantified using a NanoDrop Spectrophotometer and used for cDNA preparation using an iScript™ DNA Synthesis Kit (BIO-RAD) in a BioRad MyiQ single color RT-PCR detection system. qRT-PCR was performed using an iQ™ SYBR® Green Supermix (BIO-RAD) in a CFX Connect Real-time System.

Mitochondrial ROS quantitation. Peritoneal macrophages were treated with WGP or S1P in the presence or absence of Mdivi-1 and incubated at 37° C. with 5% CO₂ for 24 h. The cells were then harvested, washed with PBS and pelleted by centrifugation at 1600 rpm for 5 min at room temperature. The pellets were then stained with MitoSOX Red dye (5 μM) (Invitrogen) at 37° C. for 15 min. The cells were washed with pre-warmed HBSS followed by another wash with PBS at room temperature. The cells were then stained with viability dye and anti-F4/80 and then acquired by flow cytometry. Flow cytometry for p-Drp-1. Peritoneal macrophages were treated with WGP (25 μg/ml) or S1P (1 μM) and incubated at 37° C. with 5% CO₂ for 3-4 h. Following the completion of incubation, the cells were quickly washed with cold PBS to stop the activation. The cells were harvested and washed with PBS. The cells were then fixed with 4% formaldehyde for 15 min at room temperature, washed with PBS and permeabilized using ice-cold 100% methanol on ice for a minimum of 10 min. The cells were then stained with primary antibody (p-Drp1) (Cell Signaling) for 1 hour at room temperature followed by secondary antibody (Anti-rabbit IgG) (Biolegend) for 30 min at room temperature as instructed in the Cell Signaling phospho-stain protocol and analyzed using flow cytometry.

Western blot analysis for total Drp-1 and p-Drp-1. Treated or untreated peritoneal macrophages were washed with sterile PBS and incubated with lysis buffer on ice for 30 min. The cell lysates were then harvested and centrifuged at 14000 rpm and 4° C. for 15 min. The supernatants were then collected into separate 1.5 ml tubes and assayed for protein concentration using Pierce BCA Protein Assay Kit (ThermoFisher Scientific). The samples were further denatured and ran for SDS-polyacrylamide gel electrophoresis (SDS-PAGE) followed by transfer to PVDF membrane. The PVDF membrane was blocked with 5% BSA in Tris buffered saline-Tween (TBST) solution for 1 h at room temperature with shaking, washed with TBST three times for 10 minutes each and stained for primary anti-Drp-1 (Cell signaling Technology), p-Drp-1 (Cell signaling Technology) and β-actin (Sigma) individually by incubating overnight at 4° C. The membrane was then washed for 3 times using 1X TBST for 10 min each and stained for secondary antibodies, incubated for 1 h at room temperature followed by washing and addition of detection reagent (ECL plus Western Blotting Detection System; Amersham Biosciences).

S1P quantification by Liquid Chromatography-Mass Spectrometry (LS-MS/MS). S1P was extracted using a well-established method with minor modifications (Bligh and Dyer, 1959). PBS vs WGP trained peritoneal macrophages (1×10⁶) suspended in 400 μl of PBS were treated with 1.5 ml of MeOH/CHCl₃ at a concentration of 2:1 v/v ratio and vortexed for 1 min. Induction of phase separation was then allowed by the addition of 500 μl of CHCl₃ to the mixture followed by the addition of 500 μl of H₂O. The bottom layer was collected and allowed for drying under gentle N₂ flow. 50 μl of MeOH/CHCl₃ (1:2, v/v) was added to the tubes for final analysis by LC-MS/MS (Waters ACQUITY UPLC Systems with 2D Technology coupled with Waters Xevo TQ-Smicro triple quadrupole Mass Spectrometer). Further S1P analysis was performed using Multiple reaction monitoring (MRM) in positive ionization mode.

In vitro training with S1P. Peritoneal macrophages were stimulated with 200 nM or 300 nM of S1P for 24 h at 37° C. and 5% CO₂. The macrophages were then washed and allowed for resting. On day 7, the macrophages were stimulated with LPS (10 ng/ml) or LLC culture supernatant (40%) for 24 h and the supernatants were collected for the quantification of TNF-α using ELISA. For another set of in vitro-training experiment, macrophages were trained with S1P in the presence of Mdivi-1 (10 μM) during S1P training and throughout the resting period. Cells were restimulated with LPS and the supernatants were collected to determine TNF-α levels by ELISA.

Treatment of peritoneal macrophages with inhibitors in vitro: Plated peritoneal macrophage cultures were treated with Mdivi-1 (10 μM), sphingosine kinase-2 inhibitor (ABC294640; 50 μM), Fumonisin-B1 (50 μM), and corresponding vehicle controls along with treatment with WGP and incubated for 24 h followed by washing with complete DMEM. The inhibitors were further added to the macrophage cultures and incubated at 37° C. and 5% CO₂ for 6 more days. On day 7, the macrophages were washed once with pre-warmed complete DMEM and added LPS (10 ng/ml) or LLC culture supernatant (40%) for re-stimulation.

Confocal microscopy. Poly-L-Lysine-treated glass coverslips were added to 12 well plates to which appropriate numbers of macrophages were added along with 2 ml of sterile complete DMEM. Cells were trained with WGP alone or in combination with Mdivi-1 (10 μM) for 7 days as previously described. After 7 days of incubation, the media was removed, and the cells were washed with sterile PBS followed by addition of TMRM dye (Tetramethylrhodamine, methyl ester, Sigma) for 20 min. After incubation, the cells were washed with PBS and fixed with 1 ml of 4% formalin at room temperature for 20-30 min. The cells were washed thrice with PBS and the glass slides were removed carefully with the help of forceps and mounted on the slides using mounting reagents and left overnight to dry at room temperature or stored at 4° C. protecting from the light. The slides were then read using a Nikon Confocal microscope. Mitochondrial fragment lengths were measured by counting at least 100 mitochondria per sample using an Image-J software.

In vivo Mdivi-1 treatment. 6 weeks old C57B1/6 female mice were i.p. injected with Mdivi-1 (50 mg/kg) or DMSO along with suitable carriers (5% Tween-80 and 40% PEG300) starting day 0 for 6 days. Mdivi-1/DMSO treated mice were also treated with PBS or WGP at day 0. Mice were euthanized at day 7 to harvest the BM and lungs for phenotyping using flow cytometry. In tumor challenging experiment, Mdivi-1/DMSO-treated PBS vs WGP trained mice were i.v. injected with 0.4×10⁶ LLC-GFP cells on day 9. Mice were euthanized on day 27. Lungs were then analyzed for tumor burden using flow cytometry.

CyTOF mass cytometry staining, data acquisition, and analysis. Lung single cell suspensions were prepared as described earlier and ex vivo stimulated with PMA/Ionomycin for 4 h at 37° C., 5% CO₂. The cells were then harvested, washed with PBS and transferred to sterile-capped culture tubes. Cells were then stained for viability using 5 μM cisplatin (Fluidigm) in serum-free RPMI 1640 for 5 min at RT and washed with complete RPMI 1640. Cells were stained with the surface markers antibodies for 30 min at RT and washed twice with Maxpar cell staining buffer (Fluidigm) followed by fixation with 1 ml of 1X Maxpar Fix I buffer for 30 min at RT. Upon fixation, cells were washed twice with 2 ml of 1X Maxpar Perm-S buffer for 5 min at 800 g, stained for the cytoplasmic/secreted antibodies and incubated for 30 min at RT. Following incubation, cells were washed twice with 1 ml of 1X Maxpar Perm-S buffer for 5 min at 800 g. The cells were then suspended in 1 ml of 1X Maxpar nuclear antigen staining buffer (Fluidigm) and incubated for 30 min at RT, washed twice with Maxpar nuclear antigen staining permeability buffer (Fluidigm) at 800 g for 5 min each. Cells were stained for nuclear antigens and incubated at RT for 30 min, washed twice with Maxpar nuclear antigen staining permeability buffer and fixed with 1.6% formaldehyde for 10 min at RT. The fixed cells were then centrifuged to remove the formaldehyde at 800 g for 5 min and incubated with 125 nM of intercalator iridium (Fluidigm) overnight at 4° C. After overnight incubation, the cells were washed twice with cell staining buffer, pelleted and kept on ice until acquisition using CyTOF. Prior to acquisition, cells were suspended in a 1:9 solution of Cell acquisition solution: EQ 4 element calibration beads (Fluidigm) and acquired using a Helios CyTOF system. Upon acquisition of the samples, the .FCS files were normalized to .fcs files using the CyTOF software for analysis. The data acquired by CyTOF were then analyzed using FlowJo10 and the FlowSOM softwares.

### BMDM Training and Adoptive Transfer Protocol

BMDMs were trained in the presence of Mdivi-1 or DMSO. BMDMs (1×10⁶) were then adoptively transferred intravenously to WT mice twice 2 days apart. Two days later recipient mice were challenged with LLC-GFP tumor cells. Mice were euthanized at day 21 post tumor challenge to analyze the tumor burden and T cell phenotype in the lungs.

Statistical Analysis. Results are represented as mean±SEM. Data were analyzed using a two-tailed Student's t test or Mann-Whitney U-test. Multiple-group comparisons were performed using a one-way or two-way ANOVA followed by Tukey's multiple comparisons test. Statistical significance was set at p<0.05. All statistical analyses were performed using GraphPad Prism Software Version 8 (GraphPad Inc., La Jolla, CA).

### EXAMPLE 3

### Adoptive Transfer of Trained Innate Immunity Significantly Reduces Tumor Metastasis

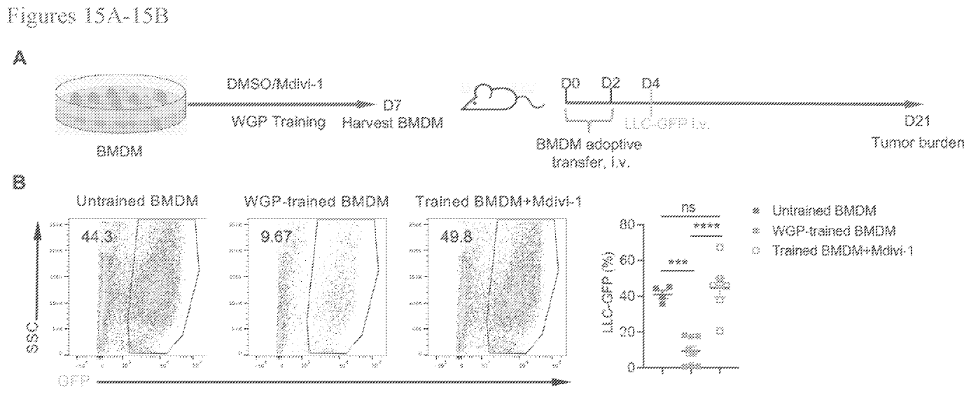

To examine whether ex vivo beta-glucan trained macrophages could be used for adoptive cell therapy, bone marrow-derived macrophages (BMDM) were used for a proof-of-concept study. To this end, BMDM were trained with WGP beta-glucan for 7 days and then adoptively transferred into naïve mice. Recipient mice were then challenged with LLC-GFP tagged lung cancer cells. Mice were euthanized at day 21 (FIG. 15A). As shown in FIG. 15B, mice received WGP-trained BMDM had significantly reduced lung metastasis compared to mice received untrained BMDM. In addition, inhibition of mitochondrial fission using small molecule inhibitor Mdivi-1 completely abrogated WGP-trained BDMD-mediated effect. These data show that trained innate immune cells are useful as a novel adoptive cell therapy in cancer.

### EXAMPLE 4

### WGP-Trained Human Monocytes Significantly Reduce Lung Cancer Development

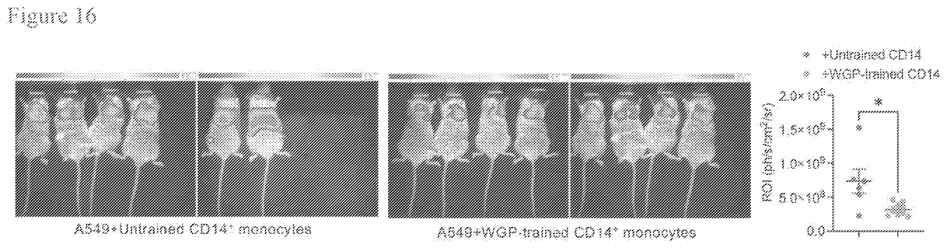

To further confirm human relevance, human monocytes were trained ex vivo with WGP beta-glucan and then performed admix experiment with luciferase-tagged human non-small cell lung cancer cell line A549. Lung tumor progression was monitored by in vivo imaging analysis. As shown in FIG. 16, mice received WGP-trained monocytes admixed with A549 had significantly reduced tumor burden compared to mice received untrained monocytes. Taken together, these data show that trained innate immune cells by beta-glucan WGP are useful as a novel approach for cancer treatment.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a hyperproliferative disorder comprising administering a therapeutically effective amount of a therapeutic agent comprising yeast-derived particulate β-glucan.

2. The method of claim 1, wherein the hyperproliferative disorder is a cancer.

3. The method of claim 2, wherein the cancer is pancreatic cancer.

4. A method inducing CCR2-dependent influx of immune cells to a pancreas comprising administering a therapeutically effective amount of a therapeutic agent comprising a yeast-derived particulate β-glucan.

5. The method of claim 4, wherein the immune cells are monocytes or macrophages.

6. (canceled)

7. A method of recruiting anti-tumor, innate immune cells to pancreatic ductal adenocarcinoma (PDAC) tumor microenvironment (TME) comprising administering a therapeutically effective amount of a therapeutic agent comprising a yeast-derived particulate β-glucan.

8. (canceled)

9. The method of claim 3, wherein the cancer is ductal adenocarcinoma (PDAC).

10. The method of claim 2, wherein the cancer is lung cancer.

11. (canceled)

12. The method of claim 1, wherein the yeast-derived particulate β-glucan comprises whole β-Glucan particles (WGP).

13. The method of claim 1, further comprising administering an anti-PD-L1 immunotherapy.

14. The method of claim 13, wherein the anti-PD-L1 immunotherapy is an anti-Programmed Death ligand-1 (anti-PD-L1) immunotherapy.

15. The method of claim 14, wherein the anti-PD-L1 immunotherapy is a monoclonal antibody therapy.

16. The method of claim 1, further comprising administering anti-Programmed Death-1 (PD-1) or anti-CTLA-4 immunotherapy.

17. The method of claim 16, wherein the anti-PD-1 or anti-CTLA-4 immunotherapy is an anti-PD-1 or anti-CTLA-4 mAb therapy.

18. The method of claim 1, wherein the yeast-derived particulate β-glucan is derived from Saccharomyces cerevisiae.

19. The method of claim 1, wherein the yeast-derived particulate β-glucan is in the form of whole β-glucan particles (WGP) derived from Saccharomyces cerevisiae.

20. The method of claim 19, wherein the WGP comprise 2-4 micron hollow yeast cells made of highly concentrated (1,3) β-glucans.

21. The method of claim 1, wherein the yeast-derived particulate β-glucan is administered by means of injection.

22. The method of claim 21, wherein the injection is an intraperitoneal injection.

23. The method of claim 21, wherein the injection is an intra-tumoral injection.

24. The method of claim 1, wherein the yeast-derived particulate β-glucan is administered orally.

25-30. (canceled)
